(19) United States
(12) Patent Application Publication
(10) Pub. No.: US 2026/0327260 A1
(43) Pub. Date: Oct. 22, 2026

(54) PHOSPHONOALANINE OLIGOPEPTIDES AND METHODS OF MAKING AND USE THEREOF

(71) Applicant: Ohio State Innovation Foundation, Columbus, OH (US)

(72) Inventors: Kou-San JU, Dublin, OH (US); Yeying ZHANG, South San Francisco, CA (US); Jerry CUI, Charlotte, NC (US)

(21) Appl. No.: 19/474,459

(22) PCT Filed: Apr. 12, 2024
(86) PCT No.: PCT/US2024/024268

§ 371 Date: Oct. 10, 2025

Related U.S. Application Data
(60) Provisional application No. 63/459,009, filed on Apr. 13, 2023.

(51) Int. Cl. C07K 5/08; A01N 63/50; A01P 1/00; A01P 13/00; C12P 21/02

(52) U.S. Cl. CPC C07K 5/0827; A01N 63/50; A01P 1/00; A01P 13/00; C12P 21/02

(57) ABSTRACT

Disclosed herein are compositions and methods of making and use thereof. For example, disclosed herein are compounds defined by Formula I. Also disclosed herein are compounds comprising a head group and a tail group, the head group being bound to the tail group using one or more enzymes derived from Streptomyces, wherein the head group comprises a phosphonic acid, a phosphinic acid, or a derivative thereof, and the tail group comprises one or more (canonical or non-canonical) amino acids. In some examples, the compound is a Streptomyces isolate or a derivative or salt thereof. Also disclosed herein are compositions comprising any of the compounds disclosed herein. Also disclosed herein are methods of making and use of any of the compounds or compositions disclosed herein.

### CROSS-REFERENCE TO RELATED APPLICATIONS

This application claims the benefit of priority to U.S. Provisional Application No. 63/459,009, filed Apr. 13, 2023, which is hereby incorporated herein by reference in its entirety.

### STATEMENT OF GOVERNMENT SUPPORT

This invention was made with government support under grant/contract no. GM137135 awarded by the National Institutes of Health. The government has certain rights in the invention.

### REFERENCE TO SEQUENCE LISTING

The sequence listing submitted on Apr. 12, 2024, as an .XML file entitled “103361-488WO1_ST26” created on Apr. 12, 2024, and having a file size of 28,601 bytes is hereby incorporated by reference pursuant to 37 C.F.R. § 1.52(e)(5).

### BACKGROUND

Some phosphonate natural products, including isolates from naturally-occurring microorganisms, have been shown to have inhibitory activities. The inhibitory activities underly their development as antibiotics and pesticides. Most bio-active phosphonate natural products have been isolated from Actinobacteria. Many plant, animal, and insect pathologies have poor or no modalities of control and new compositions are needed. The compositions and methods discussed herein address these and other needs.

### SUMMARY

In accordance with the purposes of the disclosed compositions and methods as embodied and broadly described herein, the disclosed subject matter relates to compositions and methods of making and use thereof.

For example, disclosed herein are compounds defined by Formula I:

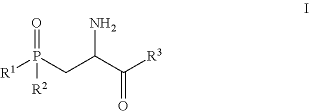

wherein R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a}; R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b}; R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids); R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and R^{x} and R^{y} are independently selected from hydrogen, or substituted or unsubstituted C₁-C₅ alkyl, or substituted or unsubstituted C₁-C₅ acyl; or a derivative or salt thereof; with the proviso that the compound is not phosphonoalamide A, B, C, D, E, or F.

Also disclosed herein are compounds comprising a head group and a tail group, the head group being bound to the tail group using one or more enzymes derived from Streptomyces, wherein the head group comprises a phosphonic acid, a phosphinic acid, or a derivative thereof, and the tail group comprises one or more (canonical or non-canonical) amino acids, with the proviso that the compound is not phosphonoalamide A, B, C, D, E, or F.

In some examples, the compound is a Streptomyces isolate or a derivative or salt thereof.

Also disclosed herein are compositions comprising any of the compounds disclosed herein.

Also disclosed herein are nucleic acids encoding any of the compounds or compositions disclosed herein, vectors encoding said nucleic acids, cells comprising said vectors, and cells comprising any of the compounds or compositions disclosed herein.

Also disclosed herein are methods of making any of the compounds or compositions disclosed herein.

Also disclosed herein are methods of making a compound comprising a head group and a tail group, the head group being bound to the tail group, wherein the head group comprises a phosphonic acid, a phosphinic acid, or a derivative thereof, and the tail group comprises one or more (canonical or non-canonical) amino acids, using one or more enzymes derived from Streptomyces, with the proviso that the compound is not phosphonoalamide A, B, C, D, E, or F.

Also disclosed herein are methods of use of any of the compounds, compositions, nucleic acids, vectors, or cells disclosed herein.

Also disclosed herein are methods of reducing the activity of bacteria, the methods comprising exposing the bacteria to an effective amount of any of the compounds, compositions, nucleic acids, vectors, or cells disclosed herein.

Also disclosed herein are methods of reducing bacterial population, the methods comprising exposing the bacteria to an effective amount of any of the compounds, compositions, nucleic acids, vectors, or cells disclosed herein.

Also disclosed herein are methods of killing bacteria, the methods comprising exposing the bacteria to an effective amount of any of the compounds, compositions, nucleic acids, vectors, or cells disclosed herein.

Also disclosed herein are methods for treating, preventing, inhibiting, and/or ameliorating a disease or disorder in a plant or a subject in need thereof, the methods comprising administering to the plant or subject a therapeutically effective amount of any of the compounds, compositions, nucleic acids, vectors, or cells disclosed herein.

Also disclosed herein are methods for treating, preventing, inhibiting, and/or ameliorating a microbial infection in a plant or a subject, comprising administering to the plant or subject an effective amount of any of the compounds, compositions, nucleic acids, vectors, or cells disclosed herein.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A. The genome neighborhood of the phosphonoalamide biosynthetic gene cluster. Cloned cosmid inserts are indicated by brackets.

FIG. 1B. ³¹P NMR analysis of heterologous expression strain extracts.

FIG. 2A. Biosynthetic scheme for L-PnAla formation.

FIG. 2B. ³¹P NMR analysis of PnaAD reactions demonstrating conversion of PEP to PnAla.

FIG. 2C. ³¹P NMR analysis of PnaA reverse reactions demonstrating conversion of PnAla to PnPy.

FIG. 3A. Potential biosynthetic routes to the phosphonoalamide A.

FIG. 3B. LC-HRMS analyses of PnaB reactions. PnaB ligates PnAla to Ala-Val (iii) but only in the presence of ATP (iv). PnAla-Ala (i) and Ala-Val (ii) are not formed by PnaB.

FIG. 3C. LC-IRMS analyses of PnaC reactions. PnaC ligates Ala to Val (ii) but only when provided ATP (iv). PnAla-Ala (i) and PnAla-Ala-Val (iii) are not formed by PnaC.

FIG. 3D. ³¹P NMR analysis of PnaB ligation reactions of PnAla and chemically synthesized Ala-Val (i), Val-Val (ii), and Ala-Ile (iii). The reaction containing PnaC, PnAla, and Ala-Val served as a negative control (iv).

FIG. 4A-FIG. 4B. (A) Summary of dipeptide products from synthesis reactions using PnaC. (B) Summary of tripeptide products from one-pot synthesis reactions using PnaC and PnaB. Shaded cells indicate product formation and the degree of ion intensity for each respective product m/z.

FIG. 5. The pepM gene neighborhoods of strains containing the phosphonoalamide biosynthetic gene cluster.

FIG. 6. Synteny analysis between the phosphonoalamide gene neighborhood of S-515 and the genomes of strains lacking pepM.

FIG. 7. ¹H-³¹P NMR spectra of S. lividans 66 attB::pKSJ588 extract.

FIG. 8. LC-HRMS analysis of phosphonates produced by heterologous expression strains.

FIG. 9. SDS-PAGE of purified Pna proteins. 10 μg of VlpA, PnaB, and PnaC and 3 ug of PnaA were loaded, with asterisks indicating bands of expected size.

FIG. 10. ¹H-³¹P HMBC of the PnaD-PnaA coupled reaction.

FIG. 11. ³¹P NMR spectra of PnaD-PnaA reactions with different amino donors.

FIG. 12. ¹H-³¹P HMBC spectra of PnaA catalyzed transamination of OAA to Asp and PnAla to PnPy.

FIG. 13A-FIG. 13C. LC-HRMS analysis of PnaA catalyzed transamination reactions. A) EIC of PnAla from PnaA-PnaD reactions. B) Reaction scheme of PnPy derivatization with phenylhydrazine. C) EIC of phenylhydrazine-derivatized PnPy formed from PnaA reactions.

FIG. 14A. LC-HRMS analysis of derivatized PnaA catalyzed transamination reactions. EIC of phenylhydrazine-derivatized PnPy (257.0332 m/z) from PnaA reactions.

FIG. 14B. LC-HRMS analysis of derivatized PnaA catalyzed transamination reactions. MS/MS of the 257.0332 m/z ion with a retention time of 9.2 min.

FIG. 14C. LC-HRMS analysis of derivatized PnaA catalyzed transamination reactions. Mass spectrum at 12.72 min.

FIG. 14D. LC-HRMS analysis of derivatized PnaA catalyzed transamination reactions. MS/MS of the 515.0738 m/z ion with a retention time of 12.72 min.

FIG. 15. Timecourse of PnPy formation in the PnaA-catalyzed transamination of PnAla to PnPy using OAA as the keto-acid acceptor.

FIG. 16. Timecourse of PnAla formation in the PnaA-catalyzed transamination of Asp to OAA using PnPy as the keto-acid acceptor.

FIG. 17A-FIG. 17B. Transamination reactions with varied amounts of Asp or OAA after 2 hours. A) The PnaA forward reaction containing 1.5-10 mM Asp. B) The PnaA reverse reaction containing 1.5-10 mM OAA.

FIG. 18A. Kinetic analyses for the conversion of PnPy and L-Asp to L-PnAla and OAA by PnaA. Apparent steady-state parameters were determined by coupling the formation of OAA to its reduction to malate (MAL) and the concurrent oxidation of NADH by malate dehydrogenase (MDH).

FIG. 18B. Kinetic analyses for the conversion of PnPy and L-Asp to L-PnAla and OAA by PnaA. The Michaelis-Menten plot fit to the resulting measurements.

FIG. 19A. Kinetic analyses for the conversion of L-PnAla and OAA to PnPy and L-Asp by PnaA. Apparent steady-state parameters were determined by coupling the formation of PnPy to its reduction to PnLac and the concurrent oxidation of NADH by PnPy reductase (VlpB).

FIG. 19B. Kinetic analyses for the conversion of L-PnAla and OAA to PnPy and L-Asp by PnaA. The Michaelis-Menten plot fit to the resulting measurements.

FIG. 20A. Kinetic analyses for the PEP conversion to L-PnAla in a coupled reaction with PnaD and PnaA. Apparent steady-state parameters were determined by coupling the formation of OAA to its reduction and the concurrent oxidation of NADH by malate dehydrogenase (MDH).

FIG. 20B. Kinetic analyses for the PEP conversion to L-PnAla in a coupled reaction with PnaD and PnaA. The Michaelis-Menten plot fit to the resulting measurements.

FIG. 21A. Kinetic analyses of 2-KG conversion to 2-hydroxyglutarate by 3-phosphoglycerate dehydrogenase SerA. Apparent steady-state parameters were determined from the oxidation of NADH by SerA.

FIG. 21B. Kinetic analyses of 2-KG conversion to 2-hydroxyglutarate by 3-phosphoglycerate dehydrogenase SerA. The Michaelis-Menten plot fit to the resulting measurements.

FIG. 22A. Kinetic analyses for the conversion of OAA to malate by 3-phosphoglycerate dehydrogenase SerA. Apparent steady-state parameters were determined from the oxidation of NADH by SerA.

FIG. 22B. Kinetic analyses for the conversion of OAA to malate by 3-phosphoglycerate dehydrogenase SerA. The Michaelis-Menten plot fit to the resulting measurements.

FIG. 23A. Kinetic analyses for the conversion of L-Asp and OAA to 2-KG and L-Asp by PnaA. Apparent steady-state parameters were determined by coupling the formation of 2-KG to its reduction to 2-hydroxyglutarate and the concurrent oxidation of NADH by SerA.

FIG. 23B. Kinetic analyses for the conversion of L-Asp and OAA to 2-KG and L-Asp by PnaA. The Michaelis-Menten plot fit to the resulting measurements.

FIG. 24A. Kinetic analyses for the conversion of 2-KG and L-Asp to L-Glu and OAA to by PnaA. Apparent steady-state parameters were determined by coupling the formation of OAA to its reduction to malate and the concurrent oxidation of NADH by malate dehydrogenase.

FIG. 24B. Kinetic analyses for the conversion of 2-KG and L-Asp to L-Glu and OAA to by PnaA. The Michaelis-Menten plot fit to the resulting measurements.

FIG. 25A. LC-MS analyses of PnaB and PnaC reactions for dipeptide ligation. Reaction scheme for the ligation of PnAla and Ala-Val.

FIG. 25B. LC-MS analyses of PnaB and PnaC reactions for dipeptide ligation. LC-MS analysis of the reaction containing PnaB, ATP, L-PnAla, and filtered PnaC reaction (PnaC, L-Ala, L-Val, ATP).

FIG. 25C. LC-MS analyses of PnaB and PnaC reactions for dipeptide ligation. LC-MS analysis of the reaction containing PnaC, ATP, L-PnAla, and filtered PnaC reaction (PnaC, L-Ala, L-Val, ATP).

FIG. 26A-FIG. 26D. LC-MS analyses of PnaB ligation reactions with chemically synthesized dipeptides. Using Ala-Val (A), Thr-Val (B), Ala-Ile (C), and Val-Val (D).

FIG. 27. LC-HRMS detection of PnAla and phosphonoalamides within culture extracts of S. lividans 66 attB::pKSJ595. As Ile and Leu are isomers, it was not possible to distinguish whether a specific tripeptide ion contained Ile or Leu from LC-HRMS alone.

FIG. 28. Full summary of PnaC dipeptide products. All dipeptides produced by PnaC, organized by N- and C-terminal residues. Products shown in black were directly observed by LC-HRMS and verified by MS/MS. Products shown in white were inferred from the observation of an N-terminal L-PnAla tripeptide containing the stated dipeptide.

FIG. 29. Full summary of PnaBC tripeptide products. All tripeptides produced by the combined reaction of PnaB and PnaC, organized by central and C-terminal residues. All products were observed by LC-HRMS and verified by MS/MS. (DA=Phosphonoalanine.

FIG. 30. All N-terminal L-PnAla-containing tripeptides produced by PnaBC. A representation of all tripeptides produced by PnaBC. The sequence of each tripeptide is read from the inner circle outwards, such that PnAla-Ala-Val is represented by following the PnAla circle, the Ala inner ring, and the Val outer wedge. Amino acids are colored by their general properties.

FIG. 31A-FIG. 31C. Impact of ion suppression on EIC spectra. A representative example of ion suppression leading to artifacts in EIC peaks. A) The EIC for Ala-Glu in the PnaC reaction containing Ala+Glu. B) The TIC for the same reaction. C) The overlay of the TIC over the Ala-Glu EIC demonstrates how ion suppression has caused a break in the EIC peak.

FIG. 32A-FIG. 32D. Background noise in EIC spectra. A representative example of background ions leading to noise in EIC spectra. A) The EIC for Asn-Val in the PnaC reaction containing Asn+Val. B) The same EIC in a PnaC reaction completely lacking Asn and Val, containing only Gly. C) The mass spectra for the window from 3.7-3.8 min in the Asn+Val reaction demonstrates a m/z 232.1282, −4.31 ppm from the theoretical m z for protonated Asn-Val. D) The same region in the Gly reaction shows no such peak.

FIG. 33A-FIG. 33E. Contaminating parent ions in the selection window. The LC-HRMS used in this study has a minimum 0.4 m/z window for ion selection. A) The full MS/MS spectra for the Thr-Val species, m/z 219.1339. B) Looking closer into the selection window, four parent ions can be seen. C) Only one of the four parent ions is within 5 ppm of the theoretical m z for protonated Thr-Val. D) The fragment ions which can be attributed to Thr-Val show similar EIC peaks. E) The other fragment ions have EICs which match the contaminating ions.

### DETAILED DESCRIPTION

The compositions, methods, and systems described herein may be understood more readily by reference to the following detailed description of specific aspects of the disclosed subject matter and the Examples included therein.

Before the present compositions, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, Me refers to a methyl group; OMe refers to a methoxy group; and i-Pr refers to an isopropyl group.

As used herein, agriculturally acceptable salts and esters refer to salts and esters that exhibit herbicidal activity, or that are or can be converted in plants, water, or soil to the referenced herbicide. Exemplary agriculturally acceptable esters are those that are or can be hydrolyzed, oxidized, metabolized, or otherwise converted, e.g., in plants, water, or soil, to the corresponding carboxylic acid which, depending on the pH, may be in the dissociated or undissociated form.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), birds, and insects. “Subject” can also include a mammal, such as a primate or a human. Thus, the subject can be a human or veterinary patient. The term “patient” refers to a subject under the treatment of a clinician, e.g., physician.

As used herein, antimicrobials include, for example, antibacterials, antifungals, and antivirals. As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “reduce” or other forms of the word, such as “reducing” or “reduction,” refers to lowering of an event or characteristic (e.g., microbe population/infection). It is understood that the reduction is typically in relation to some standard or expected value, in other words it is relative, but that it is not always necessary for the standard or relative value to be referred to. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of microbial infections, “treating,” “treat,” and “treatment” as used herein, refers to partially or completely inhibiting or reducing the microbial infections which the subject is suffering. In one embodiment, this term refers to an action that occurs while a patient is suffering from, or is diagnosed with, the microbial infections, which reduces the severity of the condition, or retards or slows the progression of the condition. Treatment need not result in a complete cure of the condition; partial inhibition or reduction of the microbial infections is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “molecular weight” refers to number average molecular weight as measured by ¹H NMR spectroscopy, unless indicated otherwise.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

As used herein, the term “encapsulation,” or grammatical equivalent, refers to the process of confining an individual nucleic acid molecule within a nanoparticle.

As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used inter-changeably.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. In some examples, the term “nucleic acid” as used herein means natural and synthetic DNA, RNA, oligonucleotides, oligonucleosides, and derivatives thereof. For ease of discussion, such nucleic acids are at times collectively referred to herein as “constructs,” “plasmids,” or “vectors.”

The term “gene” as used in this specification refers to a segment of deoxyribonucleotides (DNA) possessing the information required for synthesis of a functional biological product such as a protein or ribonucleic acid (RNA).

The term “genetic engineering” is used to indicate various methods involved in gene manipulation including isolation, joining, introducing of gene(s) as well as methods to isolate select organisms containing the manipulated gene(s).

As specified herein, the term “DNA construct” refers to a sequence of deoxyribonucleotides including deoxyribonucleotides obtained from one or more sources.

The term “gene expression” refers to efficient transcription and translation of genetic information contained in concerned genes.

The term “recombinant” cells or population of cells refers to cells or population of cells into which an exogenous nucleic acid sequence is introduced using a delivery vehicle such as a plasmid.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some examples, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The term “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

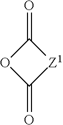

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

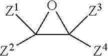

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein comprises the formula —S—.

The term “thiol” as used herein is represented by the formula —SH.

“R₁,” “R₂,” “R₃,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amino group, an alkyl group, a halide, and the like. Depending upon the groups that are selected, a first group can be incorporated within a second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds comprising phosphonic/phosphinic acids or derivatives thereof.

For example, disclosed herein are compounds defined by Formula I:

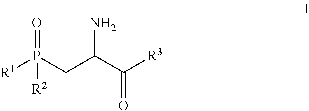

wherein
R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b};
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

In some examples of Formula I, R¹ is OR^{a} and/or R² is OR^{b}. In some examples of Formula I, R¹ is OR^{a} and R² is OR^{b}. In some examples of Formula I, R¹ is OR^{a} and/or R² is OR^{b}, wherein R^{a} and/or R^{b} is hydrogen. In some examples of Formula I, R¹ is OR^{a}, R² is OR^{b}, R^{a} is hydrogen, and R^{b} is hydrogen.

In some examples, of Formula I, R³ is one or more amino acids (e.g., one or more canonical or non-canonical amino acids). In some examples of Formula I, R³ is one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

In some examples of Formula I, R¹ is OR^{a}, R² is OR^{b}, R³ is one or more amino acids (e.g., one or more canonical or non-canonical amino acids), or a combination thereof. In some examples of Formula I, R¹ is OR^{a}, R² is OR^{b}, and R³ is one or more amino acids (e.g., one or more canonical or non-canonical amino acids). In some examples of Formula I, R¹ is OR^{a}, R² is OR^{b}, R^{a} is H, R^{b} is H, and R³ is one or more amino acids (e.g., one or more canonical or non-canonical amino acids).

In some examples, the compound is defined by Formula II.

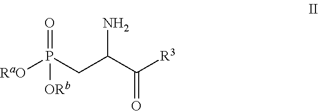

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

In some examples of Formula II, R^{a} and/or R^{b} is hydrogen. In some examples of Formula II, R^{a} and R^{b} are hydrogen.

In some examples, of Formula II, R³ is one or more amino acids (e.g., one or more canonical or non-canonical amino acids). In some examples of Formula II, R³ is one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

In some examples of Formula II, R^{a} is hydrogen, R^{b} is hydrogen, R³ is one or more amino acids (e.g., one or more canonical or non-canonical amino acids), or a combination thereof. In some examples of Formula I, R^{a} is H, R^{b} is H, and R³ is one or more amino acids (e.g., one or more canonical or non-canonical amino acids).

In some examples, the compound is defined by Formula III:

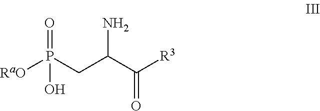

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

In some examples of Formula III, R^{a} is hydrogen.

In some examples, of Formula III, R³ is one or more amino acids (e.g., one or more canonical or non-canonical amino acids). In some examples of Formula III, R³ is one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

In some examples of Formula III, R^{a} is hydrogen and R³ is one or more amino acids (e.g., one or more canonical or non-canonical amino acids).

In some examples, the compound is defined by Formula IV:

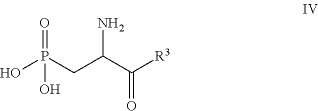

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids); and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

In some examples of Formula IV, R³ is one or more amino acids (e.g., one or more canonical or non-canonical amino acids). In some examples of Formula IV, R³ is one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

In some examples, the compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof. In some examples, the compound comprises PnAla-Ala-Val, PnAla-Val-Val, PnAla-Ala-Ile, PnAla-Thr-Val, a derivative or salt thereof, or a combination thereof. In some examples, the compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof.

In some examples, the compound comprises PnAla-Ala-Ala, PnAla-Ala-Val, PnAla-Ala-Ile, PnAla-Ala-Leu, PnAla-Ala-Met, PnAla-Ala-Phe, PnAla-Ala-Tyr, PnAla-Ala-Trp, PnAla-Ala-Ser, PnAla-Ala-Thr, PnAla-Ala-aThr, PnAla-Ala-Asn, PnAla-Ala-Gln, PnAla-Ala-Arg, PnAla-Ala-His, PnAla-Ala-Lys, PnAla-Ala-Glu, PnAla-Ala-Gly, PnAla-Ala-Pro, PnAla-Val-Val, PnAla-Val-Ile, PnAla-Val-Leu, PnAla-Val-Met, PnAla-Val-Phe, PnAla-Val-Tyr, PnAla-Val-Trp, PnAla-Val-aThr, PnAla-Val-Gln, PnAla-Val-Pro, PnAla-Ile-Ile, PnAla-Ile-Phe, PnAla-Ile-aThr, PnAla-Leu-Leu, PnAla-Met-Val, PnAla-Met-Leu, PnAla-Met-Met, PnAla-Met-Trp, PnAla-Phe-Val, PnAla-Phe-Ile, PnAla-Phe-Leu, PnAla-Phe-Met, PnAla-Phe-Phe, PnAla-Phe-Tyr, PnAla-Phe-Trp, PnAla-Phe-Thr, PnAla-Phe-aThr, PnAla-Tyr-Val, PnAla-Tyr-Leu, PnAla-Tyr-Met, PnAla-Tyr-Phe, PnAla-Tyr-Tyr, PnAla-Tyr-Trp, PnAla-Tyr-Gly, PnAla-Trp-Val, PnAla-Trp-Ile, PnAla-Trp-Leu, PnAla-Trp-Met, PnAla-Trp-Phe, PnAla-Trp-Tyr, PnAla-Trp-Trp, PnAla-Trp-Thr, PnAla-Trp-Lys, PnAla-Trp-Gly, PnAla-Ser-Val, PnAla-Ser-Ile, PnAla-Ser-Leu, PnAla-Ser-Met, PnAla-Ser-Phe, PnAla-Ser-Tyr, PnAla-Ser-Ser, PnAla-Ser-Thr, PnAla-Ser-aThr, PnAla-Thr-Val, PnAla-Thr-Ile, PnAla-Thr-Leu, PnAla-Thr-Phe, PnAla-Thr-Trp, PnAla-aThr-Val, PnAla-aThr-Ile, PnAla-aThr-Leu, PnAla-aThr-Met, PnAla-aThr-Phe, PnAla-aThr-aThr, PnAla-Gln-Val, PnAla-Gln-Ile, PnAla-Gln-Leu, PnAla-His-Phe, PnAla-His-Trp, PnAla-Gly-Val, PnAla-Gly-Ile, PnAla-Gly-Leu, PnAla-Gly-Met, PnAla-Gly-Phe, PnAla-Gly-Tyr, PnAla-Gly-Trp, PnAla-Gly-aThr, PnAla-Gly-Gly, a derivative or salt thereof, or a combination thereof.

In some examples, the compound is a salt. In some examples, the compound is a salt form of Formula I, Formula II, Formula III, Formula IV, or a combination thereof with a counterion. In some examples, the compound is a salt form of Formula II with a counterion. In some examples, the compound is a salt form of Formula II with a counterion and the salt form of the compound is selected from the group consisting of:

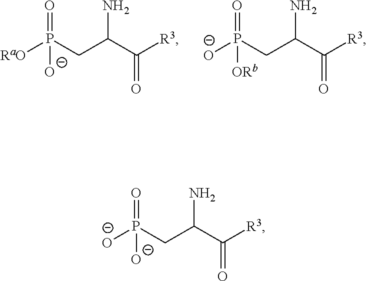

and combinations thereof. In some examples, the compound is a salt form of PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, and/or PnAla-Thr-Val, with a counterion.

In some examples, the counterion is a monovalent or divalent counterion. In some examples, the counterion is selected from the group consisting of sodium, potassium, calcium, lithium, magnesium, manganese, ammonium, iron, and combinations thereof.

In some examples, the compound is a potassium salt, sodium salt, calcium salt, iron salt, ammonium salt, or a combination thereof. In some examples, the compound comprises an agriculturally acceptable salt thereof and/or a pharmaceutically acceptable salt thereof.

Also disclosed herein are compounds comprising a head group and a tail group, the head group being bound to the tail group using one or more enzymes derived from Streptomyces, wherein the head group comprises a phosphonic acid, a phosphinic acid, or a derivative thereof, and the tail group comprises one or more (canonical or non-canonical) amino acids, with the proviso that the compound is not phosphonoalamide A, B, C, D, E, or F. In some examples, the head group comprises phosphonoalanine (PnAla) or a derivative thereof. In some examples, the tail group comprises one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val). In some examples, the compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof. In some examples, the compound comprises PnAla-Ala-Val, PnAla-Val-Val, PnAla-Ala-Ile, PnAla-Thr-Val, or a combination thereof. In some examples, the compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof. In some examples, the head group is N-terminal. In some examples, the compound is a salt. In some examples, the compound is a potassium salt, sodium salt, calcium salt, iron salt, ammonium salt, or a combination thereof. In some examples, the compound comprises an agriculturally acceptable salt thereof and/or a pharmaceutically acceptable salt thereof. In some examples, the compound is a compound of Formula I.

In some examples, the compound is a di-peptide or a tri-peptide.

In some examples, the compound is a Streptomyces isolate or a derivative or salt thereof.

### Compositions

Also disclosed herein are compositions comprising any of the compounds disclosed herein. In some examples, the compositions further comprise one or more agriculturally acceptable and/or pharmaceutically acceptable carriers. In some examples, the composition comprises a pharmaceutical composition, an agricultural composition, or a combination thereof.

In some examples, the composition comprises a pesticide. In some examples, the composition comprises an herbicide.

In some examples, the composition exhibits antimicrobial activity. In some examples, the composition results in at least 5 log reduction in a population of microbes.

In some examples, the composition further comprises a solvent, a carrier, an excipient, or a combination thereof. In some examples, the composition further comprises an agriculturally acceptable adjuvant or carrier.

In some examples, the composition is formulated for delivery to a plant or animal.

In some examples, the composition is formulated for delivery to a plant. In some examples, the plant is a crop.

In some examples, the composition is formulated for delivery to an animal. In some examples, the animal is a companion animal, livestock, research animal, insect, or human.

Also disclosed herein are nucleic acids encoding any of the compounds or compositions disclosed herein. Also disclosed herein are vectors encoding said nucleic acids. Also disclosed herein are cells comprising said vectors. Also disclosed herein are cells comprising any of the compounds or compositions disclosed herein. In some examples, the cell comprises a Streptomyces cell.

### Methods of Making

Also disclosed herein are methods of making any of the compounds disclosed herein.

For example, the methods can comprise a biosynthetic method. In some examples, the method can use one or more enzymes derived from Streptomyces.

For example, also disclosed herein are methods of making a compound comprising a head group and a tail group, the head group being bound to the tail group, wherein the head group comprises a phosphonic acid, a phosphinic acid, or a derivative thereof, and the tail group comprises one or more (canonical or non-canonical) amino acids, using one or more enzymes derived from Streptomyces, with the proviso that the compound is not phosphonoalamide A, B, C, D, E, or F.

In some examples, the head group comprises PnAla or a derivative thereof. In some examples, the tail group comprises one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

In some examples, the compound is any of the compounds disclosed herein.

In some examples, the one or more enzymes comprise one or more ATP-grasp enzymes.

In some examples, the one or more enzymes are encoded by a gene comprising at least 90% identity to pnaB, pnaC, or a combination thereof.

In some examples, the method proceeds via a convergent pathway.

In some examples, the method comprises contacting a first amino acid and a second amino acid with a first enzyme, to thereby form a first compound comprising the first amino acid bound to the second amino acid. In some examples, the first enzyme is encoded by a gene comprising at least 90% identity to pnaC. In some examples, the first enzyme comprises PnaC. In some examples, the first amino acid and/or the second amino acid each independently comprises Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), Valine (Val), a derivative thereof, or a combination thereof.

In some examples, the first compound comprises Ala-Gly, Ala-Ala, Ala-Val, Ala-Ser, Ala-Thr, Ala-Cys, Ala-Leu, Ala-Ile, Ala-Met, Ala-Asn, Ala-Gln, Ala-Lys, Ala-Arg, Ala-Phe, Ala-Trp, Ala-Tyr, Ala-His, Gly-Gly, Gly-Val, Gly-Leu, Gly-Ile, Ser-Gly, Ser-Val, Ser-Ser, Ser-Leu, Ser-Ile, Ser-Met, Val-Val, Met-Met, Ala-L-allo-Thr, Ser-L-allo-Thr, or a combination thereof.

In some examples, the method further comprises a third amino acid or the first compound, and a carboxylate with a second enzyme, the first compound being a nucleophile, the carboxylate comprising a phosphonic acid, a phosphinic acid, or a derivative thereof, to thereby form a second compound comprising the third amino acid or the first compound bound to the carboxylate. In some examples, the second enzyme is encoded by a gene comprising at least 90% identity to pnaB. In some examples, the second enzyme comprises PnaB. In some examples, the carboxylate comprises phosphonoalanine (e.g., PnAla).

In some examples, the second compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof. In some examples, the second compound comprises PnAla-Ala-Val, PnAla-Val-Val, PnAla-Ala-Ile, PnAla-Thr-Val, or a combination thereof. In some examples, the second compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof.

In some examples, the method is further performed in the presence of one or more additional components. In some examples, the method is further performed in the presence of ATP.

### Methods of Use

Also disclosed herein are methods of use of any of the compounds, compositions, nucleic acids, vectors, or cells disclosed herein.

For example, also disclosed herein are methods of using any of the compounds, compositions, nucleic acids, vectors, or cells as an antimicrobial, an herbicide, a pesticide, or combination thereof, for example to control (e.g., treat, reduce, inhibit, and/or ameliorate) an undesirable population.

In some examples, the methods comprise using any of the compounds, compositions, nucleic acids, vectors, or cells as a pesticide.

In some examples, the methods comprise using any of the compounds, compositions, nucleic acids, vectors, or cells to control (e.g., treat, reduce, inhibit, and/or ameliorate) an undesirable population in plants. In some examples, the method comprises contacting the plants or the locus thereof with or applying to the soil or water any of the compounds, compositions, nucleic acids, vectors, or cells. In some examples, the methods further comprise applying an additional pesticide.

In some examples, the undesirable population is an herbicide resistant or tolerant population, a pesticide resistant or tolerant population, an antimicrobial resistant or tolerant population, or a combination thereof. In some examples, the undesirable population comprises bacteria.

Also disclosed herein are methods of reducing the activity of bacteria, the methods comprising exposing the bacteria to an effective amount of any of the compounds, compositions, nucleic acids, vectors, or cells disclosed herein.

Also disclosed herein are methods of reducing bacterial population, the method comprising exposing the bacteria to an effective amount of any of the compounds, compositions, nucleic acids, vectors, or cells disclosed herein.

Also disclosed herein are methods of killing bacteria, the methods comprising exposing the bacteria to an effective amount of any of the compounds, compositions, nucleic acids, vectors, or cells disclosed herein.

Also disclosed herein are methods of treating, preventing, and/or ameliorating a disease or a disorder in a plant or a subject in need thereof, the method comprising administering to the plant or subject a therapeutically effective amount of any of the compounds, compositions, nucleic acids, vectors, or cells disclosed herein.

In some examples, the disease or disorder comprises an infection, such as with an infectious microbe (e.g., bacteria, virus, fungi, protozoa, etc.). In some examples, the disease or disorder comprises a microbial infection.

Also disclosed herein are methods for treating, preventing, inhibiting, and/or ameliorating a microbial infection in a plant or a subject, comprising administering to the plant or subject an effective amount of any of the compounds, compositions, nucleic acids, vectors, or cells disclosed herein.

In some examples, the plant is a crop.

In some examples, the subject is an animal. In some examples, the animal is a companion animal, livestock, research animal, insect, or human.

In some examples, the compound, composition, nucleic acid, or vector is delivered via cultured Streptomyces.

In some examples, the compounds, compositions, nucleic acids, vectors and/or cells can display broad-spectrum antibacterial activity, with strong inhibition against pathogenic microbes.

The methods of treatment of the disease or disorder described herein can further include treatment with one or more additional agents. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

### Pharmaceutical Compositions

Also disclosed herein are pharmaceutical compositions comprising any of the compounds or compositions disclosed herein.

In some examples, the pharmaceutical composition is administered to a subject. In some examples, the subject is an animal. In some examples, the animal is a companion animal, livestock, research animal, insect, or human.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

### Pharmaceutical Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other substances and/or therapies and/or with surgical treatment. These other substances or treatments can be given at the same as or at different times from the compounds or compositions disclosed herein.

In certain examples, compounds and compositions disclosed herein can be locally administered at one or more anatomical sites, such as sites of microbial infection, optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

### Agricultural Compositions, Formulations, and Methods of Administration

Also disclosed herein are agricultural compositions comprising any of the compounds or compositions disclosed herein, and methods of use thereof.

For example, the compound or composition can be applied to vegetation or an area adjacent the vegetation or applied to soil or water to prevent the emergence or growth of vegetation in an amount sufficient to induce an effect, such as an antimicrobial effect. In some embodiments, compounds or compositions are used in an amount sufficient to induce an antimicrobial effect while still showing good crop compatibility.

The present disclosure also relates to formulations of the compositions and methods disclosed herein. In some embodiments, the formulation can be in the form of a single package formulation including any of the compounds disclosed herein. In some embodiments, the formulation can be in the form of a single package formulation including any of the compounds disclosed herein and further including at least one additive. In some embodiments, the formulation can be in the form of a two-package formulation, wherein one package contains any of the compounds disclosed herein and while the other package contains at least one additive. In some embodiments of the two-package formulation, the formulation including any of the compounds disclosed herein and the formulation including at least one additive are mixed before application and then applied simultaneously. In some embodiments, the mixing is performed as a tank mix (i.e., the formulations are mixed immediately before or upon dilution with water). In some embodiments, the formulation including (a) and the formulation including (b) are not mixed but are applied sequentially (in succession), for example, immediately or within 1 hour, within 2 hours, within 4 hours, within 8 hours, within 16 hours, within 24 hours, within 2 days, or within 3 days, of each other.

In some embodiments, the formulation of any of the compounds disclosed herein is present in suspended, emulsified, or dissolved form. Exemplary formulations include, but are not limited to, aqueous solutions, powders, suspensions, also highly-concentrated aqueous, oily or other suspensions or dispersions, aqueous emulsions, aqueous microemulsions, aqueous suspo-emulsions, oil dispersions, self-emulsifying formulations, pastes, dusts, and materials for spreading or granules.

In some embodiments, the compound or composition is an aqueous solution that can be diluted before use. In some embodiments, the compound or composition is provided as a high-strength formulation such as a concentrate. In some embodiments, the concentrate is stable and retains potency during storage and shipping. In some embodiments, the concentrate is a clear, homogeneous liquid that is stable at temperatures of 54° C. or greater. In some embodiments, the concentrate does not exhibit any precipitation of solids at temperatures of −10° C. or higher. In some embodiments, the concentrate does not exhibit separation, precipitation, or crystallization of any components at low temperatures. For example, the concentrate remains a clear solution at temperatures below 0° C. (e.g., below −5° C., below −10° C., below −15° C.). In some embodiments, the concentrate exhibits a viscosity of less than 50 centipoise (50 megapascals), even at temperatures as low as 5° C.

The compositions and methods disclosed herein can also be mixed with or applied with an additive. In some embodiments, the additive can be diluted in water or can be concentrated. In some embodiments, the additive is added sequentially. In some embodiments, the additive is added simultaneously. In some embodiments, the additive is premixed with the compound.

In some embodiments, the additive is an additional pesticide. For example, the compositions described herein can be applied in conjunction with one or more additional pesticides. The composition can be formulated with the one or more additional pesticides, tank mixed with the one or more additional pesticides, or applied sequentially with the one or more additional pesticides.

In some embodiments, the additional pesticide or an agriculturally acceptable salt or ester thereof is provided in a premixed formulation with the compound.

In some embodiments, the additive includes an agriculturally acceptable adjuvant. Exemplary agriculturally acceptable adjuvants include, but are not limited to, antifreeze agents, antifoam agents, compatibilizing agents, sequestering agents, neutralizing agents and buffers, corrosion inhibitors, colorants, odorants, penetration aids, wetting agents, spreading agents, dispersing agents, thickening agents, freeze point depressants, antimicrobial agents, crop oil, herbicide safeners, adhesives (for instance, for use in seed formulations), surfactants, protective colloids, emulsifiers, tackifiers, and mixtures thereof.

Exemplary agriculturally acceptable adjuvants include, but are not limited to, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphate alcohol ethoxylate; natural primary alcohol (C₁₂-C₁₆) ethoxylate or less, di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8 EO); tallow amine ethoxylate (15 EO); and PEG(400) dioleate-99.

In some embodiments, the additive is a safener, which is an organic compound leading to better crop plant compatibility when applied with a pesticide. In some embodiments, the safener itself is herbicidally active. In some embodiments, the safener acts as an antidote or antagonist in the crop plants and can reduce or prevent damage to the crop plants.

Exemplary surfactants (e.g., wetting agents, tackifiers, dispersants, emulsifiers) include, but are not limited to, the alkali metal salts, alkaline earth metal salts and ammonium salts of aromatic sulfonic acids, for example lignosulfonic acids, phenolsulfonic acids, naphthalenesulfonic acids, and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalene sulfonic acids with phenol and formaldehyde, polyoxyethylene octylphenol ether, ethoxylated isooctyl-, octyl- or nonylphenol, alkylphenyl or tributylphenyl polyglycol ether, alkyl aryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g., methylcellulose), hydrophobically modified starches, polyvinyl alcohol, polycarboxylates, polyalkoxylates, polyvinyl amine, polyethyleneimine, polyvinylpyrrolidone and copolymers thereof.

Exemplary thickeners include, but are not limited to, polysaccharides, such as xanthan gum, and organic and inorganic sheet minerals, and mixtures thereof.

Exemplary antifoam agents include, but are not limited to, silicone emulsions, long-chain alcohols, fatty acids, salts of fatty acids, organofluorine compounds, and mixtures thereof.

Exemplary antimicrobial agents include, but are not limited to, bactericides based on dichlorophen and benzyl alcohol hemiformal, and isothiazolinone derivatives, such as alkylisothiazolinones and benzisothiazolinones, and mixtures thereof.

Exemplary antifreeze agents include, but are not limited to ethylene glycol, propylene glycol, urea, glycerol, and mixtures thereof.

Exemplary colorants include, but are not limited to, the dyes known under the names Rhodamine B, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108, and mixtures thereof.

Exemplary adhesives include, but are not limited to, polyvinylpyrrolidone, polyvinyl acetate, polyvinyl alcohol, tylose, and mixtures thereof.

In some embodiments, the additive includes a carrier. In some embodiments, the additive includes a liquid or solid carrier. In some embodiments, the additive includes an organic or inorganic carrier. Exemplary liquid carriers include, but are not limited to, petroleum fractions or hydrocarbons such as mineral oil, aromatic solvents, paraffinic oils, and the like or less, vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like or less, esters of the above vegetable oils or less, esters of monoalcohols or dihydric, trihydric, or other lower polyalcohols (4-6 hydroxy containing), such as 2-ethyl hexyl stearate, n-butyl oleate, isopropyl myristate, propylene glycol dioleate, di-octyl succinate, di-butyl adipate, di-octyl phthalate and the like or less, esters of mono, di and polycarboxylic acids and the like, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers and the like, and water as well as mixtures thereof. Exemplary solid carriers include, but are not limited to, silicas, silica gels, silicates, talc, kaolin, limestone, lime, chalk, bole, loess, clay, dolomite, diatomaceous earth, calcium sulfate, magnesium sulfate, magnesium oxide, ground synthetic materials, pyrophyllite clay, attapulgus clay, kieselguhr, calcium carbonate, bentonite clay, Fuller's earth, cottonseed hulls, wheat flour, soybean flour, pumice, wood flour, walnut shell flour, lignin, ammonium sulfate, ammonium phosphate, ammonium nitrate, ureas, cereal meal, tree bark meal, wood meal and nutshell meal, cellulose powders, and mixtures thereof.

In some embodiments, emulsions, pastes or oil dispersions can be prepared by homogenizing the compound in water by means of wetting agent, tackifier, dispersant or emulsifier. In some embodiments, concentrates suitable for dilution with water are prepared, comprising the compound, a wetting agent, a tackifier, and a dispersant or emulsifier.

In some embodiments, powders or materials for spreading and dusts can be prepared by mixing or concomitant grinding of the compound and optionally a safener with a solid carrier.

In some embodiments, granules (e.g., coated granules, impregnated granules and homogeneous granules) can be prepared by binding the compound to solid carriers.

The compositions disclosed herein can be applied in any known technique for applying pesticides. Exemplary application techniques include, but are not limited to, spraying, atomizing, dusting, spreading, or direct application into water (in-water). The method of application can vary depending on the intended purpose. In some embodiments, the method of application can be chosen to ensure the finest possible distribution of the compositions disclosed herein.

If desired, the compositions can be applied as an in-water application.

When the compositions are used in crops, the compositions can be applied after seeding and before or after the emergence of the crop plants. In some embodiments, when the compositions are used in crops, the compositions can be applied before seeding of the crop plants.

In some embodiments, the compositions disclosed herein are applied to vegetation or an area adjacent the vegetation or applied to soil or water by spraying (e.g., foliar spraying). In some embodiments, the spraying techniques use, for example, water as carrier and spray liquor rates of from 10 liters per hectare (L/ha) to 2000 L/ha (e.g., from 50 L/ha to 1000 L/ha, or from 100 to 500 L/ha). In some embodiments, the compositions disclosed herein are applied by the low-volume or the ultra-low-volume method, wherein the application is in the form of micro granules. In some embodiments, wherein the compositions disclosed herein are less well tolerated by certain crop plants, the compositions can be applied with the aid of the spray apparatus in such a way that they come into little contact, if any, with the leaves of the sensitive crop plants while reaching the undesirable population or the bare soil (e.g., post-directed or lay-by). In some embodiments, the compositions disclosed herein can be applied as dry formulations (e.g., granules, WDGs, etc.) into water.

The compositions and methods disclosed herein can also be used in plants that are resistant to, for instance, pesticides, pathogens, and/or insects. In some embodiments, the compositions and methods disclosed herein can be used in plants that are resistant to one or more pesticides because of genetic engineering or breeding.

In some embodiments, the compositions described herein and other complementary pesticides are applied at the same time, either as a combination formulation or as a tank mix, or as sequential applications.

The compositions and methods may be used in controlling undesirable populations in crops possessing agronomic stress tolerance (including but not limited to drought, cold, heat, salt, water, nutrient, fertility, pH), pest tolerance (including but not limited to insects, fungi and pathogens) and crop improvement traits (including but not limited to yield; protein, carbohydrate, or oil content; protein, carbohydrate, or oil composition; plant stature and plant architecture).

The herbicidal compositions described herein can be used to control herbicide resistant or tolerant populations. The methods employing the compositions described herein may also be employed to control herbicide resistant or tolerant populations. Exemplary resistant or tolerant populations include, but are not limited to, biotypes with resistance or tolerance to multiple herbicides, biotypes with resistance or tolerance to multiple chemical classes, biotypes with resistance or tolerance to multiple herbicide modes-of-action, and biotypes with multiple resistance or tolerance mechanisms (e.g., target site resistance or metabolic resistance).

The present compositions may be formulated and delivered to host plants by methods known in the art, including soil drench via soil drench formulations, seed inoculation via seed inoculation formulations, and plant inoculation via plant inoculation formulations. Seed inoculation formulations can include a carrier such as peat slurry or a film coat consisting of alginate polymers, to protect the compositions from environmental stresses such as desiccation and temperature perturbations. Soil drench or in-furrow composition delivery to plants may be performed by applying the compositions and/or composition formulations in soil before or after planting. Soil drench has several advantages over seed inoculation: 1) prevents the compositions or composition formulations from being inhibited by the chemicals coated on seeds (e.g., fungicides and pesticides) and 2) delivers compositions or composition formulations at higher density without being constrained by seed size. A higher composition or composition formulation concentration is usually required for soil inoculation. Foliar spray and root dipping are also suitable for composition or composition formulation delivery of plants. Plants may be treated at the seedling stage to increase persistence in the plant. In addition, seedling priming, direct seed coating, alginate seed coating, and 12-h coating are within the scope of the present disclosure.

The compositions in the present invention may be formulated and administered to insect hives as a liquid suspension, powder, or solid substrates, such as lipid-based patties.

Liquid formulations may optionally comprise water, sugar syrup and/or other carbohydrate, vitamins, stabilizers, and any other nutrients supportive of bee health. Dry formulations may optionally comprise powdered sugar or other carbohydrate, vitamins, stabilizers, and any other nutrients supportive of bee health. Patty formulations may comprise sugar and/or other carbohydrate, vegetable and/or animal fat, vitamins, stabilizers, and any other nutrients supportive of bee health.

The compositions may be administered as a treatment and/or prophylactically. The compositions may also be administered as a protocol that includes vaccination, phage therapy, the use of lactic acid-producing bacteria.

The formulations optionally include additional foulbrood treatments, such as tylosin tartrate (produced by Elanco, e.g., tylosin A, B, C, and D), and/or Terramycin® (produced by Pfizer, e.g. TM25®, TM50@, TM100@), including Terra-Pro®, and/or the active ingredient of Terramycin®, oxytetracycline HCL. For example, the compounds and compositions disclosed herein can be formulated and/or used in conjunction with the known foulbrood treatments. Therefore, the methods include treatment with one or more of the present compositions and can optionally include additional treatments from previously-known modalities.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

Described herein are genetic and enzymatic methods to produce and obtain phosphonopyruvate, phosphonoalanine and phosphonoalanine-peptide compounds using engineered strains and recombinant protein catalysts developed from Streptomyces microorganisms. These compounds are valuable chemical synthons, neuroactive agents, research reagents, and have potential application as antimicrobial agents against plant and animal pathogens of human and agricultural concern. Potent inhibition of bacterial pathogens was previously demonstrated using the phosphonoalanine containing di- and tripeptides, specifically against the causative agents of soft rots and pink seed disease. These compounds also inhibit bacterial pathogens attributed to the devasting collapse of honeybees worldwide.

Also described are the generation of a broad series of dipeptides using one of the catalysts. Dipeptides have applications as flavoring agents, to address nutritional deficiencies, and other medical relevant activities including hypertension and depression.

Herein methods are reported for utilizing the purified enzymes as effective biocatalysts for these compounds in both individual and combinatorial reactions. Their utility in phosphonopeptide synthesis shown by rapidly producing all previously described phosphonoalamide compounds (six) and generating new peptides not previously observed in Nature.

### Example 2—Convergent Biosynthesis and Diversification of Phosphonoalanine Oligopeptide Natural Products in Streptomyces

ABSTRACT. Phosphonate natural products, with their potent inhibitory activity, have found widespread use across medicine and biotechnology. Their history of success has inspired development of genome mining approaches that continue to reveal previously unknown bioactive phosphonate scaffolds and biosynthetic insights. However, a greater understanding of phosphonate metabolism is required to refine discovery attempts by improving the prediction of compounds and their bioactivities from sequence information alone. Here, knowledge of this natural product class is expanded by reporting the complete biosynthetic pathway for the phosphonoalamides, tripeptides with a conserved N-terminal L-phosphonoalanine (PnAla) residue produced by Streptomyces. The phosphonoalamides result from the convergence of PnAla biosynthesis and peptide ligation pathways. The biochemistry underlying the transamination of phosphonopyruvate to PnAla, a new early branchpoint in phosphonate biosynthesis catalyzed by an aspartate aminotransferase which has evolved with specificity for phosphonate metabolism, is elucidated. Peptide formation was shown to be catalyzed by two ATP-grasp ligases, the first of which produces dipeptides, and a second which ligates said dipeptides to PnAla to produce the phosphonoalamides. Substrate specificity profiling revealed a dramatic expansion of observed dipeptide and tripeptide products, with the ligase PnaC to be the most promiscuous dipeptide ligase reported thus far. These findings highlight previously unknown transformations in natural product biosynthesis, promising enzyme biocatalysts, and unveil insights into the potential diversity of phosphonopeptide natural products which remain to be discovered.

INTRODUCTION. Phosphonate and phosphinate (Pn) compounds are characterized by their direct C—P bonds. This moiety, which enables chemical mimicry of phosphate esters and carboxylic acids, is responsible for the bioactivity of these compounds [B1]. The majority of phosphonate and phosphinate natural products (NPs) are potent metabolic inhibitors, with a historical commercialization rate over two order of magnitudes greater than natural products as a whole [B2]. These compounds have found broad success, with examples including the antibiotic fosfomycin (Monurol), the antiviral phosphonoformate (Foscarnet), and the antimalarial fosmidomycin. Fosfomycin mimics phosphoenolpyruvate (PEP) to covalently inhibit MurA, blocking peptidoglycan biosynthesis [B3]. Foscarnet is a pyrophosphate analog which inhibits viral polymerases [B4, B5]. Lastly, fosmidomycin blocks the non-mevalonate pathway of isoprenoid biosynthesis by inhibiting 1-deoxy-D-xylulose 5-phosphate reductoisomerase, and has shown promising results against malaria in human trials [B6].

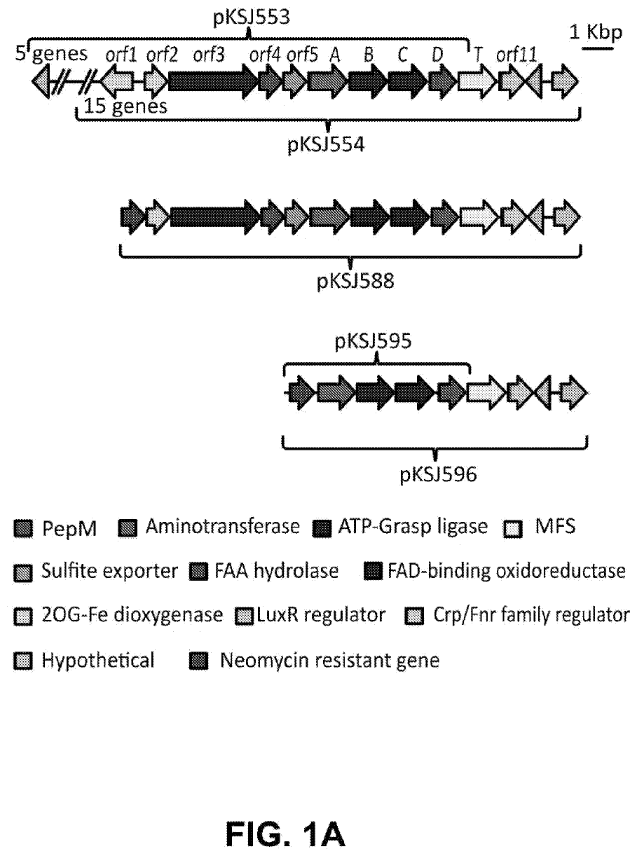

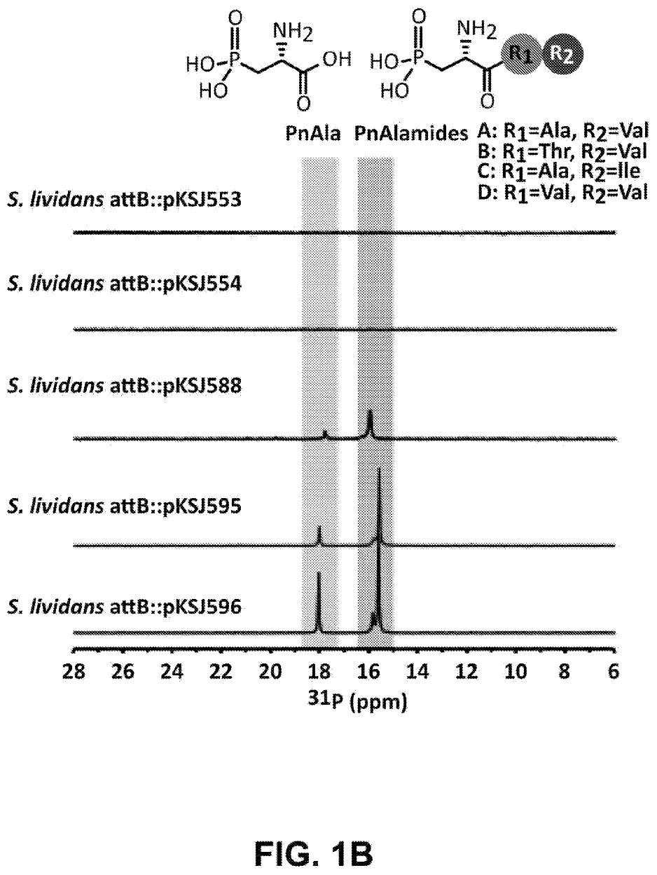

This established track record for phosphonate and phosphinate natural products has inspired genomics-driven methods that have enabled the discovery of multiple, diverse, antimicrobial and herbicidal phosphonate and phosphinate scaffolds [B2, B7-B13]. Even with these findings, genomic data suggest a tremendous wealth of unexplored chemical diversity remains, as nearly 7% of all bacteria are predicted to encode biosynthetic pathways for phosphonate and phosphinate natural products [B14]. In spite of this, a greater understanding of these biosynthetic gene clusters (BGCs) is required to improve the discovery process, enabling accurate prediction and classification of their pathways, products, and biological activities. While all known phosphonate and phosphinate biosynthesis begins with the isomerization of PEP to phosphonopyruvate (PnPy) by phosphoenolpyruvate mutase (PepM), this reaction is highly unfavorable, and requires a coupled enzymatic reaction to provide the thermodynamic energy to drive it forward [B15]. This principle was leveraged to identify a family of biosynthetic gene clusters encoding a previously unknown coupling enzyme, resulting in the isolation of L-phosphonoalanine (PnAla) and four tripeptides with an N-terminal PnAla (phosphonoalamides A-D) from Streptomyces sp. NRRL B-2790 (FIG. 1B) [B8].

Although PnAla was the second Pn compound ever isolated from biological material [B16], the role it plays in Nature still remains unknown six decades later. In experimental physiology, PnAla is used within cerebral tissues as a selective antagonist of metabotropic glutamate receptors and an inhibitor of phosphoserine phosphatases [B17-B19]. PnAla has been found within multiple tissues including the human liver, intestine, and spleen [B20], and genes implicated in PnAla degradation are found across several classes of bacteria [B21]. The isolation of strains capable of using free PnAla as a sole carbon, nitrogen, and phosphorus source suggest it, like other phosphonate and phosphinate compounds, may play a role in global nutrient cycles [B21-B25]. However, PnAla had not been observed within Nature as a component of a small molecule or free amino acid until the previously reported isolation of PnAla and its peptide conjugants [B8, B9]. These studies also revealed their biological activity as broad-spectrum antibacterials, suggesting roles in microbial chemical ecology beyond that of a mere nutrient [B8, B9].

Despite its frequent isolation from biological macromolecules and widespread use in research, several aspects of PnAla metabolism remain uncharacterized, including the nature of the transamination resulting in its biosynthesis and rationale for how this reaction thermodynamically drives unfavorable C—P bond formation [B1, B8]. The mechanism by which PnAla is incorporated into peptides also remains unknown, though conservation of biosynthetic genes between the Streptomyces (N-terminal PnAla) and Bacillus (C-terminal PnAla) biosynthetic gene clusters suggest ATP-grasp ligases are responsible for amide bond formation, with their differential specificities driving diversification of these peptides [B8, B9]. The existence of additional related biosynthetic gene clusters among terrestrial, marine, and plant-associated bacteria, as well as the differing spectrums of activity between phosphonoalamide A (PnAla-Ala-Val) and phosphonoalamide F (Ala-Ala-PnAla), further underscore the diversity and potential of PnAla-containing natural products [B9].

Here, knowledge of phosphonate and phosphinate metabolism is advanced by establishing the genetic and biochemical logic for phosphonoalamide biosynthesis in Streptomyces. The biosynthetic gene cluster was defined using heterologous expression and deletion analyses, with biochemical reconstitution demonstrating phosphonoalamide A to be the product of four enzymatic reactions. Among these steps, the PLP-dependent transamination of PnPy to PnAla by PnaD represents a new early branchpoint in phosphonate and phosphinate metabolism, analogous to the reaction catalyzed by aspartate aminotransferase (AAT). Tripeptide formation was revealed to be a convergent process catalyzed by two distinct ATP-grasp L-amino acid ligases. PnaC produced a diverse array of dipeptides, which were then ligated to PnAla by PnaB. The natural specificity of these ligases was leveraged to generate a total of 181 dipeptides and 97 PnAla-tripeptides, of which 93 had not been previously observed in Nature.

### Results and Discussion

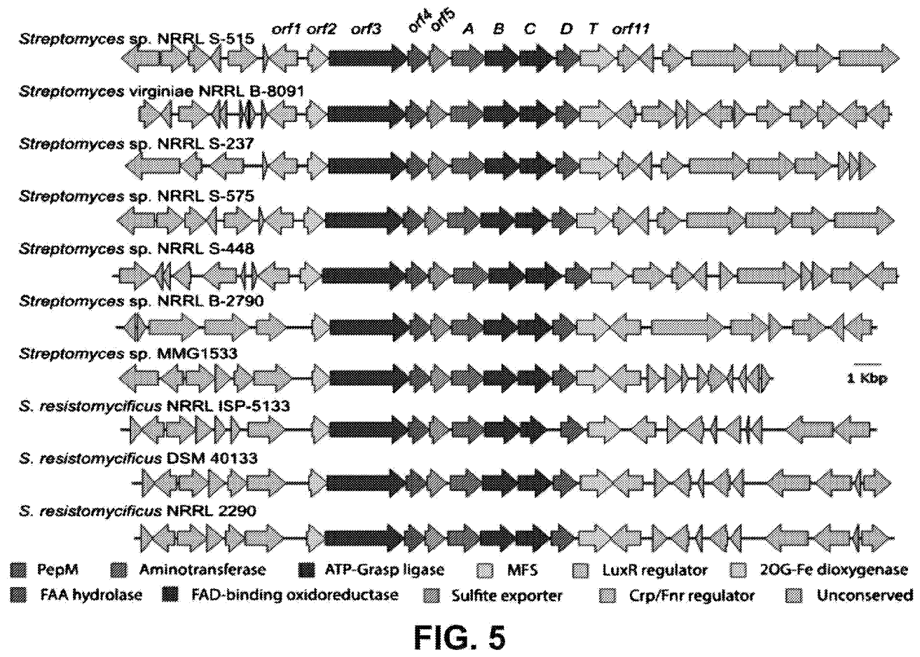

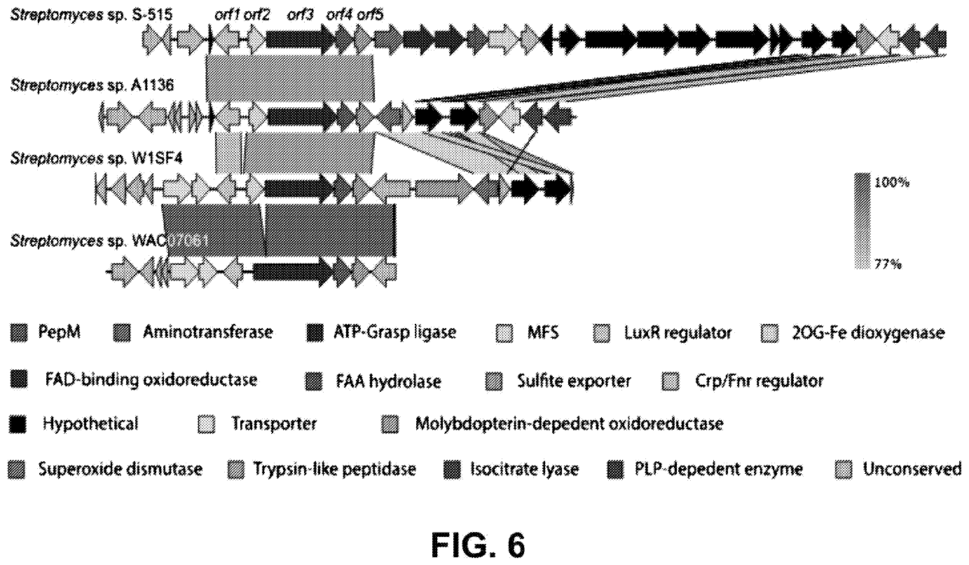

Delineation of the phosphonoalamide biosynthetic gene cluster. All characterized pathways for phosphonate and phosphinate biosynthesis begin with the isomerization of phosphoenolpyruvate (PEP) to 3-phosphonopyruvate (PnPy) by phosphoenolpyruvate mutase (PepM). PepMs with at least 80% sequence identity have been shown to produce similar phosphonate and phosphinate natural products [B7], a fact which was previously used to identify a group of Streptomyces strains producing PnAla and the phosphonoalamides [B8]. As these strains produced the same compounds, it was reasoned that the biosynthetic gene cluster would comprise genes within the pepM neighborhood which were conserved between strains. To provide contigs of sufficient length for synteny analysis, the genomes of Streptomyces sp. NRRL B-2790 and S-488 were re-sequenced. In addition to pepM (designated as pnaD), clear conservation was observed for genes encoding a putative transcriptional regulator (orf1), two oxidoreductases (orf2 and orf3), a hydrolase (orf4), a transaminase (pnaA), two ligases (pnaB and pnaC), and two transporters (orf5 and pnaT) (FIG. 5; Table 1). Except for orf1 (putative FAA hydrolase) and orf11 (Crp/Fnr transcriptional regulator), gene arrangement was largely conserved across all 10 strains. Breaks in synteny beyond orf12 and orf11 suggested these as the boundaries of the biosynthetic gene cluster. However, orf1-5 were present in several genomic neighborhoods of Streptomyces unrelated to phosphonate and phosphinate biosynthesis, suggesting they were not involved (FIG. 6).

TABLE 1
Annotation of the S-515 pepM neighborhood

| | | Amino | | | |
|---|---|---|---|---|---|
| Gene | Accession | acids | NCBI annotation | PFam annotation | CDD Annotation |
| orfl | WP_158820658 | 347 | Helix-turn-helix | Bacterial regulatory | HTH superfamily |
| | | | transcriptional | proteins, LuxR family | (cl21459) |
| | | | regulator | (PF00196) | |
| orf2 | WP_051781700 | 245 | 20G-Fe dioxygenase | 20G-Fe dioxygenase | 20G-Fe_Oxy_2 |
| | | | family protein | (PF10014) | superfamily |
| | | | | | (cl01794) |
| orf3 | WP_030764880 | 963 | FAD-binding | FAD linked oxidases, | GlcC/GlcD |
| | | | oxidoreductase | C-terminal domain | superfamilies |
| | | | | (PF02913) | |
| orf4 | WP_030764879 | 252 | Fumarylacetoacetate | Fumarylacetoacetate | MhpD superfamily |
| | | | hydrolase family | hydrolase family | |
| | | | protein | (PF01557) | |
| orf5 | WP_030764877 | 240 | Sulfite exporter | Sulfite exporter TauE | TauE superfamily |
| | | | TauE/SafE family | (PF01925) | (cl21514) |
| | | | protein | | |
| pnaA | WP_051781701 | 410 | Pyridoxal phosphate- | Aminotransferase | Aspartate |
| | | | dependent | class I and II | aminotransferase |
| | | | aminotransferase | (PF00155) | superfamily |
| | | | | | (cl18945) |
| pnaB | WP_030764872 | 431 | ATP-grasp domain- | ATP-grasp domain | Arginosuccinate lyase |
| | | | containing protein | (PF13535) | superfamily |
| | | | | | (cl35175) |
| pnaC | WP_051781702 | 412 | ATP-grasp domain- | ATP-grasp domain | Arginosuccinate lyase |
| | | | containing protein | (PF13535) | superfamily |
| | | | | | (cl35175) |
| pnaD | WP_030764868 | 292 | Phosphoenolpyruvate | Phosphoenolpyruvate | TIM superfamily |
| | | | mutase family protein | phosphomutase | (cl21457) |
| | | | | (PF13714) | |
| pnaT | WP_030902602 | 427 | MFS transporter | Major Facilitator | MFS superfamily |
| | | | | Superfamily | (cl28910) |
| | | | | (PF07690) | |
| orf11 | WP_234440912 | 262 | Crp/Fnr family | Crp-like helix-turn- | Crp superfamily |
| | | | transcriptional | helix domain | |
| | | | regulator | (PF13545) | |
| orf12 | WP_030764862 | 168 | Hypothetical protein | Family of unknown | DUF5706 superfamily |
| | | | | function (DUF5706) | (cl40967) |
| orf13 | WP_158820660 | 279 | Hypothetical protein | Family of unknown | MSCRAMM_ClfA |
| | | | | function (DUF5545) | superfamily |
| | | | | | (cl41352) |

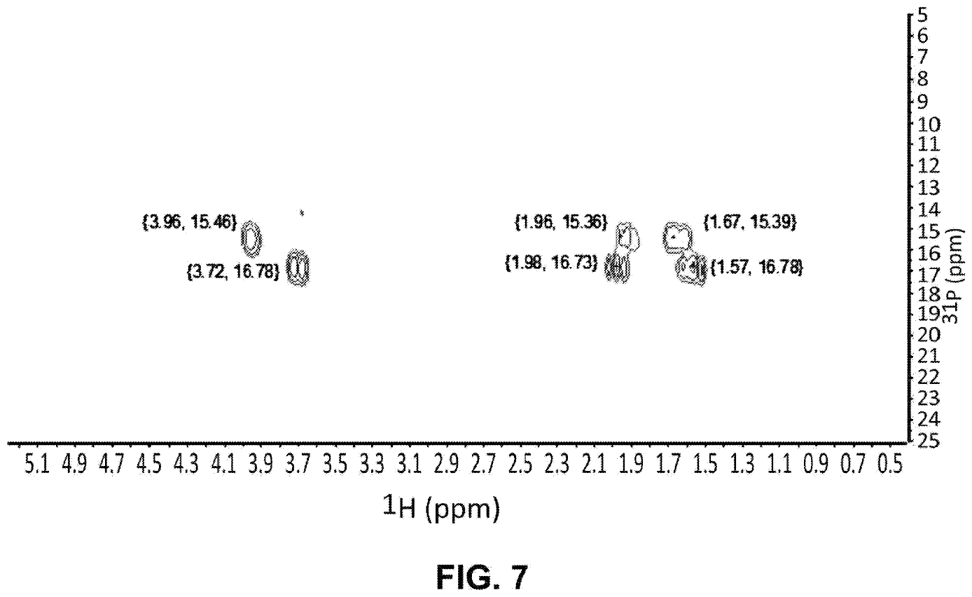

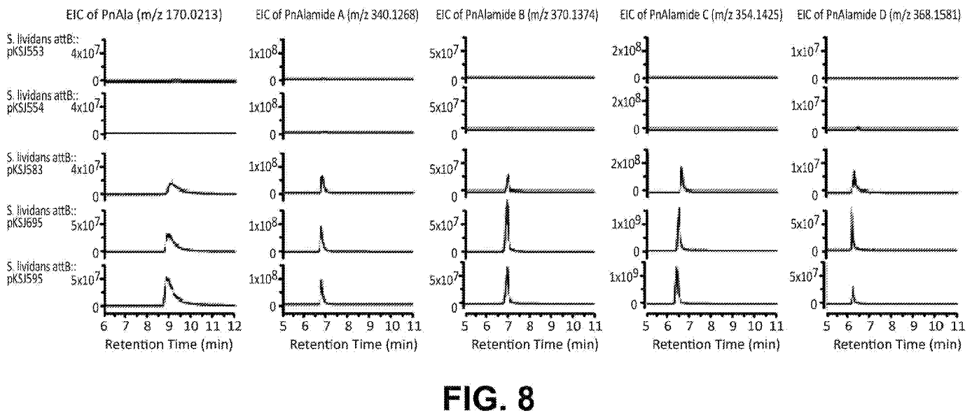

To clearly establish the biosynthetic gene cluster boundaries, heterologous expression and deletion analysis of the gene neighborhood were performed. First, a fosmid library of Streptomyces sp. NRRL S-515 was constructed, two clones encoding overlapping regions of the pnaD neighborhood (pKSJ553 and pKSJ554) were selected, and then they were integrated into the ΦC31 attB site of S. lividans 66. Metabolites produced by the resulting strains (S. lividans 66 attB::pKSJ553, S. lividans 66 attB::pKSJ554) were analyzed by ³¹P NMR and LC-HRMS and compared to a negative integration control strain (S. lividans 66 attB::pAE4). Even after growth on multiple media types, Pns were not observed from either heterologous expression strain. It was reasoned that the lack of production may have been due to inadequate gene expression, possibly stemming from regulation afforded by the adjacently encoded LuxR (orf1) and FNR-type (orf11) transcriptional regulators. To test this hypothesis, λ-Red mediated recombination was used to replace orf1 and upstream genes from S-515 on clone pKSJ554 with an aph resistance cassette, resulting in pKSJ688 (FIG. 1A). This deletion significantly improved Pn production, as evidenced by the multiple Pn signals observed within S. lividans 66 attB::pKSJ588 extracts (FIG. 1i). ³¹P and ¹H-³¹P HMBC spectra matched established literature values for PnAla and phosphonoalamides A-D (FIG. 1B, FIG. 7) [B8] and LC-HRMS unambiguously verified the production of these compounds (FIG. 8).

The biosynthetic gene cluster was further truncated to determine if orf2-5, pnaT, and orf11 are required for Pn biosynthesis. All 6 genes were deleted to create pKSJ595 while only orf2-5 were deleted to create pKSJ596. Interestingly, analysis of both S. lividans 66 attB::pKSJ595 and S. lividans 66 attB::pKSJ596 by ³¹P NMR and LC-HRMS revealed an identical profile of Pn species produced, but with greater abundance than in S. lividans 66 attB::pKSJ588. These results were consistent with synteny analyses and clearly demonstrate pnaABCD as the only genes required for phosphonoalamide biosynthesis.

A dedicated phosphonopyruvate transaminase for L-PnAla biosynthesis. Having established the minimal biosynthetic gene cluster, attention was focused on the biosynthesis of PnAla and its peptide derivatives. Previous co-expression of pnaD and pnaA in S. albus J1074 resulted in production of PnAla [B8], suggesting that PnaA functions as a coupling enzyme for PepM to drive the formation of PnPy. PnaA was identified as a pyridoxyl-5-phospate (PLP) dependent aminotransferase belonging to the aspartate aminotransferase (AAT) superfamily (Table 1). Canonical AATs catalyze the interconversion of L-Asp and α-ketoglutarate (αKG) to L-Glu and oxaloacetate (OAA) using PLP reaction chemistry [B26]. This, along with clear structural similarities between PnPy and PnAla to OAA and L-Asp, suggested PnaA may have originated as an AAT that subsequently evolved reaction specificity for Pn substrates.

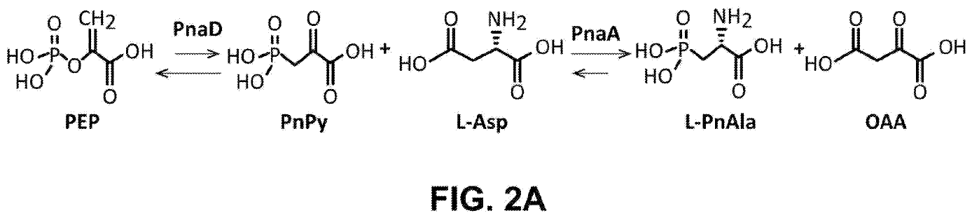

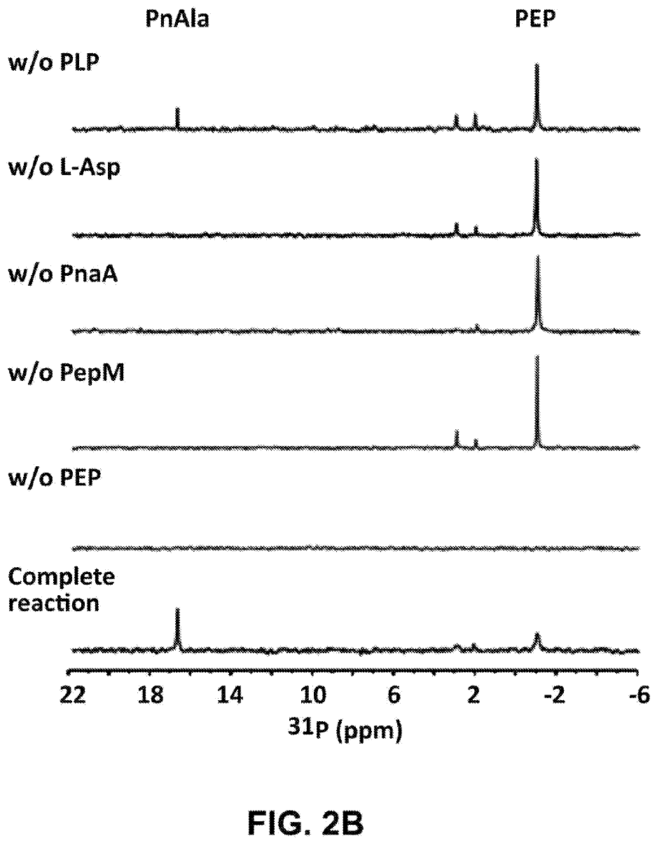

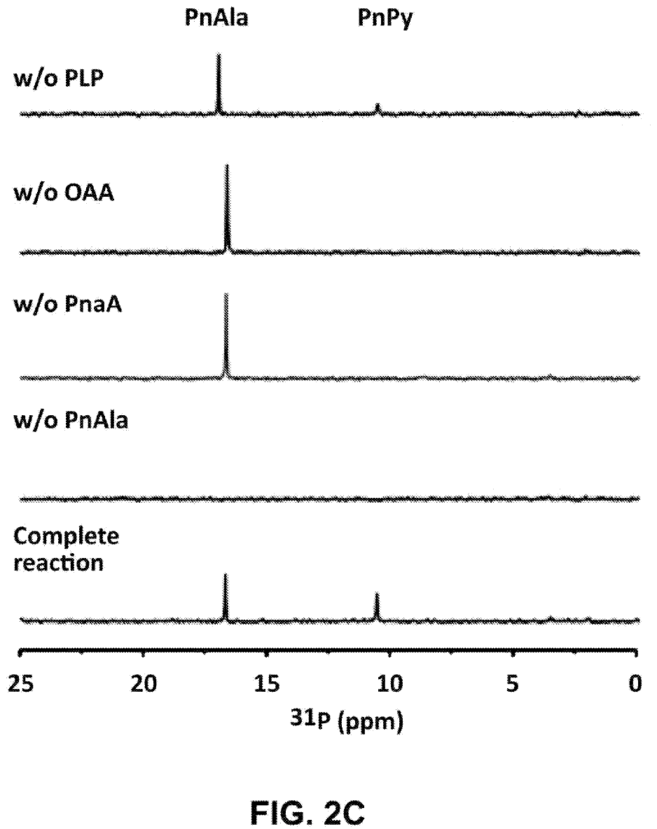

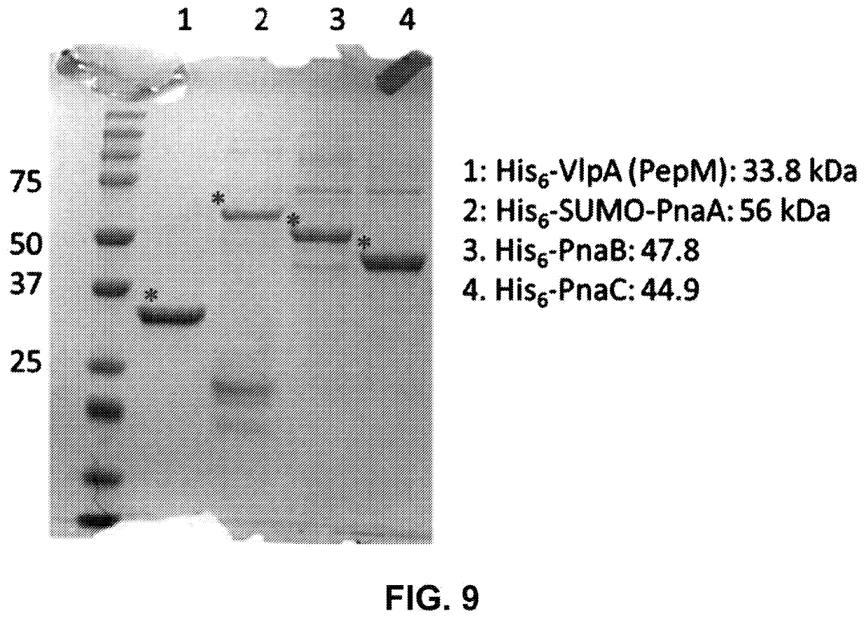

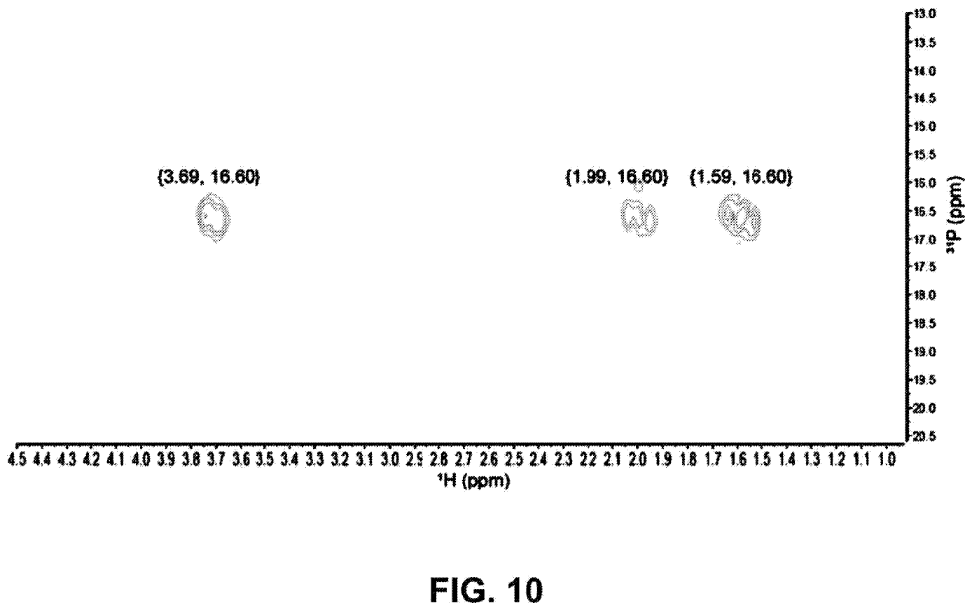

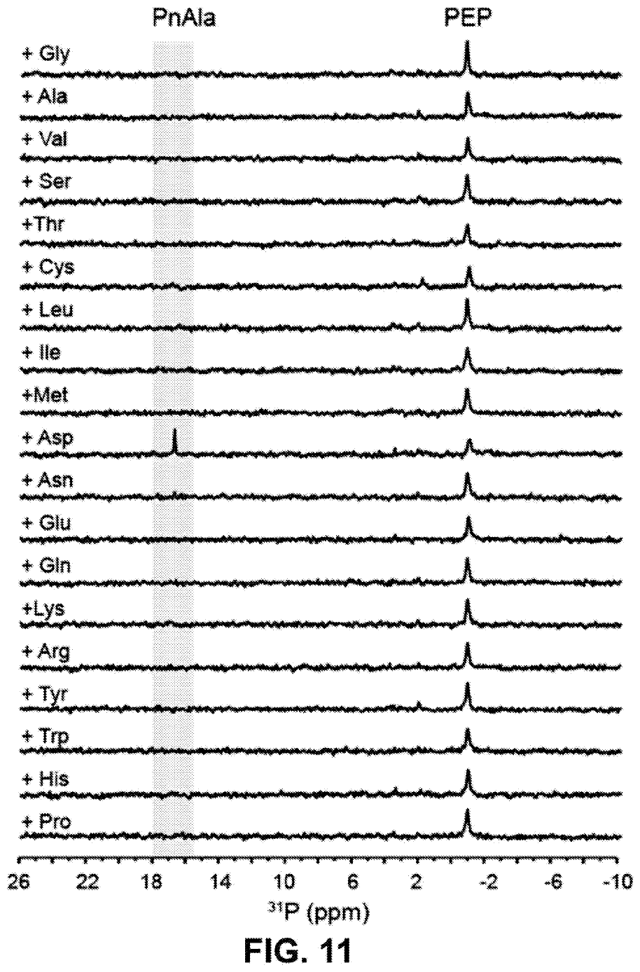

To test this hypothesis and investigate the biochemical nature of PnAla formation, recombinant PnaD and PnaA were overexpressed and purified from E. coli for in vitro assays (FIG. 9). It was proposed that following isomerization of PEP to PnPy by PnaD, the amine of L-Asp would be transferred onto the keto moiety of PnPy by PnaA to yield PnAla and OAA (FIG. 2A). Indeed, incubating PnaA with PnaD, PEP, Asp, and PLP resulted in consumption of PEP (−1.5 ppm) and emergence of a new species (17.3 ppm) in the ³¹P NMR spectra (FIG. 2B). ¹H-³¹P HMBC experiments identified the new signal as PnAla (FIG. 10). Excluding any reaction component other than PLP eliminated PnAla production. Without addition of PLP, enzyme activity was observed at significantly reduced amounts, likely due to endogenous cofactor that co-purified with PnaA. Unlike other aminotransferases, PnAla formation by PnaA was strictly dependent on L-Asp as the amino donor and substitution with other proteinogenic amino acids was not tolerated (FIG. 11).

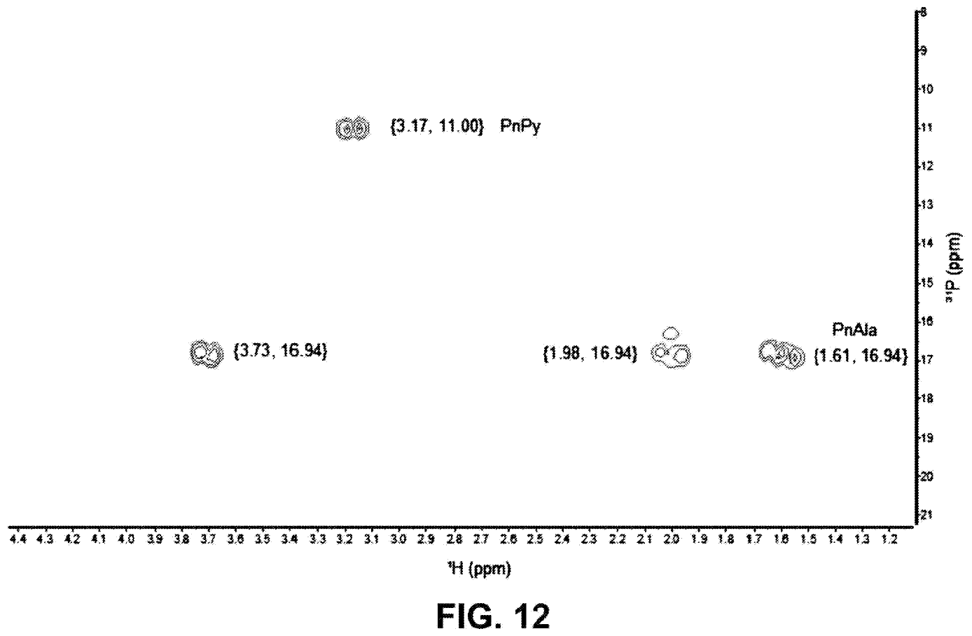

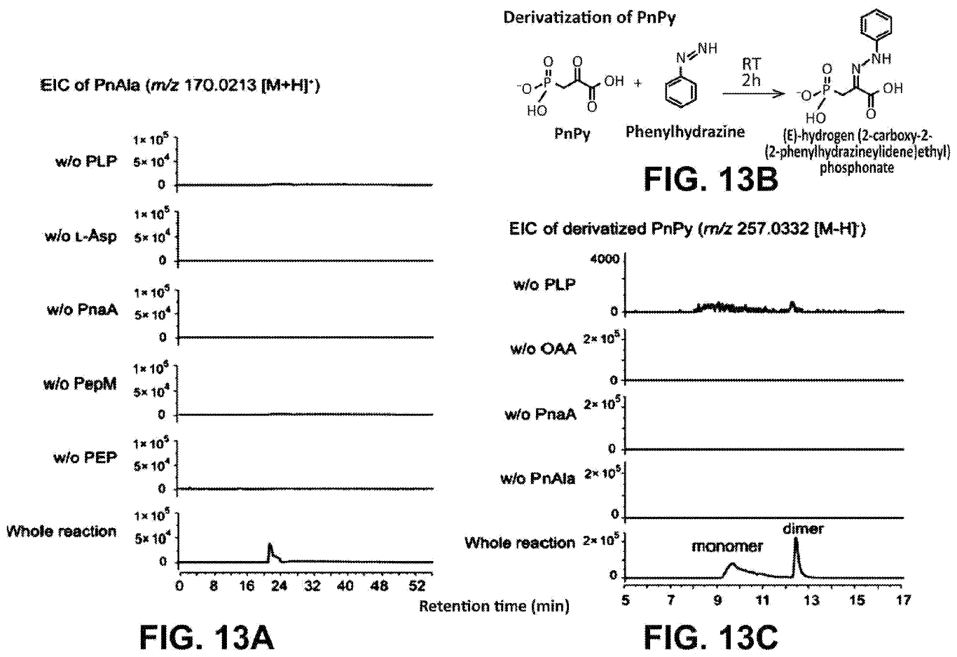

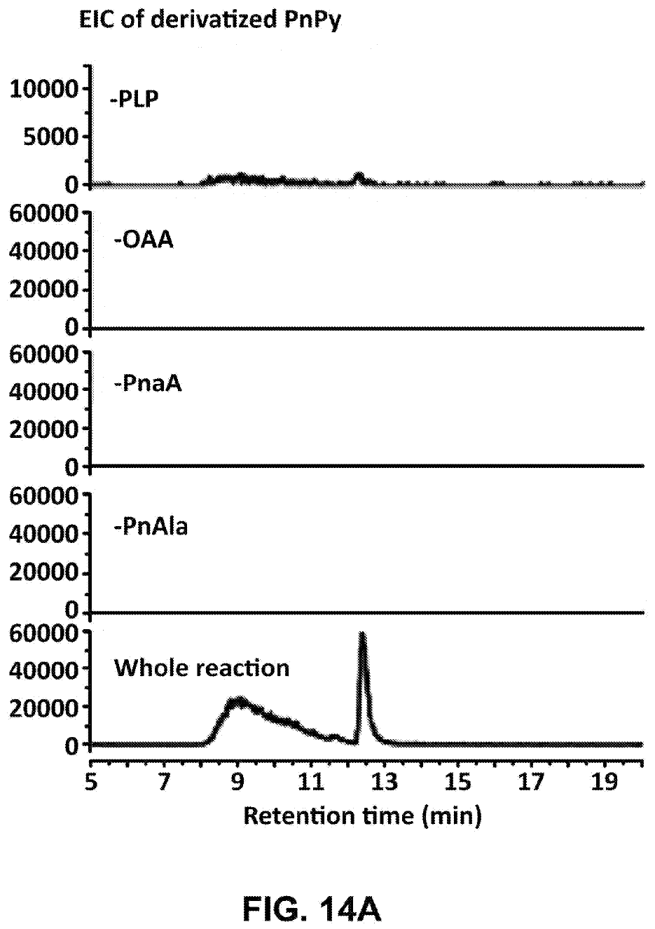

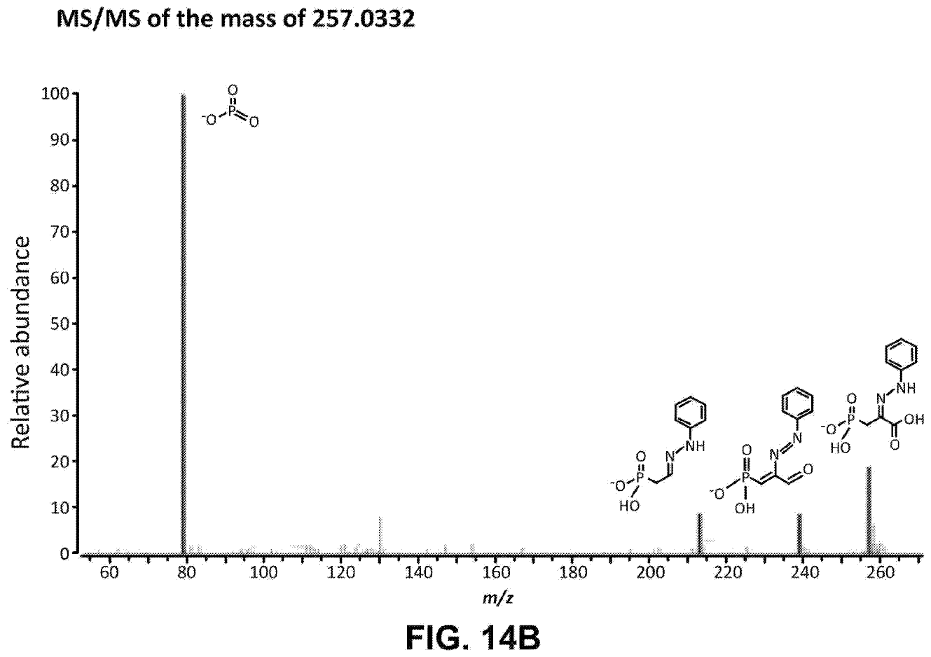

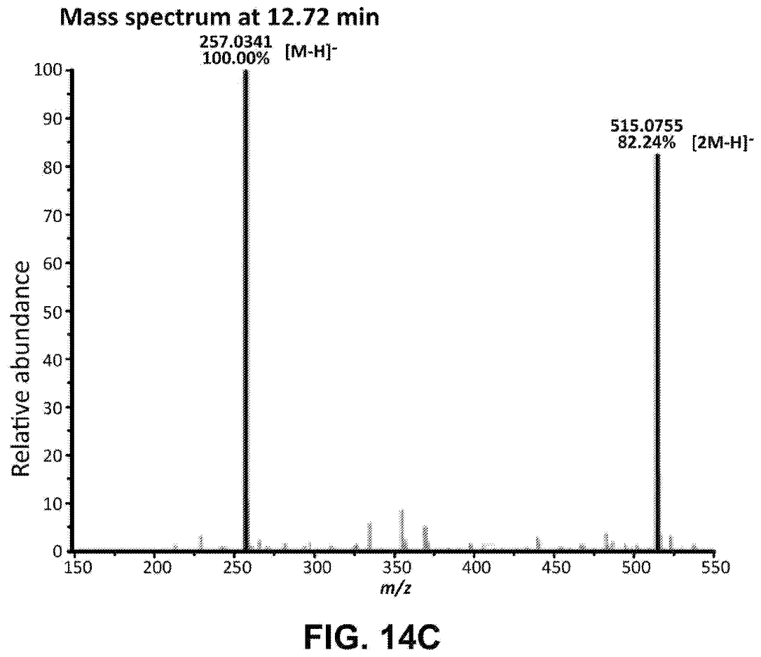

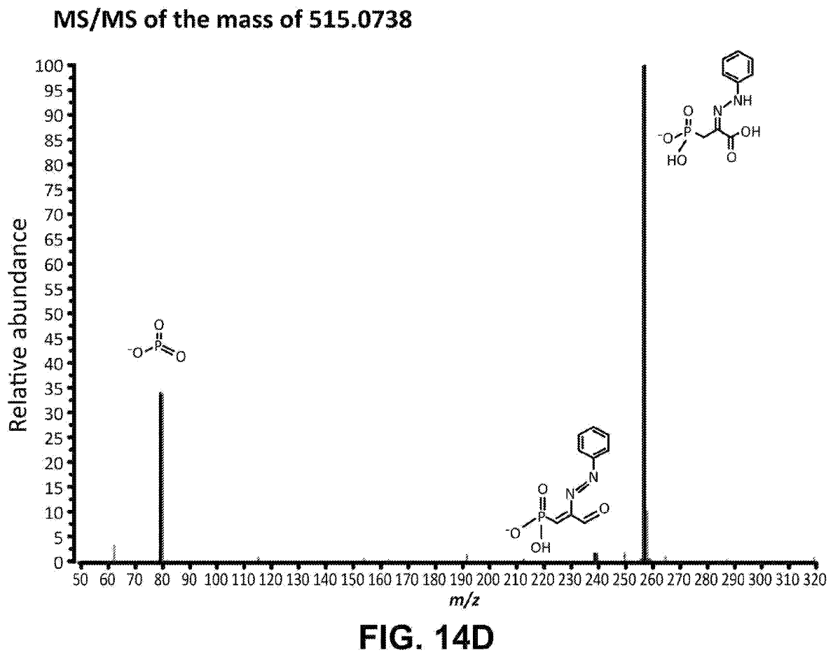

Having established general reaction conditions for PnaA, its ability to catalyze the reverse reaction, transamination of OAA to L-Asp using L-PnAla as an amino donor, was examined. Incubating PnaA with PnAla, OAA, and PLP resulted in a new species in ³¹P NMR (δP=11.0) with ¹H-³¹P HMBC spectra showing correlating protons at 3.17 ppm (FIG. 2C; FIG. 12) [B15]. These properties were consistent with PnPy. To validate its identity, the new species was derivatized with phenylhydrazine to improve its stability and detectability by LC-HRMS. Indeed, both monomeric [M-H]⁻ (257.0332 m/z) and dimeric [2M-H]⁻ (515.0738 m/z) forms of the phenylhydrazine-PnPy conjugate were readily observed, with MS/MS fragmentation confirming their identities (FIG. 13A-FIG. 13C, FIG. 14A-FIG. 14D). The reverse reaction was likewise dependent on all components and significantly greater PnPy was observed when additional PLP was provided (FIG. 2C; FIG. 14A-FIG. 14D).

These results demonstrated PnaA as a PLP-dependent transaminase that functions as a coupling enzyme with PepM. While other PepM coupling enzymes transform PnPy using reactions that are either irreversible (decarboxylation [B27, B28] and acetylation [B29]) or utilize the co-oxidization of NAD(P)H to drive reduction forward [B10], many transamination reactions are naturally reversible under physiological conditions. Indeed, for PnaA the yield of PnAla (50%) from the PepM-coupled forward reaction was comparable to the yield of PnPy (48%) from the reverse reaction (FIG. 2B & FIG. 2C). Nonetheless, it was reasoned that PnaA must have inherent characteristics and reaction conditions that facilitate its function in PnAla biosynthesis.

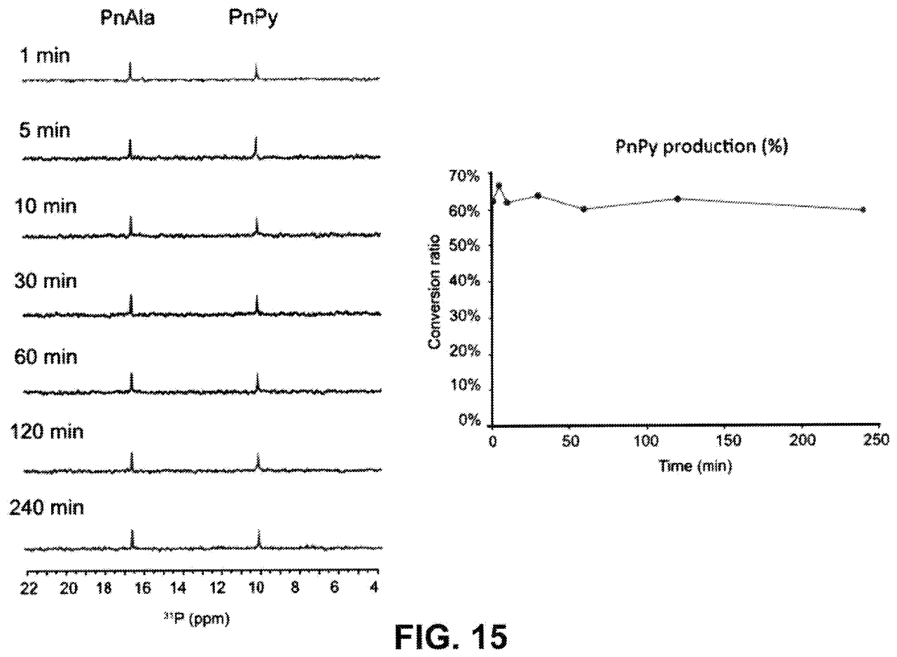

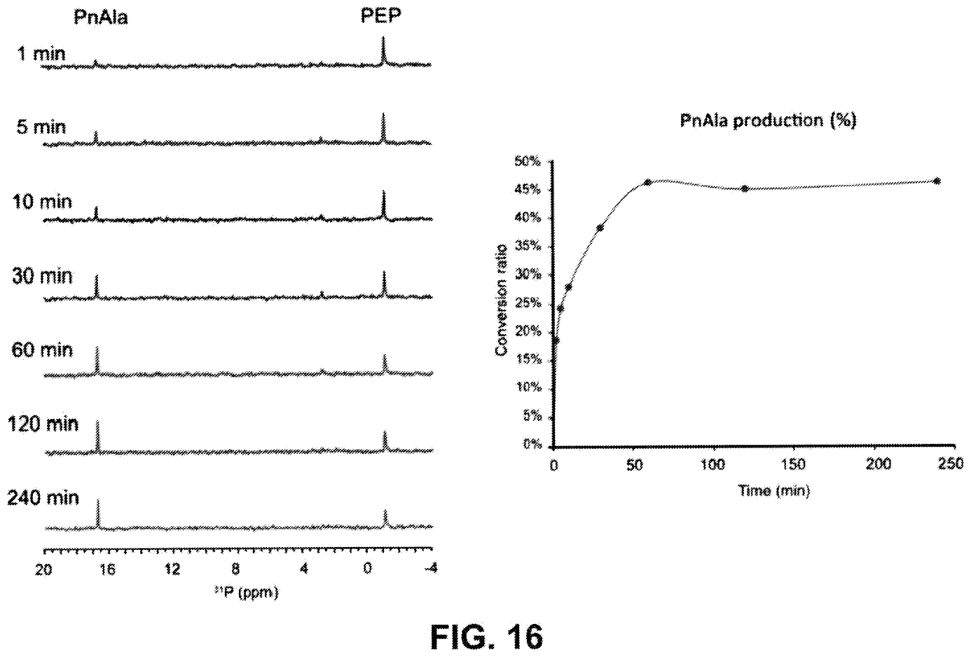

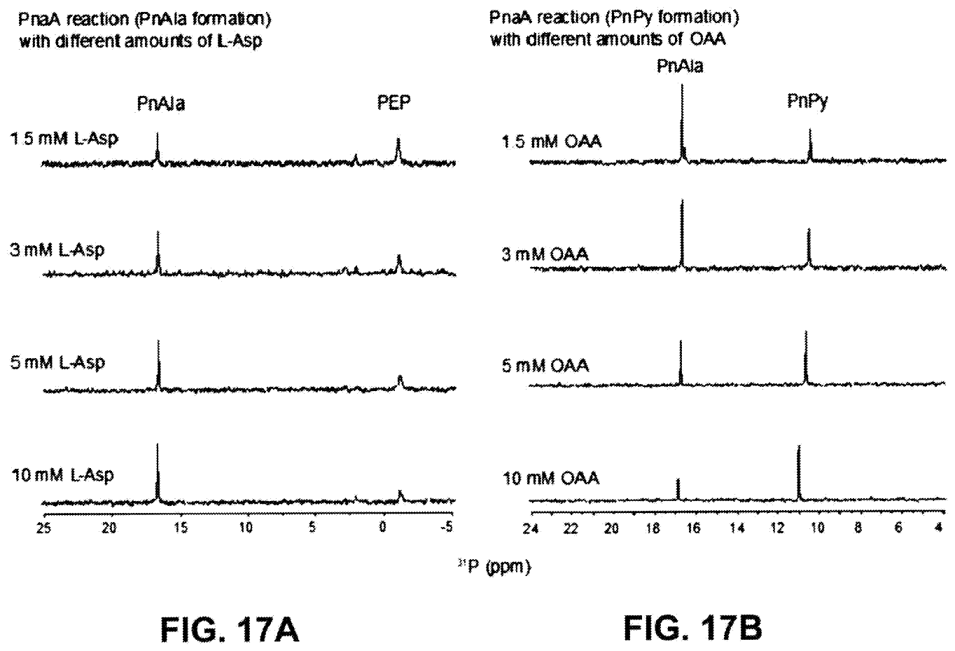

Since all enzymatic transaminations require an amino donor and keto-acid acceptor, a starting point was examining the effects of substrate concentration on the directionality of the PnaA reaction. First, forward and reverse transamination reactions were monitored using ³¹P NMR spectroscopy to understand the general dynamics of PnaA activity over time. Rapid conversion was observed when PnaA was provided a large excess (10 mM) of amino donor (Asp) or keto-acid acceptor (OAA), reaching an apparent equilibrium within two hours (FIG. 15-FIG. 16). Using these conditions, the starting concentration of Asp or OAA were varied (1.5, 3, 5, or 10 mM) in forward and reverse reactions while keeping concentrations of PEP and PnAla constant (1.5 mM) and product formation was measured by ³¹P NMR after 2 hours (FIG. 17A-FIG. 17B). Interestingly, product formation reflected the availability of Asp and OAA co-substrates, accumulating upwards of 70% PnAla or PnPy in their respective reactions. These results indicated that the initial concentration of available amino donors and acceptors strongly influences the degree of product formation at apparent equilibrium.

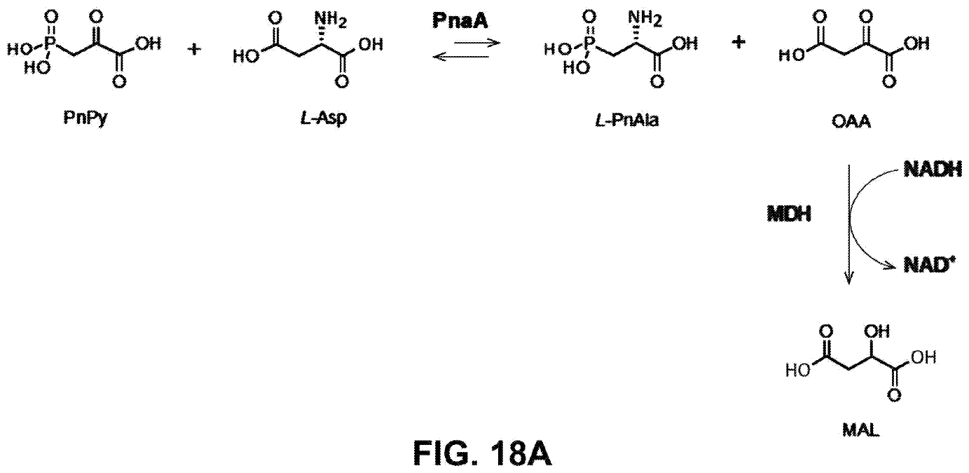

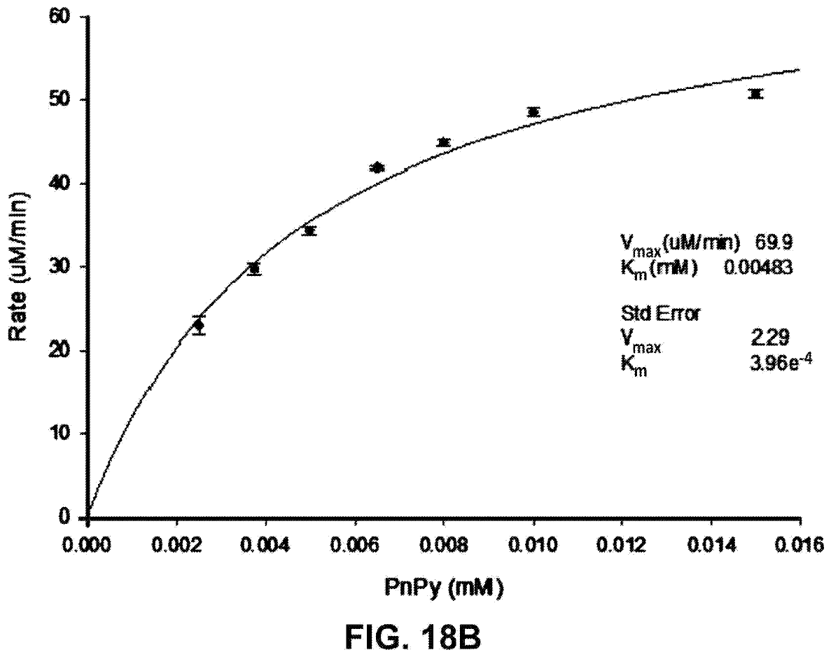

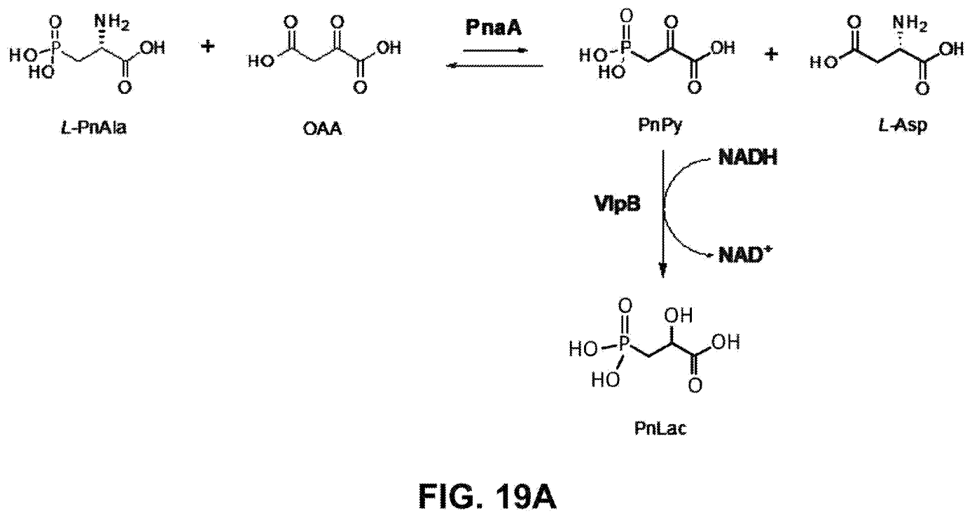

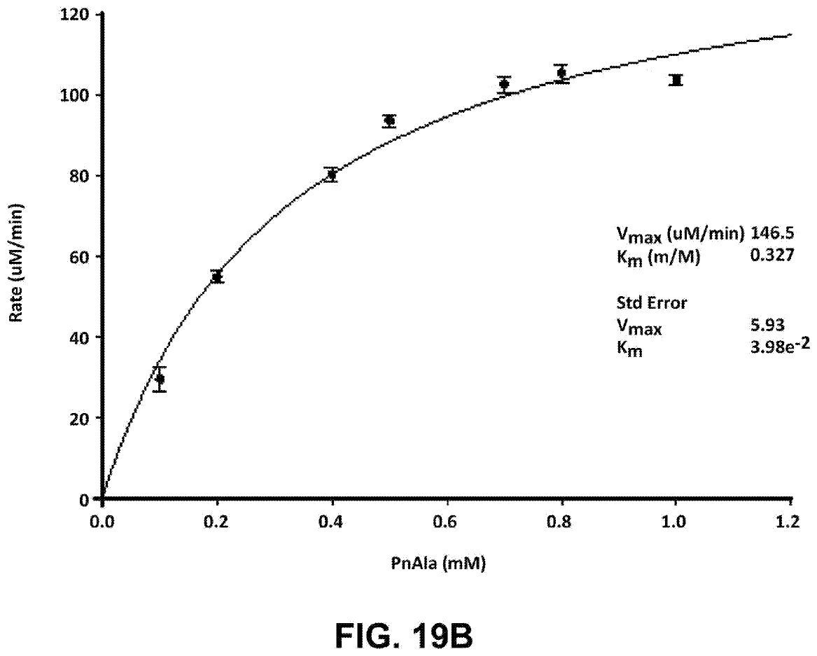

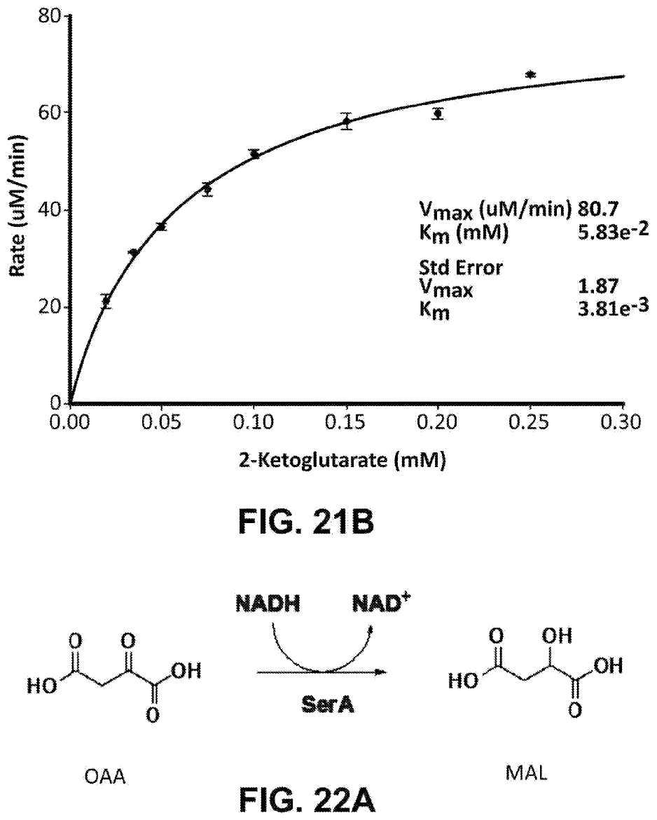

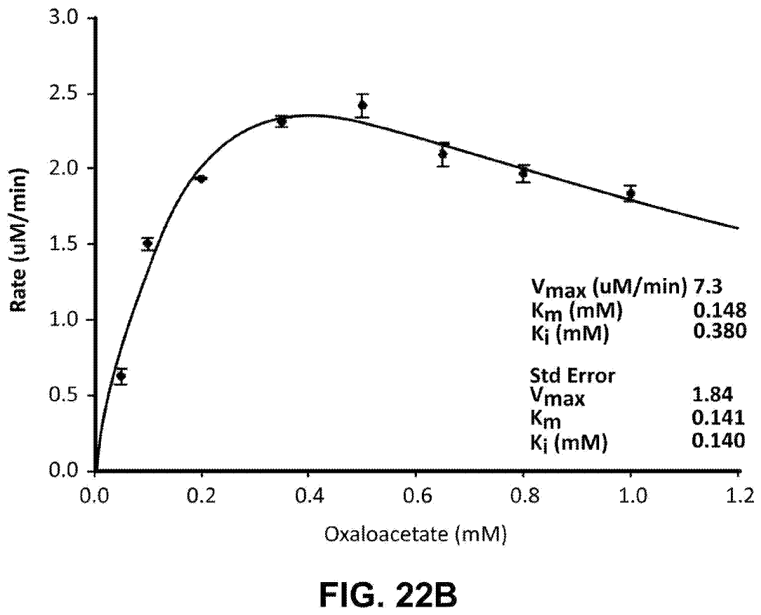

Next, kinetic analyses were performed to derive insight on the substrate specificity and reaction selectivity of PnaA transamination. Apparent steady state parameters were calculated in spectrophotometric assays by coupling the formation of OAA, PnPy, and 2KG with NADH oxidation in the presence of malate dehydrogenase (MDH), PnPy reductase (VlpB), and 3-phosphoglycerate dehydrogenase (SerA), respectively (FIG. 18A-FIG. 24B). The high catalytic efficiency observed with the conversion of Asp and PnPy into OAA and PnAla (k_{cat}=2.3 s⁻¹, Kₘ=0.0048 mM, k_{cat}/Kₘ=4.85×10⁵) further support that this conversion is the physiological reaction catalyzed by PnaA (FIG. 18A-FIG. 18B). The micromolar Kₘ value observed for PnaA with PnPy is similar to that of other PepM coupling enzymes such as PnPy decarboxylase [B27, B30]. In comparison, the reverse reaction (transamination of PnAla to PnPy) exhibited a 100-fold higher Michaelis constant and 100-fold lower catalytic efficiency (k_{cat}=2.4 s⁻¹, Kₘ=0.327 mM, k_{cat}/Kₘ=7.47×10³) (FIG. 19A-FIG. 19B).

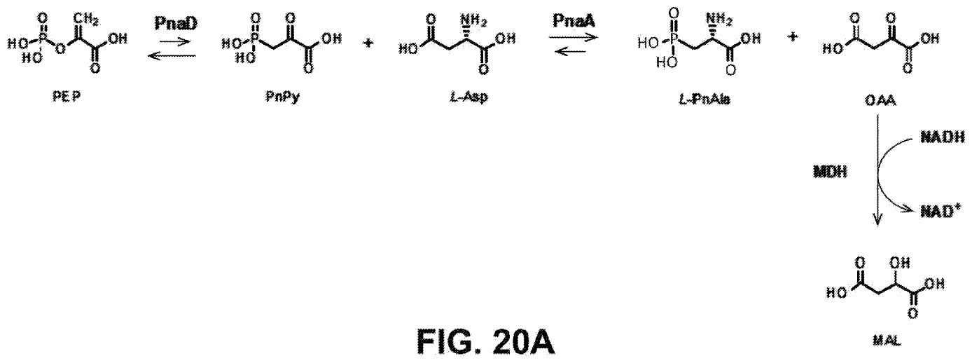

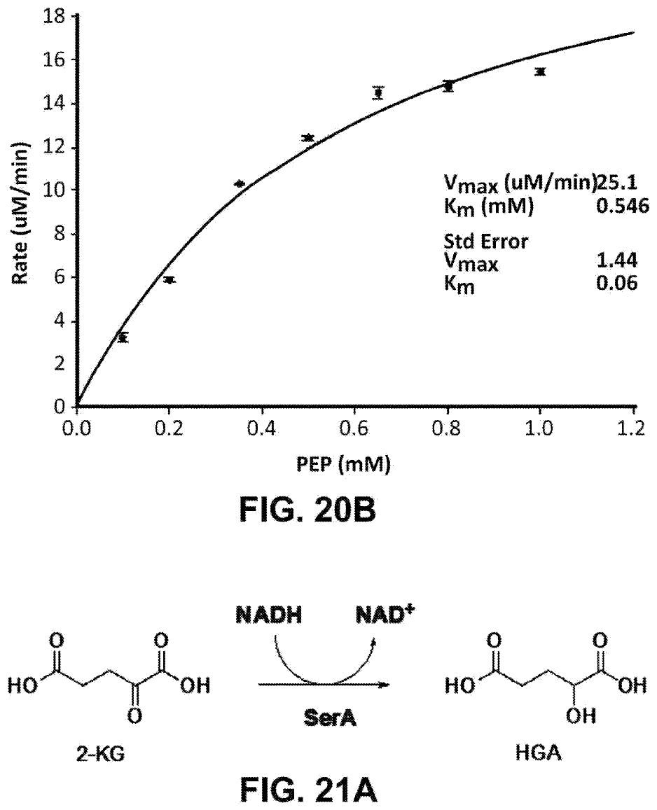

Apparent steady state parameters were also derived for the conversion of PEP to L-PnAla in reactions with equimolar amounts of PnaD and PnaA (FIG. 20A-FIG. 20B). PnAla formation from PEP using both biosynthetic enzymes exhibited a catalytic efficiency 1,000-fold lower (k_{cat}=0.084 s⁻¹, Kₘ=0.547 mM, k_{cat}/Kₘ=1.53×10²) than that of PnaA directly provided with PnPy. The lower activity in the two-enzyme system is consistent with the slower rate of L-PnAla accumulation observed in the time course experiments (FIG. 16), reflects the unfavourability of PnPy formation [B30], and supports the general hypothesis that PepM activity is the limiting step in Pn biosynthetic pathways.

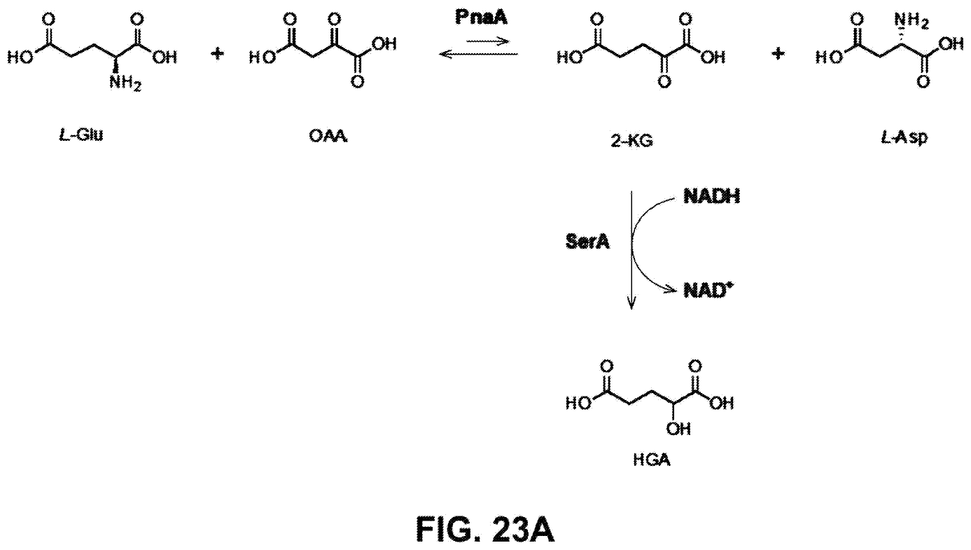

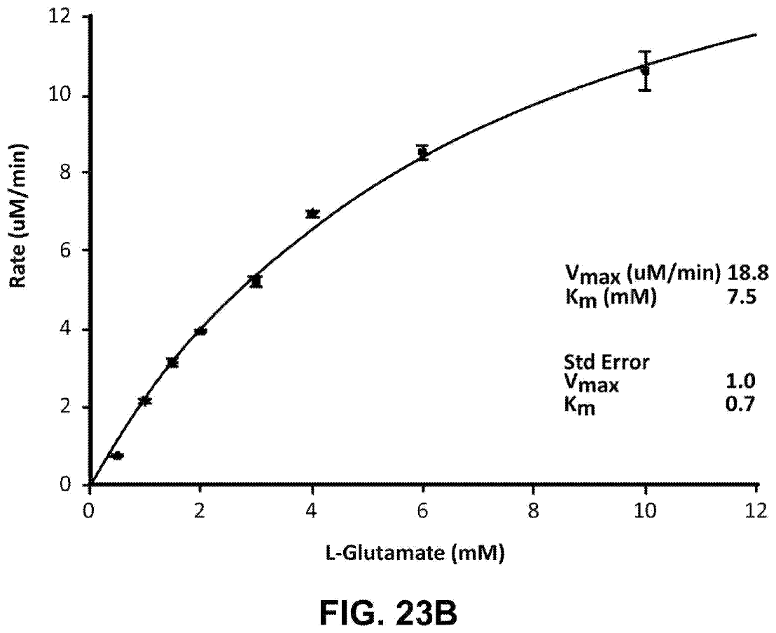

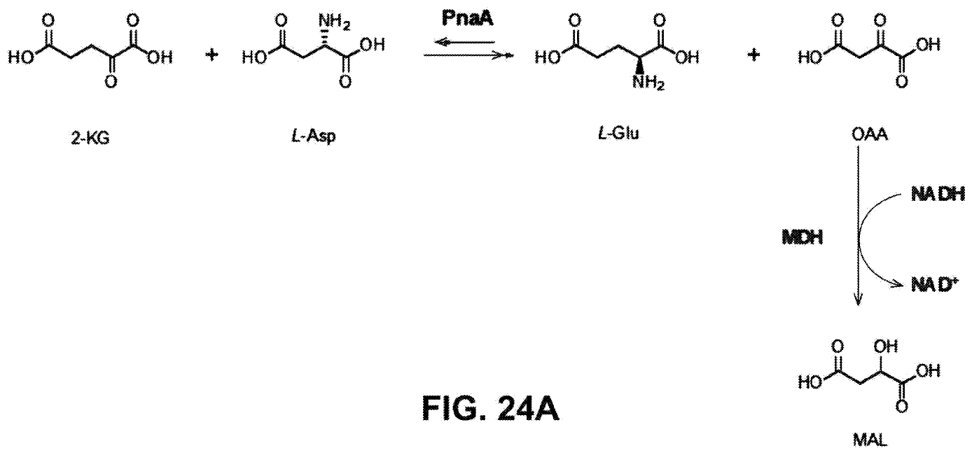

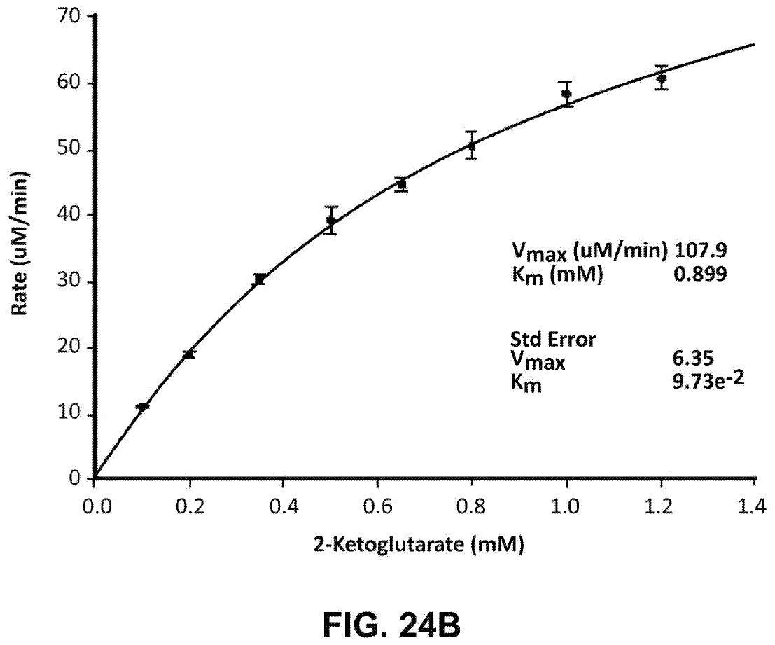

In addition to the transamination of Pn substrates, PnaA was also capable of catalyzing the reversible conversion of Glu and OAA into 2KG and Asp. However, the catalytic efficiencies observed for Glu (k_{cat}=0.31 s⁻¹, Kₘ=7.5 mM, k_{cat}/Kₘ=41.3, FIG. 23A-FIG. 23B) and 2KG transamination reactions (k_{cat}=1.8 s⁻¹, Kₘ=0.899 mM, k_{cat}/Kₘ=2.00×10³, FIG. 24A-FIG. 24B) were manyfold lower than the AAT dedicated for amino acid biosynthesis [B31], and the above reactions with PnPy and PnAla.

These collective results demonstrate PnaA functions as a reversible PLP-dependent transaminase with significant preference for Pn substrates while retaining minor AAT activity. Although substrate concentration influences the degree of product formation at apparent equilibrium, within the timescale of kinetic analyses. PnaA exhibited the greatest affinity for PnPy and the highest catalytic activity in converting PnPy to PnAla, further validating its function as a PepM coupling enzyme.

Phosphonoalamides are formed by convergent biosynthesis. Having established the biochemistry behind PnAla formation, efforts were redirected towards the biosynthetic reactions resulting in oligopeptide formation. The two remaining genes in the biosynthetic gene cluster encoded putative ATP-grasp ligases (pnaB and pnaC), a superfamily of enzymes that catalyze diverse reactions including those within the biosynthesis of amino acids, essential macromolecules, and peptide natural products [B32, B33]. It was therefore hypothesized that PnaB and PnaC may catalyze the final steps in phosphonoloamide biosynthesis by ligating amino acids onto PnAla.

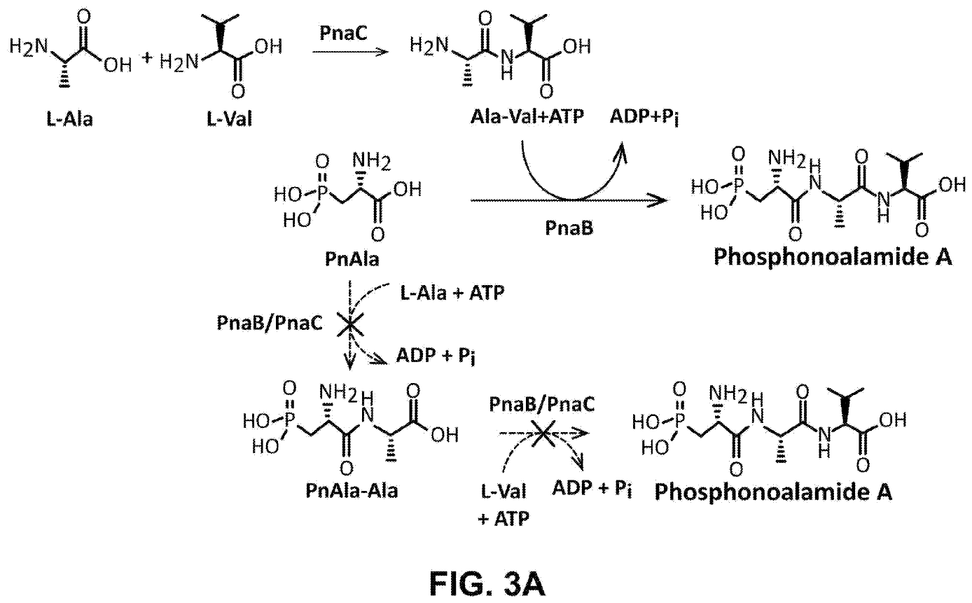

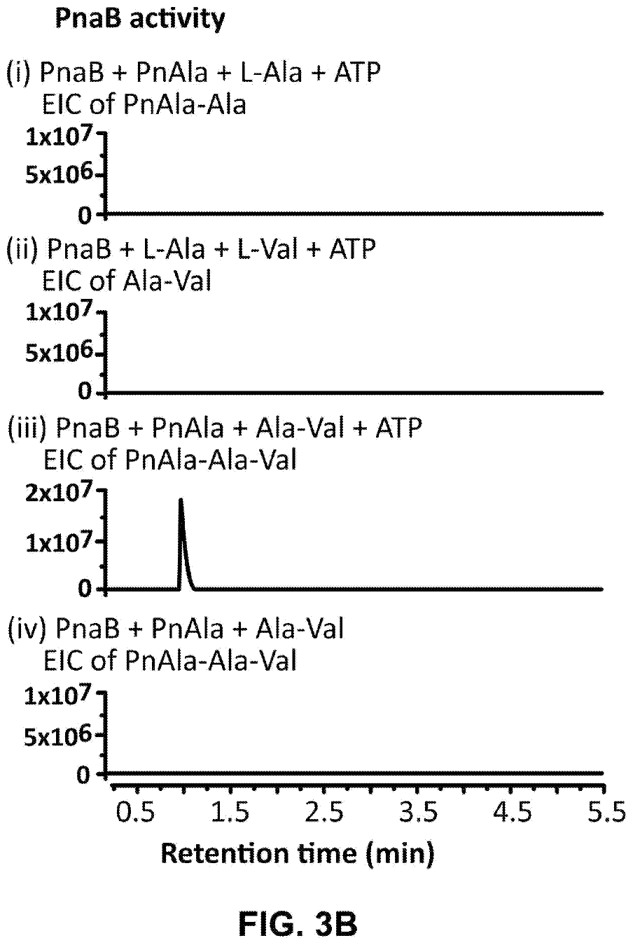

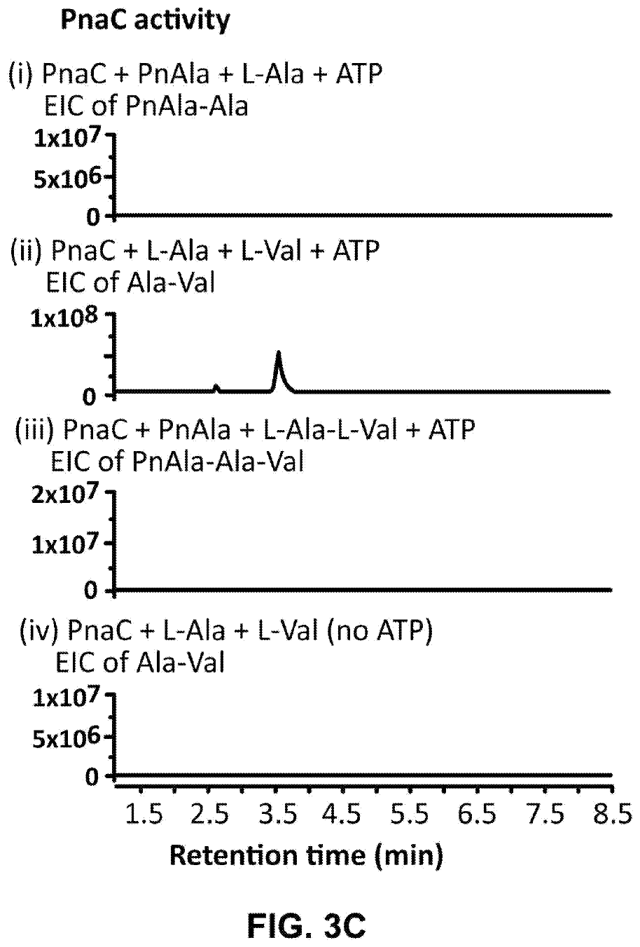

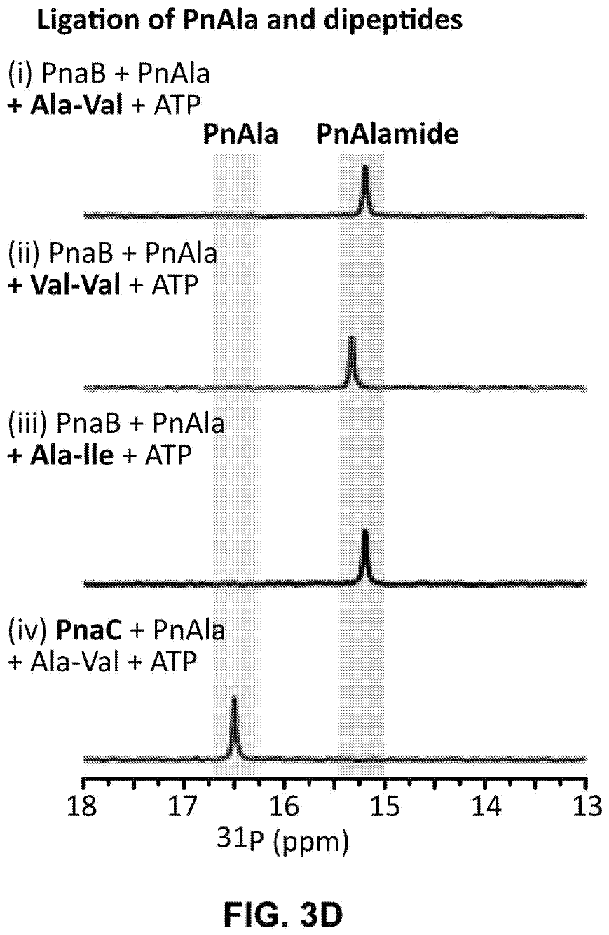

Two possible routes were envisioned by which PnaB and PnaC could function. As phosphonoalamide A (PnAla-Ala-Val) was the most abundant phosphonopeptide from the heterologous expression and native production strains, it was used as the representative product for establishing the pathway. In the first scenario, Ala and Val may be sequentially ligated onto PnAla, with one enzyme responsible for each of the reactions. Invoking the canonical mechanism for peptide bond formation by ATP-grasp ligases, one ligase would activate the carboxylate of PnAla into an acylphosphate intermediate via ATP hydrolysis, priming it for nucleophilic attack by the amine of Ala to form PnAla-Ala. This dipeptide would then be ligated to Val by the remaining ligase, yielding phosphonoalamide A (FIG. 3A). Alternatively, the pathway could begin with one ligase activating Ala to form Ala-Val. Then, PnAla would be activated by the second enzyme and ligated to the dipeptide to produce phosphonoalamide A.

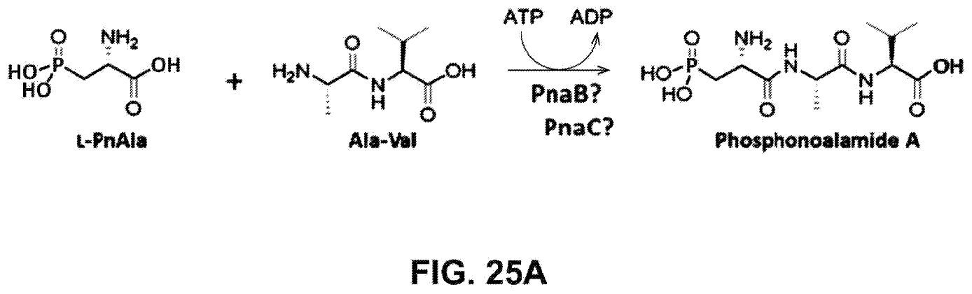

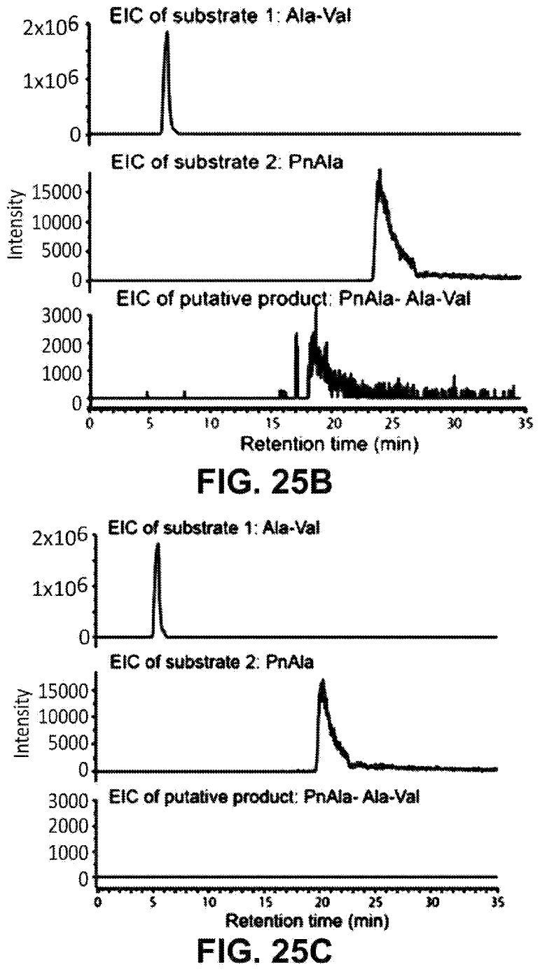

These hypotheses were tested by overproducing and purifying recombinant PnaB and PnaC from E. coli for use in a series of biochemical assays. PnAla, Ala, and ATP were incubated with PnaB or PnaC and the reactions were monitored by ³¹P NMR and LC-HRMS. Even with extended incubation up to 16 hrs, PnAla remained unmodified, indicating neither enzyme forms the PnAla-Ala dipeptide (FIG. 3Bi and FIG. 3Ci). After substituting PnAla with Val, LC-HRMS identified Ala-Val as a product of the PnaC reaction (FIG. 3Cii), with no product observed with PnaB (FIG. 3Bii). To determine if either enzyme could convert Ala-Val into phosphonoalamide A, protein was removed from the PnaC reaction by ultrafiltration and Ala, ATP, and fresh PnaC or PnaB was added to the filtrate. LC-HRMS identified PnAla-Ala-Val as a product following the addition of PnaB, but not PnaC (FIG. 25A-FIG. 25C). As very little phosphonoalamide A was observed, the assays were repeated with individual enzymes, ATP, PnAla, and chemically synthesized Ala-Val to eliminate the effect of competing substrates. ³¹P NMR analysis revealed complete conversion of PnAla into phosphonoalamide A by PnaB, while PnAla remained unmodified by PnaC (FIG. 3D). Incubating PnaB with ATP, PnAla, and chemically synthesized Thr-Val, Ala-Ile, and Val-Val resulted in their respective conversion into phosphonoalamides B, C, and D (FIG. 3D and FIG. 25A-FIG. 25C). ATP was strictly required for the activity of PnaB and PnaC, as its omission abolished all activity (FIG. 3Biv and FIG. 3Civ).

Thus, PnaB and PnaC are essential ATP-grasp ligases with distinct roles in phosphonoalamide formation. These results demonstrate that phosphonoalamide biosynthesis in Streptomyces occurs via a convergent pathway. PnAla is derived from PEP by PnaD and PnaA while PnaC functions as an L-amino acid ligase (LAL) to produce dipeptides. PnaB then serves to ligate PnAla and a dipeptide to form the phosphonoalamides (FIG. 3A).

An overlooked diversity of L-PnAla oligopeptides. Biosynthetic pathways which utilize ATP-grasp ligases for peptide biosynthesis are often capable of producing a mixture of products, including other phosphonopeptide natural products composed of the same Pn headgroup adorned with different amino acids [B8, B10, B34]. These observations, combined with the fact that four unique phosphonoalamides were isolated from Streptomyces, suggested PnaB and PnaC may exhibit relaxed substrate specificity and could yield many more PnAla-oligopeptides than previously identified. With 2 variable positions and 20 proteinogenic amino acids, there exist 400 possible tripeptides with PnAla at the N-terminus. To determine whether additional variants are produced and provide physiological insight into the specificity of these two ligases, the LC-HRMS datasets for S. lividans 66 attB::pKSJ595 were analyzed for possible tripeptide structures.

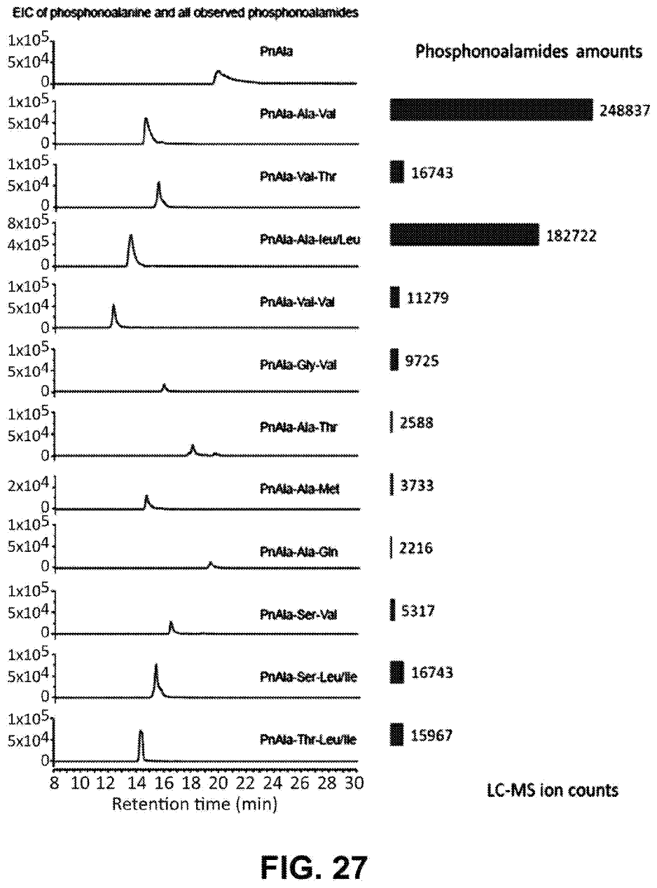

In addition to the isolated phosphonoalamides A-D (PnAla-Ala-Val, PnAla-Thr-Val, PnAla-Ala-Ile, and PnAla-Val-Val respectively), signals corresponding to ten additional tripeptides containing residues which had not been observed before (Gly, Met, Gln, and Ser) were detected (FIG. 27). While these new tripeptides were less abundant than the known phosphonoalamides, these data suggested the dipeptide and tripeptide ligases PnaC and PnaB may naturally accommodate a broader range of substrates than previously realized.

The ATP-Grasp ligases PnaC and PnaB underlie diversification of L-PnAla-containing oligopeptides. To understand the biochemical nature of this potential phosphonopeptide diversity, the substrate specificity of the dipeptide ligase PnaC was first delineated. Microscale biochemical reactions containing purified PnaC were performed with every combination of the 20 canonical amino acids and L-allo-threonine (231 total pairs of substrates). Reactions were analyzed by LC-HRMS to reveal putative dipeptides from 146 out of the 231 combinations. To distinguish residues in reactions containing isomeric pairs (e.g., Ile and Leu, Thr and allo-Thr) new reactions were prepared using ¹⁵N-Ile and 4-¹³C-2,3-D₂-Thr as substrates. LC-HRMS/MS fragmentation analysis was then performed to verify all products and determine structures.

Structure assignment was facilitated by identification of conserved peptide fragment ions, with the y₁ ion enabling assignment of the N-to-C orientation for each dipeptide. For example, a dipeptide composed of Ala and Ile would be either Ala-Ile or Ile-Ala. However, the y₁ fragment would include either protonated Ile or Ala. As these are mutually exclusive, the presence of protonated Ile within the fragmentation pattern of this dipeptide unambiguously assigned it as Ala-Ile. Indeed, all X-Ile dipeptides shared this diagnostic y₁ ion.

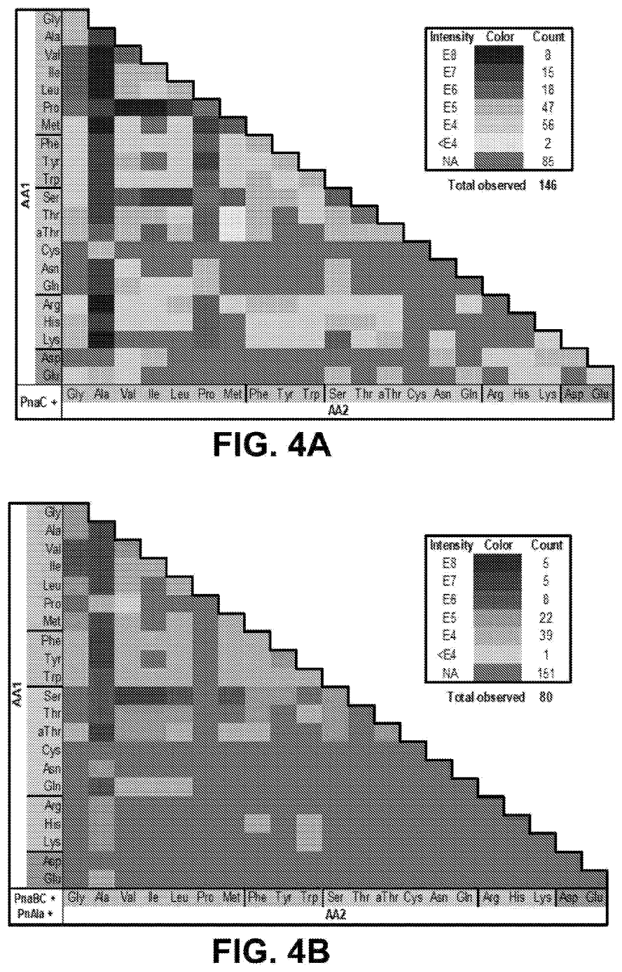

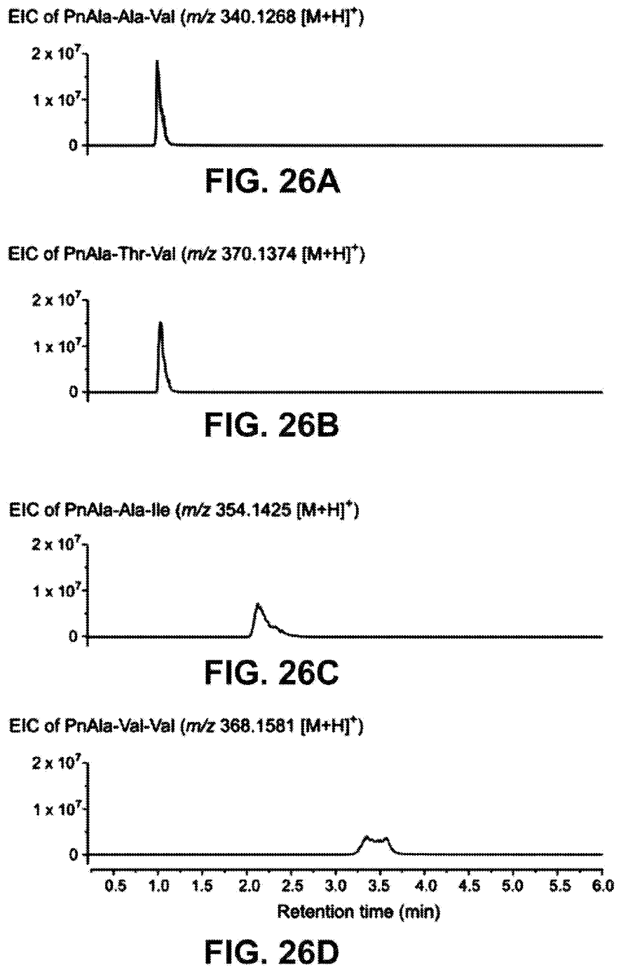

A total of 155 unique dipeptides were identified from the 146 positive reactions (Table 2). These included all 11 phosphonoalamide derivatives observed within crude cell extracts (FIG. 27). PnaC exhibited the broadest substrate specificity of any biochemically characterized L-amino acid dipeptide ligase (FIG. 4A, Table 3, FIG. 25A-FIG. 27) [B35-B45]. All 20 proteinogenic amino acids were accepted as nucleophiles, while all but arginine and cysteine were accepted as carboxylates. Grouping amino acids based on their side-chain properties (nonpolar, aromatic, polar uncharged, basic, and acidic) revealed patterns of substrate specificity. PnaC synthesized dipeptides by pairing combinations of amino acids from within and between each group (e.g. aromatic+aromatic, nonpolar+polar, and acidic+basic) with the sole exception of aromatic+acidic products. Notably, PnaC was not restricted by dipeptide size (forming Gly-Gly and Trp-Trp), polarity (forming Phe-Phe and Lys-Lys), or charge (forming doubly positive Lys-Lys, doubly negative Asp-Asp, and mixed charge Glu-Lys dipeptides).

TABLE 2
Summary of dipeptides.

| | |
|---|---|
| Ala-Ala, Gly-Ala, | Ala-Thr, Ser-Thr, |
| Pro-Ala | Gly-Thr, Pro-Thr |
| Ala-Val, Val-Val, | aThr-(4-¹³C-2,3-D₂-Thr) |
| Leu-Val, Ser-Val | |
| Thr-Val, aThr-Val, | Ala-aThr, Ser-aThr, |
| Asn-Val, Gln-Val | (4-¹³C-2,3-D₂-Thr)- |
| | aThr, aThr-aThr |
| Asp-Val, Glu-Val, | Glu-aThr, Gly-aThr |
| Gly-Val, Pro-Val | |
| Ala-Ile, Val-Ile, Ile-Ile, | Ala-Asn, Ser-Asn, |
| Leu-(¹⁵N-Ile) | Asp-Asn, Pro-Asn |
| Ser-Ile, Thr-Ile, | Ala-Gln, Ser-Gln, |
| aThr-Ile, Gln-Ile | Glu-Gln, Pro-Gln. |
| Asp-Ile, Gly-Ile, Pro-Ile | Ala-Arg, Val-Arg, |
| | Ile-Arg, Leu-Arg |
| Ala-Leu, Val-Leu, | Met-Arg, Phe-Arg, Tyr-Arg, |
| (¹⁵N-Ile)-Leu, Leu-Leu | Trp-Arg |
| Ser-Leu, Thr-Leu, | Ser-Arg, Thr-Arg, |
| aTHr-Leu, Gln-Leu | aThr-Arg, Gln-Arg |
| Gly-Leu, Pro-Leu | Asp-Arg, Gly-Arg, Pro-Arg |
| Ala-Met, Val-Met, | Ala-His, Val-His, |
| Leu-Met, Met-Met | Ile-His, Leu-His |
| Phe-Met, Tyr-Met, | Phe-His, Tyr-His, |
| Ser-Met, Thr-Met | Trp-His, Ser-His |
| aThr-Met, Gly-Met, | Thr-His, aThr-His, |
| Pro-Met | Asp-His, Glu-His |
| Ala-Phe, Val-Phe, Ile-Phe, Leu-Phe | Gly-His, Pro-His |
| Met-Phe, Phe-Phe, | Ala-Lys, Ile-Lys, |
| Tyr-Phe, Ser-Phe | Leu-Lys, Phe-Lys |
| Thr-Phe, aThr-Phe, | Tyr-Lys, Trp-Lys, |
| Gly-Phe, Pro-Phe | Ser-Lys, Thr-Lys |
| Ala-Tyr, Val-Tyr, | aThr-Lys, Asn-Lys, |
| Leu-Tyr | Lys-Lys, Asp-Lys |
| Met-Tyr, Phe-Tyr, Tyr-Tyr | Glu-Lys, Gly-Lys, Pro-Lys |
| Ser-Tyr, Gly-Tyr, Pro-Tyr | Asp-Asp |
| Ala-Trp, Val-Trp, Ile-Trp, Leu-Trp | Ala-Glu, Glu-Glu |
| Met-Trp, Phe-Trp, Tyr-Trp, Trp-Trp | Ala-Cys |
| Ser-Trp, Thr-Trp, Gly-Trp, Pro-Trp | Glu-Gly, Gly-Gly |
| Ala-Ser, Ser-Ser, Glu-Ser, Gly-Ser | aThr-Pro, His-Pro |

TABLE 3
Summary of dipeptides.

| | |
|---|---|
| X-Ala dipeptides | A) Ala-Ala m/z 161.0921, B) |
| synthesized in | Gly-Ala m/z 147.0764, C) Pro-Ala m/z |
| PnaC reactions | 187.1077. |
| X-Val dipeptides | A) Ala-Val m/z 189.1234, B) Val-Val |
| synthesized in | m/z 217.1547, C) Leu-Val m/z |
| PnaC reactions | 231.1703 , D) Ser-Val m/z 205.1183, E) |
| | Thr-Val m/z 219.1339, F) aThr- |
| | Val m/z 219.1339, G) Asn-Val m/z |
| | 232.1292, H) Gln-Val m/z 247.1448, |
| | I) Asp-Val m/z 233.1132, J) Glu-Val |
| | m/z 247.1289, K) Gly-Val m/z |
| | 175.1077, L) Pro-Val m/z 215.1390. |
| X-Ile dipeptides | A) Ala-Ile m/z 203.1390, B) Val-Ile |
| synthesized in | m/z 231.1703, C) Ile-Ile m/z |
| PnaC reactions | 245.1860, D) Leu-15N-Ile m/z 246.1830, |
| | E) Ser-Ile m/z 219.339, F) Thr- |
| | Ile m/z 233.1496, G) aThr-Ile m/z |
| | 233.1496, H) Gln-Ile m/z 260.1605, I) |
| | Asp-Ile m/z 247.1289, J) Gly-Ile m/z |
| | 189.1234, K) Pro-Ile m/z 229.1587. |
| X-Leu dipeptides | A) Ala-Leu m/z 203.1390, B) Val-Leu |
| synthesized in | m/z 231.1703, C) ¹⁵N-Ile-Leu m/z |
| PnaC reactions | 246.1830, D) Leu-Leu m/z 245.1860, |
| | E) Ser-Leu m/z 219.1339, F) Thr- |
| | Leu m/z 233.1496, G) aThr-Leu |
| | m/z 233.1496, H) Gln-Leu m/z |
| | 260.1605, I) Gly-Leu m/z 189.1234, |
| | J) Pro-Leu m/z 229.1547. |
| X-Met dipeptides | A) Ala-Met m/z 221.0954, B) Val-Met |
| synthesized in | m/z 249.1267, C) Leu-Met m/z |
| PnaC reactions | 263.1424, D) Met-Met m/z 281.0988, |
| | E) Phe-Met m/z 297.1267, F) Tyr- |
| | Met m/z 313.1217, G) Ser-Met m/z |
| | 237.0904, H) Thr-Met m/z 251.1060, |
| | I) aThr-Met m/z 251.1060, J) Gly-Met |
| | m/z 207.0798, K) Pro-Met m/z |
| | 247.1111. |
| X-Phe dipeptides | A) Ala-Phe m/z 237.1234, B) Val-Phe |
| synthesized in | m/z 265.1547, C) Ile-Phe m/z |
| PnaC reactions | 279.1703, D) Leu-Phe m/z 279.1703, |
| | E) Met-Phe m/z 297.1267, F) Phe- |
| | Phe m/z 313.1547, G) Tyr-Phe m/z |
| | 329.1496, H) Ser-Phe m/z 253.1183, |
| | I) Thr-Phe m/z 267.1339, J) aThr-Phe |
| | m/z 267.1339, K) Gly-Phe m/z |
| | 223.1077, L) Pro-Phe m/z 263.1390. |
| X-Tyr dipeptides | A) Ala-Tyr m/z 253.1183, B) Val-Tyr |
| synthesized in | m/z 281.1497, C) Leu-Tyr m/z |
| PnaC reactions | 295.1652 , D) Met-Tyr m/z 313.1217, |
| | E) Phe-Tyr m/z 329.1496, F) Tyr- |
| | Tyr m/z 345.1445, G) Ser-Tyr m/z |
| | 269.1132, H) Gly-Tyr m/z 239.1026, |
| | I) Pro-Tyr m/z 279.1339. |
| X-Trp dipeptides | A) Ala-Trp m/z 276.1343, B) Val-Trp |
| synthesized in | m/z 304.1656, C) Ile-Trp m/z |
| PnaC reactions | 318.1812 , D) Leu-Trp m/z 318.1812, |
| | E) Met-Trp m/z 336.1376, F) Phe- |
| | Trp m/z 352.1656, G) Tyr-Trp m/z |
| | 368.1605, H) Trp-Trp m/z 391.1765, |
| | I) Ser-Trp m/z 292.1292, J) Thr-Trp |
| | m/z 306.1448, K) Gly-Trp m/z |
| | 262.1186, L) Pro-Trp m/z 302.1499. |
| X-Ser dipeptides | A) Ala-Ser m/z 177.0870, B) |
| synthesized in | Ser-Ser m/z 193.0819, C) Glu-Ser m/z |
| PnaC reactions | 235.0925 , D) Gly-Ser m/z 163.0713. |
| X-Thr dipeptides | A) Ala-Thr m/z 191.1026, B) Ser-Thr |
| synthesized in | m/z 207.0976, C) aThr-(4-¹³C-2,3- |
| PnaC reactions | D2-Thr) m/z 224.1291, D) Gly-Thr |
| | m/z 177.0870, E) Pro-Thr m/z |
| | 217.1183. |
| X-aThr dipeptides | A) Ala-aThr m/z 191.1026, B) Ser-aThr |
| synthesized in | m/z 207.0976, C) (4-¹³C-2,3-D2- |
| PnaC reactions | Thr)-aThr m/z 224.1291, D) aThr-aThr |
| | m/z 224.1291, E) Glu-aThr m/z |
| | 249.1081, F) Gly-aThr m/z 177.0870. |
| X-Asn dipeptides | A) Ala-Asn m/z 204.0979, B) Ser-Asn |
| synthesized in | m/z 220.0928, C) Asp-Asn m/z |
| PnaC reactions | 248.0877, D) Pro-Asn m/z 230.1135. |
| X-Gln dipeptides | A) Ala-Gln m/z 218.1134, B) Ser-Gln |
| synthesized in | m/z 234.1085, C) Glu-Gln m/z |
| PnaC reactions | 276.1190, D) Pro-Gln m/z 244.1292. |
| X-Arg dipeptides | A) Ala-Arg m/z 246.1561, B) Val-Arg |
| synthesized in | m/z 274.1874, C) Ile-Arg m/z |
| PnaC reactions | 288.2030, D) Leu-Arg m/z 288.2030, |
| | E) Met-Arg m/z 306.1594, F) Phe- |
| | Arg m/z 322.1874, G) Tyr-Arg m/z |
| | 338.1823, H) Trp-Arg m/z 361.1983, |
| | I) Ser-Arg m/z 262.1510, J) Thr-Arg |
| | m/z 276.1666, K) aThr-Arg m/z |
| | 276.1666, L) Gln-Arg m/z 303.1775, |
| | A) Asp-Arg m/z 290.1459, B) Gly- |
| | Arg m/z 232.1404, C) Pro-Arg m/z 272.1717. |
| X-His dipeptides | A) Ala-His m/z 227.1139, B) Val-His |
| synthesized in | m/z 255.1452, C) Ile-His m/z |
| PnaC reactions | 269.1608 , D) Leu-His m/z 269.1608, |
| | E) Phe-His m/z 303.1452, F) Tyr- |
| | His m/z 319.1401, G) Trp-His m/z |
| | 342.1561, H) Ser-His m/z 243.1077, I) |
| | Thr-His m/z 257.1244, J) aThr-His m/z |
| | 257.1244, K) Asp-His m/z |
| | 271.1037, L) Glu-His m/z 284.1194, |
| | A) m/z Gly-His 213.0982, B) Pro- |
| | His m/z 253.1295. |
| X-Lys dipeptides | A) Ala-Lys m/z 218.1499, B) Ile-Lys |
| synthesized in | m/z 260.1969, C) Leu-Lys m/z |
| PnaC reactions | 260.1969, D) Phe-Lys m/z 294.1812, |
| | E) Tyr-Lys m/z 310.1761, F) Trp- |
| | Lys m/z 333.1921, G) Ser-Lys m/z |
| | 234.1448, H) Thr-Lys m/z 248.1605, |
| | I) aThr-Lys m/z 248.1605, J) |
| | Asn-Lys m/z 261.1557, K) Lys-Lys m/z |
| | 275.2078, L) Asp-Lys m/z 262.1393, A) |
| | Glu-Lys m/z 267.1554, B) Gly- |
| | Lys m/z 204.1343, C) Pro-Lys m/z 244.1656. |
| X-Asp dipeptides | A) Asp-Asp m/z 249.0717. |
| synthesized in | |
| PnaC reactions | |
| X-Glu dipeptides | A) Ala-Glu m/z 219.0976, B) |
| synthesized in | Glu-Glu m/z 277.1030. |
| PnaC reactions | |
| X-Cys dipeptides | A) Ala-Cys m/z 193.0641. |
| synthesized in | |
| PnaC reactions | |
| X-Gly dipeptides | A) Glu-Gly m/z 205.0819, B) |
| synthesized in | Gly-Gly m/z 133.0608. |
| PnaC reactions | |
| X-Pro dipeptides | A) aThr-Pro m/z 217.1183, B) |
| synthesized in | His-Pro m/z 253.1295. |
| PnaC reactions | |

This data suggest that properties of the activated carboxylate residue influences specificity of the selected nucleophilic amino acid. This is in agreement with the model for the ATP-Grasp ligase MurD, where UDP-N-acetylmuramoyl-L-Ala binding enhances affinity for D-Glu [B46]. Even small differences between carboxylates, such as the single stereocenter differentiating Thr and allo-Thr, resulted in altered product profiles. Notably, activation of Thr led to acceptance of Trp as a nucleophile, but not Thr or Pro. In contrast, activation of allo-Thr resulted in both Thr and Pro as nucleophiles, to the exclusion of Trp. These results suggest the likely sensitivity of PnaC to small conformational changes.

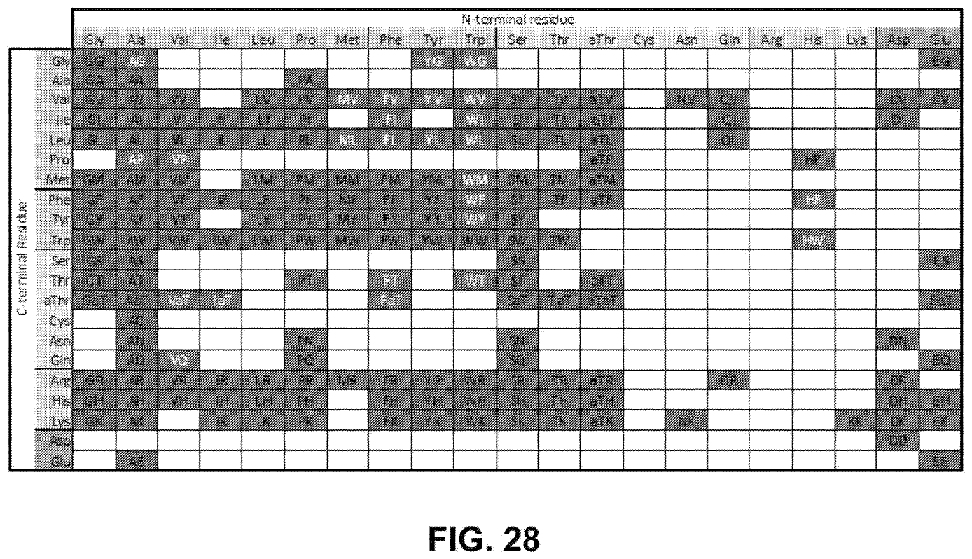

For PnaC, activation of a small nonpolar amino acids resulted in the greatest proportion of successful reactions (74 of 147; 54.1%). In comparison, activation of a basic amino acids resulted in the smallest proportion of reaction (2 of 63; 3.2%). Activation of aromatic (18 of 63; 28.6%), uncharged polar (42 of 126; 33.3%), and acidic (15 of 42; 35.7%) amino acids demonstrated intermediate success (FIG. 28). These preferences were also reflected in overall product yield. The most abundant dipeptides contained small nonpolar amino acids. Ala was clearly the most preferred substrate, yielding dipeptides with an average ion intensity nearly a magnitude larger than any other group (FIG. 4A).

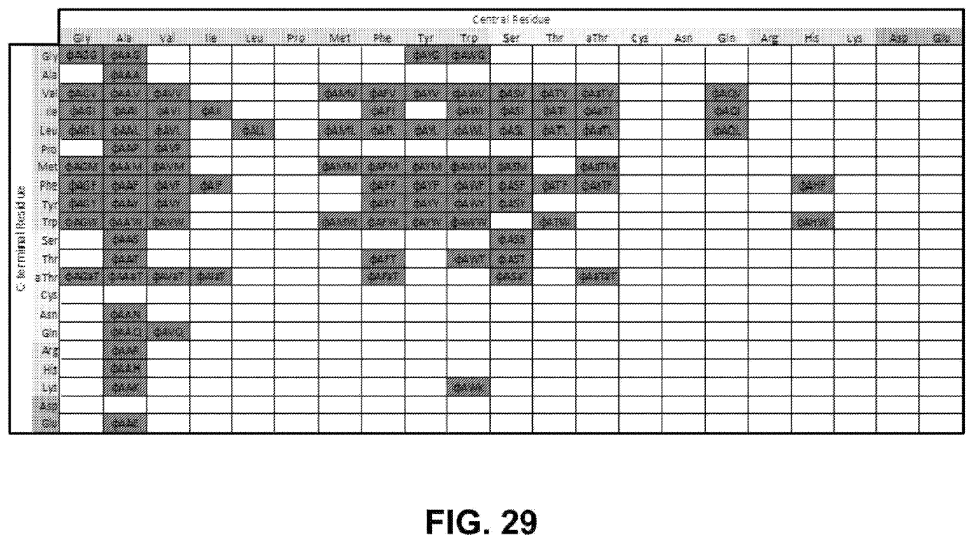

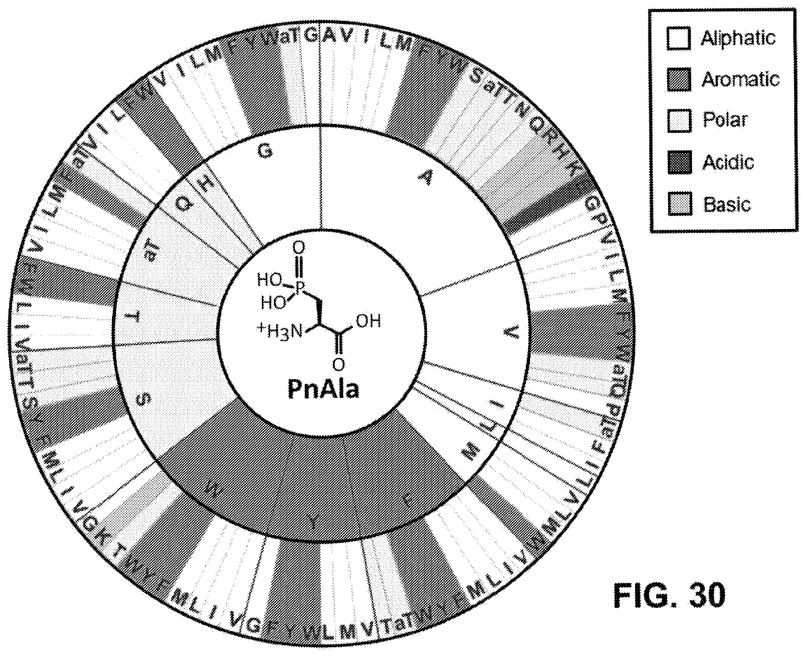

Having established the diversity of dipeptides produced by PnaC, the reaction specificity of PnaB was probed. PnaC was used to generate substrates for the ligation of PnAla by PnaB. Microscale reactions contained PnaB, PnaC, PnAla, and all pairs of amino acids shown to form dipeptides. LC-HRMS and fragmentation analysis was then used to identify and verify PnAla-containing tripeptides from these 80 reactions (FIG. 4B, Table 4, Table 5). Altogether, the combined enzyme system produced 97 distinct PnAla-tripeptides, 93 of which had not been previously isolated (FIG. 29 & FIG. 30).

TABLE 4
Summary of tripeptides

| | |
|---|---|
| PnAla-Ala-X tripeptides | PnAla-Ala-Ala m/z 312.0961, PnAla-Ala-Val |
| synthesized in | m/z 340.1274, PnAla-Ala-Ile m/z 354.1430, |
| PnaBC reactions | PnAla-Ala-Leu m/z 354.1430, PnAla-Ala-Met |
| | m/z 372.0994, PnAla-Ala-Phe m/z 388.1274, |
| | PnAla-Ala-Tyr m/z 404.1223, PnAla-Ala-Trp |
| | m/z 427.1383, PnAla-Ala-Ser m/z 328.0910, |
| | PnAla-Ala-Thr m/z 342.1061, PnAla-Ala- |
| | aThr m/z 342.1061, PnAla-Ala-Asn m/z |
| | 355.1019, PnAla-Ala-Gln m/z 369.1175, |
| | PnAla-Ala-Arg m/z 397.1601 , PnAla-Ala- |
| | His m/z 378.1179, PnAla-Ala-Lys m/z |
| | 369.1539, PnAla-Ala-Glu m/z 370.1010, |
| | PnAla-Ala-Gly m/z 298.0799, PnAla-Ala-Pro |
| | m/z 338.1112. |
| PnAla-Val-X tripeptides | PnAla-Val-Val m/z 368.1587, PnAla-Val-Ile |
| synthesized in | m/z 382.1738, PnAla-Val-Leu m/z 382.1738, |
| PnaBC reactions | PnAla-Val-Met m/z 400.1302, PnAla-Val-Phe |
| | m/z 416.1581, PnAla-Val-Tyr m/z 432.1530, |
| | PnAla-Val-Trp m/z 455.1690, PnAla-Val- |
| | aThr m/z 370.1374, PnAla-Val-Gln m/z |
| | 397.1483, PnAla-Val-Pro m/z 366.1425, |
| | PnAla-Ile-Ile m/z 396.1894, PnAla-Ile-Phe |
| | m/z 430.1738 , PnAla-Ile-aThr m/z 384.1530. |
| PnAla-Leu-X tripeptides | PnAla-Leu-Leu m/z 396.1894. |
| synthesized in | |
| PnaBC reactions | |
| PnAla-Met-X tripeptides | PnAla-Met-Val m/z 400.1302, PnAla-Met- |
| synthesized in | Leu m/z 414.1458, PnAla-Met-Met m/z |
| PnaBC reactions | 432.1028, PnAla-Met-Trp m/z 487.1411. |
| PnAla-Phe-X tripeptides | PnAla-Phe-Val m/z 416.1581, PnAla-Phe-Ile |
| synthesized in | m/z 430.1738, PnAla-Phe-Leu m/z 430.1738, |
| PnaBC reactions | PnAla-Phe-Met m/z 448.1302, PnAla-Phe- |
| | Phe m/z 464.1581, PnAla-Phe-Tyr m/z |
| | 480.1530, PnAla-Phe-Trp m/z 503.1690, |
| | PnAla-Phe-Thr m/z 418.1374, PnAla-Phe- |
| | aThr m/z 418.1374. |
| PnAla-Tyr-X tripeptides | PnAla-Tyr-Val m/z 432.1530, PnAla-Tyr- |
| synthesized in | Leu m/z 446.1687, PnAla-Tyr-Met m/z |
| PnaBC reactions | 464.1251, PnAla-Tyr-Phe m/z 480.1530, |
| | PnAla-Tyr-Tyr m/z 496.1479, PnAla-Tyr-Trp |
| | m/z 519.1639, PnAla-Tyr-Gly m/z 390.1061. |
| PnAla-Trp-X tripeptides | PnAla-Trp-Val m/z 455.1690, PnAla-Trp-Ile |
| synthesized in | m/z 469.1847, PnAla-Trp-Leu m/z 469.1847, |
| PnaBC reactions | PnAla-Trp-Met m/z 487.1411, PnAla-Trp-Phe |
| | m/z 503.1690, PnAla-Trp-Tyr m/z 519.1639, |
| | PnAla-Trp-Trp m/z 542.1799, PnAla-Trp-Thr |
| | m/z 457.1483, PnAla-Trp-Lys m/z 484.1956, |
| | PnAla-Trp-Gly m/z 413.1221. |
| PnAla-Ser-X tripeptides | PnAla-Ser-Val m/z 356.1223, PnAla-Ser-Ile |
| synthesized in | m/z 370.1374, PnAla-Ser-Leu m/z 370.1374, |
| PnaBC reactions | PnAla-Ser-Met m/z 388.0938, PnAla-Ser-Phe |
| | m/z 404.1217, PnAla-Ser-Tyr m/z 420.1166, |
| | PnAla-Ser-Ser m/z 344.0859, PnAla-Ser-Thr |
| | m/z 358.1010, PnAla-Ser-aThr m/z 358.1010. |
| PnAla-Thr-X tripeptides | PnAla-Thr-Val m/z 370.1374, PnAla-Thr-Ile |
| synthesized in | m/z 384.1530, PnAla-Thr-Leu m/z 384.1530, |
| PnaBC reactions | PnAla-Thr-Phe m/z 418.1374, PnAla-Thr-Trp |
| | m/z 457.1483. |
| PnAla-aThr-X tripeptides | PnAla-aThr-Val m/z 370.1374, PnAla-aThr- |
| synthesized in | Ile m/z 384.1530, PnAla-aThr-Leu m/z |
| PnaBC reactions | 384.1530, PnAla-aThr-Met m/z 402.1095, |
| | PnAla-aThr-Phe m/z 418.1374, PnAla-aThr- |
| | aThr m/z 372.1166. |
| PnAla-Gln-X tripeptides | PnAla-Gln-Val m/z 397.1483, PnAla-Gln-Ile |
| synthesized in | m/z 411.1639, PnAla-Gln-Leu m/z 411.1639. |
| PnaBC reactions | |
| PnAla-His-X tripeptides | PnAla-His-Phe m/z 454.1486, PnAla-His-Trp |
| synthesized in | m/z 493.1595. |
| PnaBC reactions | |
| PnAla-Gly-X tripeptides | PnAla-Gly-Val m/z 326.1117, PnAla-Gly-Ile |
| synthesized in | m/z 340.1268, PnAla-Gly-Leu m/z 340.1268, |
| PnaBC reactions | PnAla-Gly-Met m/z 358.0832, PnAla-Gly- |
| | Phe m/z 374.1112, PnAla-Gly-Tyr m/z |
| | 390.1061, PnAla-Gly-Trp m/z 413.1221, |
| | PnAla-Gly-aThr m/z 328.0904, PnAla-Gly- |
| | Gly m/z 284.0648. |

TABLE 5
Summary of Tripeptides

| | |
|---|---|
| PnAla-Ala-Ala, PnAla-Ala-Val, PnAla-Ala-Ile | PnAla-Ala-Pro |
| PnAla-Ala-Leu, PnAla-Ala-Met, PnAla-Ala-Phe | PnAla-Val-Pro |
| PnAla-Ala-Tyr, PnAla-Ala-Trp, PnAla-Ala-Ser | PnAla-Met-Trp |
| PnAla-Ala-Thr, PnAla-Ala-aThr, PnAla-Ala-Asn | PnAla-Trp-Gly |
| PnAla-Ala-Gln, PnAla-Ala-Arg, PnAla-Ala-His | PnAla-Ser-Val, |
| | PnAla-Ser-Ile |
| PnAla-Ala-Lys, PnAla-Ala-Glu, PnAla-Ala-Gly | PnAla-Ser-Leu, |
| | PnAla-Ser-Met |
| PnAla-Val-Val, PnAla-Val-Ile, PnAla-Val-Leu | PnAla-Ser-Phe, |
| | PnAla-Ser-Tyr |
| PnAla-Val-Met, PnAla-Val-Phe, PnAla-Val-Tyr | PnAla-Thr-Phe, |
| | PnAla-Thr-Trp |
| PnAla-Val-Trp, PnAla-Val-aThr, PnAla-Val-Gln | PnAla-Leu-Leu |
| PnAla-Ile-Ile, PnAla-Ile-Phe, PnAla-Ile-aTh | PnAla-Tyr-Gly |
| PnAla-Met-Val, PnAla-Met-Leu, PnAla-Met-Met | PnAla-His-Phe, |
| | PnAla-His-Trp |
| PnAla-Phe-Val, PnAla-Phe-Ile, PnAla-Phe-Leu. | |
| PnAla-Phe-Met, PnAla-Phe-Phe, PnAla-Phe-Tyr. | |
| PnAla-Phe-Trp, PnAla-Phe-Thr, PnAla-Phe-aThr. | |
| PnAla-Tyr-Val, PnAla-Tyr-Leu, PnAla-Tyr-Met | |
| PnAla-Tyr-Phe, PnAla-Tyr-Tyr, PnAla-Tyr-Trp | |
| PnAla-Trp-Val, PnAla-Trp-Ile, PnAla-Trp-Leu | |
| PnAla-Trp-Met, PnAla-Trp-Phe, PnAla-Trp-Tyr | |
| PnAla-Trp-Trp, PnAla-Trp-Thr, PnAla-Trp-Lys | |
| PnAla-Ser-Ser, PnAla-Ser-Thr, PnAla-Ser-aThr | |
| PnAla-Thr-Val, PnAla-Thr-Ile, PnAla-Thr-Leu | |
| PnAla-aThr-Val, PnAla-aThr-Ile, PnAla-aThr-Leu | |
| PnAla-aThr-Met, PnAla-aThr-Phe, PnAla-aThr-aThr | |
| PnAla-Gln-Val, PnAla-Gln-Ile, PnAla-Gln-Leu | |
| PnAla-Gly-Val, PnAla-Gly-Ile, PnAla-Gly-Leu | |
| PnAla-Gly-Met, PnAla-Gly-Phe, PnAla-Gly-Tyr | |
| PnAla-Gly-Trp, PnAla-Gly-aThr, PnAla-Gly-Gly | |

Nearly half of the dipeptides produced by PnaC were accepted by PnaB as nucleophiles to form PnAla tripeptides. PnaB preferred dipeptides with smaller N-terminal side chains, while largely avoiding those bearing charges. All alanyl dipeptides, with the exception of Ala-Cys and Ala-Asp, was ligated to PnAla. Tripeptides produced in the greatest abundance included PnAla conjugates of Ala-Ala, Ala-Val, Ala-Ile, Ala-Leu, and Ala-Met. Aversion for charged side chains was reflected by the limited number of PnAla tripeptides (7) that contained Arg, His, Lys, Asp, or Glu. (FIG. 4B & FIG. 30).

This data also suggested that PnaB modulates the dipeptide synthetase activity of PnaC. Nearly a quarter of the PnAla tripeptides produced from one-pot reactions contained dipeptides that were not observed in reactions with PnaC alone (FIG. 28). This trend was observed among tripeptides containing Ala, Val, Ile, Met, Phe, Tyr, Trp, and His in the central position. The differences were most pronounced in PnaBC reactions containing Met and Trp, and Ala and Pro. Surprisingly, PnaBC produced PnAla-Ala-Pro and PnAla-Val-Pro tripeptides even though only Pro-Ala and Pro-Val were detected in the PnaC-only reactions. Also unexpected was the production of PnAla-Trp-Met (major species) and PnAla-Met-Trp by the two-enzyme system. This contrasted with the PnaC-only reactions, where Met-Trp was detected as the sole product. A total of 26 “hidden” dipeptides were revealed within the tripeptide synthesis data, raising the total number of dipeptides produced by PnaC to 181 (FIG. 28).

In these cases, it was reasoned that PnaB acts as a coupling enzyme for PnaC. For a given pair of amino acids, PnaC may have inherent preference for which is the activated carboxylate species and which performs nucleophilic attack. This may manifest as different ratios of the dipeptides, including those at concentrations below the limits of detection in the assays. However, selectivity of PnaB for ligating PnAla with the minor products (e.g., Trp-Met) would in turn drive their synthesis by PnaC. While the specificity of production of dipeptides by LALs can be shifted by modulating host amino acid flux [B47], this is the first example of an oligopeptide ligase directing the activity of a preceding dipeptide ligase within the same biosynthetic pathway.

CONCLUSIONS. Using a combination of comparative genomics, heterologous expression, gene deletion, and biochemical reconstitution experiments, the complete biosynthetic pathway for the Streptomyces phosphonoalamides has been elucidated. Of the 11 contiguous genes conserved between Streptomyces strains, only 4 encoded enzymes were essential for biosynthesis. The pathway begins with the isomerization of PEP to PnPy by PepM (PnaD), coupled to the immediate transamination of PnPy to PnAla by PnaA. Concurrently, PnaC ligates two amino acids to generate a wide variety of dipeptides. The pathway then converges, as PnaB ligates the PnAla produced by PnaD to a dipeptide produced by PnaC, resulting in a diverse array of phosphonoalamides. It was noted that the two enzymatic steps from PEP to PnAla represent the shortest biosynthetic pathway towards a bioactive Pn compound.

While the transamination of PnPy by PnaA is a reversible reaction and its directionality is influenced by substrate concentration, kinetic analyses of PnaA's inherent catalytic properties and reported intracellular metabolite concentrations indicate that PnAla is likely to be efficiently produced in vivo. Aspartate, the preferred amino donor for PnAla formation, is the second most abundant amino acid in both E. coli and S. coelicolor [B48, B49]. In E. coli, the intracellular concentration of aspartate is 220 times greater than that of OAA, such that metabolic flux may further favor the transamination of PnPy to PnAla and concurrent conversion of aspartate to OAA [B50]. The ability to drive the PnaA reaction in reverse also offers a route for biocatalytic generation of PnPy, which is valuable as both a chemical synthon and substrate for biochemical studies of Pn metabolism.

PnaB and PnaC add to the growing number of ATP-grasp amino acid ligases involved in phosphonopeptide biosynthesis. Both exhibited extremely broad specificity, producing more than 10 PnAla-tripeptides within strains and 87 Pn-Ala tripeptides by direct enzymatic synthesis. Indeed, ATP-Grasp ligases may underly a strategy to produce multiple phosphonopeptides from one biosynthetic pathway. The rhizocticin, plumbemycin, and valinophos pathways encode ATP-grasp ligases and can produce multiple compounds all with the same Pn headgroup [B10, B39, B40, B51]. In contrast, phosphonopeptides biosynthesized using non-ribosomal peptide synthetase (phosphothricin tripeptide, phosalacine) [B5, B52] or the tRNA-dependent GCN5-related N-acetyltransferase family enzymes (argolaphos, dehydrophos, fosfazinomycin) [B11, B53-B55] are invariable in the amino acid composition of their products, suggesting these ligases are highly specific.

The ability to generate multiple phosphonopeptides may provide producing bacteria advantages against other competitor microorganisms sharing the same environmental niche. Phosphonopeptides are commonly utilize a “Trojan horse” mechanism to manifest their bioactivity and organismal specificity. The composition of amino acids attached to the Pn moiety mediates recognition and import by different oligopeptide transporters, after which hydrolysis by endogenous peptidases releases the active Pn moiety [B56-B58]. This is reflected in the rhizocticins and plumbemycins, both of which contain the threonine synthase inhibitor (Z)-L-2-amino-5-phosphono-3-pentenoic acid, but exhibit selectivity as antifungal or antibacterial agents based upon the composition of ligated amino acids. Within the phosphonoalamides, phosphonoalamide A (PnAla-Ala-Val, from Streptomyces) and phosphonoalamide F (Ala-Ala-PnAla, from Bacillus) display differing spectrums of antimicrobial activity [B8, B9]. In this manner, production of a diverse array of tripeptides may allow for an enhanced spectrum of antimicrobial activity as opposed to individual compounds. Complementarily, the versatility of PnaB and PnaC could also serve as a self-resistance mechanism. The incorporation of tabtoxinine-β-lactam into tabtoxin by TblF has been proposed as a mode of self-protection [B59], and incorporation of PnAla into tripeptides would similarly provide a pathway for sequestering the toxic free Pn.

It has not escaped attention that PnaC serves as an ideal starting point for the rational engineering of dipeptide ligases. PnaC has the broadest specificity of all biochemically characterized dipeptide ligases, accepting all proteinogenic amino acids as nucleophiles and all but arginine and cysteine as carboxylates. PnaC produced numerous important dipeptides, including Ala-Gln, (used in patient infusion for nutrients), Leu-Ile (antidepressant effect), and Leu-Ser (salty taste enhancer) [B37]. It is likely that only small changes would be required for PnaC to accept Arg and Cys as carboxylates, as single mutations have been shown to significantly alter LAL specificity [B60]. A greater understanding of the molecular determinants of ligase substrate specificity will enable improved prediction of peptide natural products encoded by biosynthetic gene clusters and empower their application as biocatalysts.

The biosynthetic pathway for the Streptomyces phosphonoalamides establishes transamination of PnPy as a branch of phosphonate and phosphinate natural product metabolism, which is broadly distributed among taxonomically diverse organisms and environments. Peptide ligation reactions were employed to produce an extensive series of PnAla-containing phosphonopeptides, emphasizing the diversity of products resulting from a single biosynthetic gene cluster. More broadly, these findings highlight the wealth of PnAla-containing natural products which await discovery.

### Materials and Methods

Chemicals. General chemical reagents were purchased from Sigma-Aldrich, Fisher Scientific, VWR, or Santa Cruz Biotechnology. Stable isotopes were purchased from Cambridge Isotope Laboratories.

Strains, Media, General Culture Conditions. The strains and plasmids used in this study are listed in Supplemental Table 6 and Table 7. Escherichia coli strains were routinely grown on LB broth or agar at 37° C. Streptomyces strains were grown at 30° C. The following additives and antibiotics were included for plasmid maintenance and selection as appropriate: 20 μg mL⁻¹ 2,6-diaminopimelic acid (DAP), 25 μg mL⁻¹ kanamycin (Km), 100 μg mL⁻¹ ampicillin (Amp), 15 μg mL⁻¹ chloramphenicol (Clm), 25 μg mL⁻¹ apramycin (Apr). All components were dissolved in deionized water (dI). For plates, 16 g agar was added per liter of media. All media formulations are given per liter.

ATCC 172: 20 g Soluble starch (potato), 10 g glucose, 5 g yeast extract, 5 g N-Z Amine Type A, 1 g CaCO₃. Adjusted to pH 7.3 prior to autoclaving.

Balch's vitamins solution [S1]: 5 mg p-Aminobenzoic acid, 2 mg folic acid, 2 mg biotin, 5 mg nicotinic acid, 5 mg calcium pantothenate, 5 mg riboflavin, 5 mg thiamine HCl, 10 mg pyridoxine HCl (B6), 100 μg cyanocobalamin (B12), 5 mg thioctic acid (lipoic acid). Adjusted pH to 7.0 with 1M NaOH. Filter sterilized.

GUBC: 10 g Sucrose, 5 g beef extract, 5 g Casamino acids, 10 mL 50% glycerol solution (w/v), 5 mL 1 M Na₂HPO₄—KH₂PO₄ buffer (pH 7.3), 2 mL Hunter's Concentrated Base. 10 mL of filter-sterilized Balch's vitamins added after autoclaving.

Hunter's Concentrated Base [S2]: 20 g Nitrilotriacetic acid, 14 g KOH, 59.3 g MgSO₄-7H₂O, 6.67 g CaCl₂-2H₂O, 18.5 mg (NH₄)₆Mo₇O₂₄-4H₂O, 0.198 g FeSO₄-7H₂O, 100 mL Hunter's Metals 44. Adjusted pH to 6.8 with 10M KOH.

Hunter's Metals 44: 2.5 g EDTA (free acid), 10.95 g ZnSO₄-7H₂O, 5 g FeSO₄—H₂O, 1.54 g MnSO₄—H₂O, 0.392 g CuSO₄-5H₂O, 0.25 g Co(NO₃)₂-6H₂O, 0.177 g Na₂B₄O₇-10H₂O. Acidified with 5 drops of sulfuric acid.

LB: 10 g Tryptone, 5 g yeast extract, 5 g NaCl.

ISP2: 10 g Malt extract, 4 g yeast extract, 4 g glucose.

ISP4: 10 g Soluble starch (potato), 2 g (NH₄)₂SO₄, 2 g CaCO₃, 1 g K₂HPO₄, 1 g MgSO₄-7H₂O, 1 mg FeSO₄-7H₂O, 1 mg ZnSO₄-7H₂O, 1 mg MnCl₂-2H₂O.

M9: 12.8 g Na₂HPO₄-7H₂O, 3 g KH₂PO₄, 1 g NH₄Cl, 0.5 g NaCl. MgSO₄ (2 mL) and CaCl₂ (100 μL) were added from 1 M filter-sterilized stock solutions after autoclaving.

MS1: 10 g Mannitol, 10 g roasted soy flour.

R2AS: 0.5 g Yeast extract, 0.5 g peptone, 0.5 g Casamino acids, 0.5 g glucose, 0.5 g soluble starch (potato), 0.3 g sodium pyruvate, 0.3 g K₂HPO₄, 0.05 g of MgSO₄-7H₂O. 10 mL sterile Balch's Vitamins and 20 mL sterile 1M sodium succinate were added after autoclaving.

TABLE 6
List of strains used in this study

| | | Source or |
|---|---|---|
| Name | Description | Reference |
| E. coli DH5a λ-pir | General cloning host | — |
| E. coli BL21(DE3) | Source of ser A gene | Novagen |
| E. coli Rosetta(DE3) | Protein overproduction host | Novagen |
| pLysSRARE | | |
| E. coli WM4489 | Cloning host for fosmids; | [S15] |
| | rhamnose inducible copy | |
| | control | |
| E. coli WM6029 | Host for conjugation, | [S16] |
| | acidauxotroph for 2,6- | |
| | diaminopimelic | |
| S. lividans 66 | Heterologous host | USDA NRRL |
| S. coelicolor A3(2) | Source of mdh gene | USDA NRRL |
| Streptomyces sp. | Phosphonoalamide A-D | USDA NRRL |
| NRRL B-2790 | producing strain | |
| Streptomyces sp. | Phosphonoalamide A-D | USDA NRRL |
| NRRL S-515 | producing strain | |
| Streptomyces sp. | Phosphonoalamide A-D | USDA NRRL |
| NRRL S-448 | producing strain | |
| S. lividans KSJ4338 | Derivative of S. lividans 66 | This study |
| | φC31 attB::pKSJ553 | |
| S. lividans KSJ4337 | Derivative of S. lividans 66 | This study |
| | φC31 attB::pKSJ554 | |
| S. lividans KSJ4362 | Derivative of S. lividans 66 | This study |
| | φC31 attB::pKSJ588 | |
| S. lividans KSJ4624 | Derivative of S. lividans 66 | This study |
| | φC31 attB::pKSJ595 | |
| S. lividans KSJ4625 | Derivative of S. lividans 66 | This study |
| | φC31 attB::pKSJ596 | |

TABLE 7
List of plasmids used in this study

| Plasmid | Description | Source |
|---|---|---|
| pAE4 | Encodes oriT, ΦC31 integrase, ΦC31 | [S15] |
| | attP for conjugation and site- | |
| | specific recombination into in most | |
| | streptomycete chromosomes; | |
| | Apr^{R} | |
| pAE5 | Source of aph(3′)-II cassette for gene | [S15] |
| | deletions; Km^{R} | |
| pJK50 | Dual-cos fosmid containing λattB site | [S15] |
| | allowing integration with pAE4; Clm^{R} | |
| pET28B | T7 based expression vector, | Novagen |
| | N/C-terminal His₆, Km^{R} | |
| 2-ST | pET His6 Sumo TEV LIC cloning | Addgene |
| | vector (2S-T), Amp^{R} | |
| pKSJ409 | Derivative of pET28B, overexpression | This study |
| | and purification of His₆-PnaD; Km^{R} | |
| pKSJ441 | Derivative of pET28B, overexpression | This study |
| | and purification of malate | |
| | dehydrogenase (His₆-MDH); Km^{R} | |
| pKSJ521 | Derivative of pET28B, overexpression | This study |
| | and purification of His₆-PnaB; Km^{R} | |
| pKSJ522 | Derivative of pET28B, overexpression | This study |
| | and purification of His₆-PnaC, Km^{R} | |
| pKSJ546 | pJK50 derivative containing 5 | This study |
| | upstream genes, orf1-5, and pnaA-D | |
| | from Streptomyces sp. S-515; Clm^{R} | |
| pKSJ549 | pJK50 derivative containing orf1-5, | This study |
| | pnaA-D, pnaT, orf11, and 2 | |
| | downstream genes from | |
| | Streptomyces sp. S-515; Clm^{R} | |
| pKSJ553 | Integration of pAE4 to the λattB | This study |
| | site of pKSJ546; Apr^{R}, Clm^{R} | |
| pKSJ554 | Integration of pAE4 to the λattB | This study |
| | site of pKSJ549; Apr^{R}, Clm^{R} | |
| pKSJ588 | Derivative of pKSJ554; cloned | This study |
| | S-515 sequence upstream of orf2 | |
| | replaced by the aph(3′)-II cassette | |
| | of pAE5; Km^{R}, Apr^{R}, Clm^{R} | |
| pKSJ595 | Derivative of pKSJ553; cloned | This study |
| | S-515 sequence upstream of pnaA | |
| | replaced by the aph(3′)-II cassette | |
| | of pAE5; Km^{R}, Apr^{R}, Clm^{R} | |
| pKSJ596 | Derivative of pKSJ554; cloned | This study |
| | S-515 sequence upstream of pnaA | |
| | replaced by the aph(3′)-II cassette | |
| | of pAE5; Km^{R}, Apr^{R}, Clm^{R} | |
| pKSJ599 | Derivative of 2-ST, overexpression | This study |
| | and purification of His₆-SUMO- | |
| | PnaA; Amp^{R} | |
| pKSJ635 | Derivative of pET28B, overexpression | This study |
| | and purification of His₆-SerA; Km^{R} | |

Km^{R}: Kanamycin resistant;
Amp^{R}: Ampicillin resistant;
Clm^{R}: Chloramphenicol resistant;
Apr^{R}: Apramycin resistant

Molecular Biology. DNA manipulations were performed according to standard methods [53] and manufacturer protocols. Lysozyme, achromapeptidase, and RNase A were from Sigma-Aldrich, restriction endonucleases and recombinant shrimp alkaline phosphatase (SAP) were from New England Biolabs (Beverly, MA). Plasmids were routinely purified from using Zymopure miniprep or midiprep kits (Zymo Research, Irvine, CA). Genomic DNA was isolated from Streptomycetes as previously described [54].

PCR reactions to generate DNA fragments for cloning were performed using Phusion or Q5 DNA polymerase, whereas PCR reactions for verification of strain constructions and fosmid library screening used OneTaq DNA polymerase (New England Biolabs). DNA fragments for cloning were purified from agarose gel slices using a Zymoclean DNA extraction kit (Zymo Research). The HiFi DNA assembly mix (New England Biolabs) was used for Gibson assembly except only 250 of each component was used per reaction. Oligonucleotides (Life Technologies, Carlsbad, CA) are listed in Table 8. Sanger sequencing was performed at the Ohio State University Comprehensive Cancer Center Genomics Shared Resource facility.

TABLE 8
Primers used in this study

| Primer | Sequence (5′ to 3′) | Purpose |
|---|---|---|
| S515-pepM- | GATGAACAGCTTCGTCGCCAC | Fosmid library screening |
| screen-F | (SEQ ID NO: 1) | |
| S515-pepM- | CCCTGGTTGGCGTAGATGA | Fosmid library screening |
| screen-R | (SEQ ID NO: 2) | |
| orf1-left- | TccgccaccgcacggggcggcgcgggcggtggggcctcaCC | PCR amplification of aph-II |
| Redswap-F | TAGGtcgaggttgacatcttttgc (SEQ ID NO: 3) | cassette to replace genes |
| orf1-left- | TacattatacgaagttatcaagaagcttgagctcggatcCCTAG | upstream orf1 by λ-Red |
| Redswap-R | Gtcagaagaactcgtcaagaagg (SEQ ID NO: 4) | recombination |
| pnaA-left- | TggtgagggtggtgaggacggaacgggtacggcgggccgCC | PCR amplification of aph-II |
| Redswap-R | TAGGtcagaagaactcgtcaagaagg | cassette to replace genes |
| | (SEQ ID NO: 5) | upstream pnaA by λ-Red |
| | | recombination (orf1-left- |
| | | Redswap-F was used as the |
| | | forward primer) |
| pET28b-NdeI- | CATATGGCTGCCGCGCGGCACCAGGC | Production of linearized |
| R | (SEQ ID NO: 6) | pET28B with N-His₆ for |
| pET28b-XhoI- | CTCGAGCACCACCACCACCACCACTG | Gibson assembly |
| F | (SEQ ID NO: 7) | |
| 2ST-F | TGCATTGGATTGGAAGTACAG | Production of linearized 2- |
| | (SEQ ID NO: 8) | ST with N-His₆-SUMO for |
| 2ST-R | ATTGGAAGTGGATAACGGATC | Gibson assembly |
| | (SEQ ID NO: 9) | |
| pET28b-NHis- | TGCCGCGCGGCAGCCATATGtcaggtgacctccg | PCR amplification of pnaB |
| PnaB-F | gccgctc (SEQ ID NO: 10) | for Gibson assembly |
| pET28b-NHis- | TGGTGGTGGTGGTGCTCGAGtcatccegtegccc | cloning into pET28B |
| PnaB-R | cgaagtcctc (SEQ ID NO: 11) | |
| pET28b-NHis- | TGCCGCGCGGCAGCCATATGacccgtcacatcgc | PCR amplification of pnaC |
| PnaC-F | cttcctg (SEQ ID NO:12) | for Gibson assembly |
| pET28b-NHis- | TGGTGGTGGTGGTGCTCGAGtcagtgcgagtcgg | cloning into pET28B |
| PnaC-R | cggtccggac (SEQ ID NO: 13 | |
| 2ST-PnaA-F | TTATCCACTTCCAATtcacctgacategtccatcac | PCR amplification of pnaA |
| | (SEQ ID NO: 14) | for Gibson assembly |
| 2ST-PnaA-R | TTCCAATCCAATGCAatgcccgaccagcaggtcgac | cloning into 2-ST |
| | (SEQ ID NO: 15) | |
| S515-pepM-F | GCCTGGTGCCGCGCGGCAGCCATATGttcaa | PCR amplification of pnaD |
| | gcccgtttogtac (SEQ ID NO: 16) | for Gibson assembly |
| S515-pepM-R | AGTGGTGGTGGTGGTGGTGCTCGAGtcacgc | cloning into pET28B |
| | ggactcgagctcctg | |
| | (SEQ ID NO: 17) | |
| pET28B-NHis- | TGCCGCGCGGCAGCCATATGactegcactcccgt | PCR amplification of mdh |
| SCO4927-Fd | gaacgt (SEQ ID NO: 18) | for Gibson assembly |
| pET28B-NHis- | TGGTGGTGGTGGTGCTCGAGtcagatgaggccga | cloning into pET28B |
| SCO4927-Rv | gaccgc (SEQ ID NO: 19) | |
| pET28B-NHis- | TGCCGCGCGGCAGCCATATGgcaaaggtatcgct | PCR amplification of serA |
| SerA-F | ggag (SEQ ID NO: 20) | for Gibson assembly |
| pET28B-NHis- | TGGTGGTGGTGGTGCTCGAGttagtacagcagac | cloning in pET28B |
| SerA-R | gggcgcg (SEQ ID NO: 21) | |

Primers used in Gibson assembly. Lowercase represents oligonucleotides bound to the gene, and uppercase represents the oligonucleotides homologous to the vector.
Primers used in λ-Red recombination. Oligonucleotides on the 5′ side are homologous to upstream or downstream sequences of the target gene. Oligonucleotides on the 3' side bind to aph-II cassette from pAE5.

Genome sequencing, assembly, and annotation. Genomic DNA isolated from Streptomyces sp. strains NRRL B-2790 and S-448 was sequenced at SeqCenter (Pittsburgh, PA) using Oxford Nanopore and Illumina technologies. Genomes were assembled with Unicycler [S5], annotated using PGAP [S6], and deposited in NCBI within Bioproject PRJNA1021995. The phosphonoalamide biosynthetic gene cluster from Streptomyces sp. NRRL S-515 was deposited in NCBI under accession PP239091.

Bioinformatic analyses. Gene neighborhoods were annotated by NCBI Blast, Pfam, and CDD [S7-S9]. Synteny were analyzed based on the gene cluster similarity comparisons performed using Easyfig [S10].

Construction and screening of a Streptomyces sp. S-515 fosmid library. High-molecular weight genomic DNA was isolated from Streptomyces sp. S-515 and used to construct a fosmid library as previously described [S4]. The library was screened for pepM using primers S515-pepM-screen-F and S515-pepM-screen-R. Two fosmids, pKSJ546 and pKSJ549, were selected for further analysis. These were recombined in-vitro with pAE4 using BP Clonase II (Invitrogen, Carlsbad, CA) to yield pKSJ553 and pKSJ554.

Gene-deletions. Gene deletions were constructed using the k Red recombination method of Datsenko and Wanner as previously described [S4, S11]. Briefly, PCR primers were designed to amplify an included sequence homologous to the upstream and downstream of each target (Table 8). All primers were designed to amplify the kanamycin resistance cassette from plasmid pAE5 [S12]. This cassette includes a synthetic promoter to ensure expression of downstream genes. Resulting fosmids (pKSJ588, 595, 596) were verified with diagnostic restriction digestion analysis and Sanger sequencing using primers aph-seq-up and aph-seq-down to validate constructs. These primers bind within the Km^{R} cassette and produce sequencing reads that span outwards across the designed junctions.

Heterologous expression. Plasmid constructs were introduced for integration at the phiC31 attB locus of S. lividans 66 by conjugation from E. coli. Plasmids pAE4, pKSJ553, 554, 588, 595, and 596 were transformed into electrocompetent WM6029. Strains were grown in LB-Apr-DAP to OD₆₀₀ of 0.5-0.6 and 1 mL harvested by centrifugation, washed and re-suspended in 500 μL LB. Spore stocks of S. lividans 66 (50 μL per conjugation, titer 1×10⁹ CFU mL⁻¹) were thawed on ice, centrifuged, washed and re-suspended in 500 μL LB, and germinated at 50° C. for 10 min. Germinated spores and washed E. coli cells were combined, centrifuged at 6,000 rpm for 5 min. The cell pellet was re-suspended in 100 μL M9 media and spotted on MS10 plates in 5 l aliquots. Plates were dried in a biosafety cabinet, incubated at 30° C. for 16 hours, and flooded with 500 l of apramycin (2 mg ml⁻¹ stock). After 5 days, three exconjugants from each mating were struck for isolation on MS-Apr and purified by three successive passages from fully sporulated cultures. Genomic DNA was isolated from each strain and used for diagnostic PCR reactions. One primer bound to an internal region of the kanamycin resistance cassette, and the second primer bound upstream or downstream region of the disrupted gene. (Table 8).

Production of phosphonate natural products from native, heterologous expression, and deletion strains. Strains were cultivated in 20×150 mm test tubes containing 5 mL of ATCC 172 medium on an orbital shaker (200 rpm) at 30° C. for 4-5 days. Cultures of Streptomyces sp. S-515 and B-2790 were inoculated onto ISP2, ISP4, GUBC, and R2AS agar (200 L per plate). Cultures of S. lividans heterologous expression and deletion strains were inoculated onto GUBC and R2AS. After 10 days plates were frozen at −20° C. and thawed to recover 25 mL of liquid extracts from individual cultures (3 plates each). Extracts were then lyophilized. Dried samples were reconstituted in 1 mL dI H₂O and analyzed by mass spectrometry and NMR spectroscopy as described below.

NMR spectroscopy. NMR spectroscopy was performed at the OSU Campus Chemical Instrument Center. All NMR spectra were recorded at 25° C. on a Bruker Avance III HD Ascend 600 MHz spectrometer (600 MHz for ¹H, 150 MHz for ¹³C and 243 MHz for ³¹P) equipped with a Bruker 5 mm Smart Broadband Observe solution probe (BBFO), a Bruker Avance Neo 400 MHz spectrometer (400 MHz for ¹H, 100 MHz for ¹³C and 162 MHz for ³¹P) equipped with a 5 mm Prodigy Cryoprobe, or a Bruker Avance III HD Ascend 700 MHz spectrometer (700 MHz for ¹H, 176 MHz for ¹³C and 283 MHz for ³¹P) equipped with a 5 mm Triple-resonance Observe (TXO) cryoprobe. Proton and carbon chemical shifts are reported in 6 values relative to an external standard of 0.1% tetramethylsilane in D₂O. Phosphorus chemical shifts are reported in 6 values relative to an external standard of 85% phosphoric acid. ¹H-³¹P gHMBC (gradient Heteronuclear Multiple-Bond Correlation) spectra were collected after optimization of long-range proton-phosphorus coupling at 18 Hz. Spectra were processed in MestReNova 12 software.

Mass spectrometry. Weak-anion exchange was routinely used as a preparative step to enrich phosphonic acids from extracts and improve signal to noise with Chelex-100-Fe resin was previously described [S4].

Mass spectrometry analyses were performed on an Agilent 6540B Q-ToF system equipped with a 1260 Infinity II HPLC system as previously described [S4].

Samples were also analyzed on a Thermo Q-Exactive Orbitrap with a Vanquish-H UHPLC system. The data were acquired under high resolution mode (RP=70,000) with an AGC target of 1E6 and a maximum IT of 200 ms. For positive mode analyses, samples were diluted to a total volume of 100 uL with 85% MeCN and 0.1% formic acid, 5 μL of which was injected onto a Waters XBridge Amide (2.1×150 mm) HPLC column. The buffer used for UHPLC was H₂O with 0.1% formic acid (solvent A) and MeCN with 0.1% formic acid (solvent B). The flow rate was set at 0.35 mL/min. The elution gradient started at 85% solvent B for 2 minutes followed by a linear gradient to 40% solvent B over 4 min, a maintenance at 40% solvent B over 3 minutes, a return to 85% solvent B over 6 seconds, and re-equilibrated for 5.4 min before the next injection. For MS/MS, the same settings, gradient, and column were used. Target ion(s) were added to the inclusion list with a starting collision energy of 10 eV for dipeptides and 15 eV dipeptides, adjusted in 5 eV increments in subsequent runs if needed.

Expression and Purification of His6-SUMO-PnaA. The pnaA gene was amplified by PCR from the genomic DNA of S-515 using primers listed in Table 8. The product was gel purified and cloned into linearized 2-ST by Gibson assembly to yield pKSJ599, which encodes His₆-SUMO-PnaA. The plasmid was transformed into E. coli Rosetta (DE3) pLysSRARE. The strain was grown in 8 L LB-Amp-Clm at 37° C., 220 rpm to OD₆₀₀ 0.4 and cold shocked on ice for 10 min. Protein production was induced by the addition of IPTG to 0.2 mM and the culture returned to 16° C., 220 rpm for 16 hours. The culture was harvested by centrifugation, and the cell pellet was re-suspended in 60 mL lysis buffer (50 mM HEPES pH 7.5, 250 mM NaCl, 10 mM imidazole) containing 10 mg lysozyme and 100 U DNase. The suspension was gently mixed at room temperature for 20 min. Cells were lysed by sonication and centrifuged at 12,000 rpm for 30 min at 4° C.

Clarified cell lysates was combined with 5 mL HisTrap FF column at 2.5 mL min⁻¹ using an Akta Go FLPLC system. His₆-SUMO-PnaA was purified using buffer A (50 mM HEPES pH 7.5, 250 mM NaCl, 10% glycerol), and buffer B (50 mM HEPES pH 7.5, 250 mM NaCl, 10% glycerol, 500 mM imidazole) by the following program: 2 column volumes (CV) 6% buffer B; 1 CV 6-10% buffer B; 2 CV 10% buffer B; 4 CV 10-50% buffer B; 2 CV 50% buffer B; 10 CV 0% buffer B. Fractions containing the target protein (analyzed by Bradford and SDS-PAGE) were concentrated to 2.5 mL by 10 kDa Amicon Ultra-15 Millipore centrifugal filter and then desalted by PD-10 column with storage buffer (50 mM HEPES pH 7.5, 250 mM NaCl, 10% glycerol). Yield: 1.84 mg L⁻¹ of culture.

Expression and purification of His₆-PnaB. The pnaB gene was amplified by PCR from the genomic DNA of S-515 using primers listed in Table 8. The product was gel purified and cloned into linearized pET28B by Gibson assembly to yield pKSJ521, which encodes His₆-PnaB. The plasmid was transformed into E. coli Rosetta (DE3) pLysSRARE. The overproduction of His₆-PnaB was the same as His₆-SUMO-PnaA except 1 L LB-Km-Clm media was used. The culture was harvested by centrifugation, and the cell pellet was re-suspended in 20 mL lysis buffer (50 mM HEPES pH 7.5, 250 mM NaCl, 10 mM imidazole) containing 10 mg lysozyme and 100 U DNase. The suspension was gently mixed at room temperature for 20 min. Cells were lysed by sonication and centrifuged at 12,000 rpm for 30 min at 4° C.

Clarified cell lysates was combined with 5 mL HisPur™ Ni-NTA affinity resin (Thermo scientific) in a column and gently nutated at 4° C. for 30 min. The resin was washed with 100 mL wash buffer (50 mM HEPES pH 7.5, 250 mM NaCl, 30 mM imidazole, 10% glycerol), and elute with 20 mL elution buffer A (50 mM HEPES pH 7.5, 250 mM NaCl, 50 mM imidazole, 10% glycerol), 20 mL elution buffer B (50 mM HEPES pH 7.5, 250 mM NaCl, 100 mM imidazole, 10% glycerol), and 20 mL elution buffer C (50 mM HEPES pH 7.5, 250 mM NaCl, 250 mM imidazole, 10% glycerol). Fractions containing the target protein (analyzed by Bradford and SDS-PAGE) were concentrated to 2.5 mL by 10 kDa Amicon Ultra-15 Millipore centrifugal filter and then desalted by PD-10 column with storage buffer (50 mM HEPES pH 7.5, 250 mM NaCl, 10% glycerol). Yield: 19 mg L⁻¹ of culture.

Expression and purification of His6-PnaC. The pnaC gene was amplified by PCR from the genomic DNA of S-515 using primers listed in Table 8. The product was gel purified and cloned into linearized pET28B by Gibson assembly to yield pKSJ522, which encodes His₆-PnaC. The plasmid was transformed into E. coli Rosetta (DE3) pLysSRARE. The overproduction and purification procedures for His₆-PnaB were the same as His₆-PnaC. Yield: 14 mg L⁻¹ of culture.

Expression and purification of His6-PnaD. The pnaD gene was amplified by PCR from the genomic DNA of S-515 using primers listed in Table 8. The product was gel purified and cloned into linearized pET28B by Gibson assembly to yield pKSJ409, which encodes His₆-PnaD. The plasmid was transformed into E. coli Rosetta (DE3) pLysSRARE. The overproduction and purification procedures for His₆-PnaB were the same as His₆-PnaC. Yield: 26.1 mg L⁻¹ of culture.

Expression and purification of His6-VlpB. Recombinant phosphonopyruvate reductase (VlpB) was purified from expressed and purified from E. coli as previously described [S13].

Expression and purification of His6-MDH. The malate dehydrogenase gene (locus tag SC04927) was amplified by PCR from the genomic DNA of S. coelicolor A3(2) using primers listed in Table 8. The product was gel purified and cloned into linearized pET28B by Gibson assembly to yield pKSJ441, which encodes His₆-MDH. The overproduction and purification procedures for His₆-MDH were the same as His₆-PnaC. Yield: 25 mg L⁻¹ of culture.

Expression and purification of His6-SerA. The serA gene was amplified by PCR from the genomic DNA of E. coli BL21 (DE3) using primers listed in Table 8. The product was gel purified and cloned into linearized pET28B by Gibson assembly to yield pKSJ635, which encodes His₆-MDH. The overproduction and purification procedures for His₆-MDH were the same as His₆-PnaC. Yield: 12 mg L⁻¹ of culture.

Biochemical assays of His6-SUMO-PnaA with His6-PnaD. Typical reaction mixtures (300 μL) contained 20 μM His₆-PnaD, 20 μM His₆-SUMO-PnaA, 1.5 mM PEP, 3 mM L-Asp, 100 μM pyridoxal 5′-phosphate (PLP), and 2 mM MgCl₂ in 50 mM HEPES, 150 mM NaCl, pH 7.5. Reactions were incubated at 30° C. for 2 hours, heat inactivated at 65° C. for 10 minutes, and then analyzed by ³¹P NMR and LC-MS as described above.

Reactions in timecourse experiments (150 L) contained 10 μM His₆-PnaD, 10 μM His₆-SUMO-PnaA, 1.5 mM PEP, 10 mM L-Asp, 100 μM pyridoxal 5′-phosphate (PLP), and 2 mM MgCl₂ in 50 mM HEPES, 150 mM NaCl, pH 7.5. Samples were where heat inactivated 1, 5, 10, 30, 60, 120, and 240 min after initiation and then analyzed by ³¹P NMR as described above.

Reactions examining the effect of amino donor concentrations were the same as above, except they contained 1.5, 3, 5, or 10 mM of (L-Asp). Samples were heat inactivated after 120 min and then analyzed by ³¹P NMR as described above.

Biochemical assays of His6-SUMO-PnaA. Typical reaction mixtures (300 μL) contained 20 μM His₆-SUMO-PnaA, 1.5 mM L-PnAla, 3 mM oxaloacetate, and 100 μM pyridoxal 5′-phosphate (PLP) in 50 mM HEPES, 150 mM NaCl, pH 7.5. Reactions were incubated at 30° C. for 2 hours, heat inactivated at 65° C. for 10 minutes, and then analyzed by NMR and LC-MS as described above.

Reactions in time course experiments (150 μL) contained 10 μM His₆-SUMO-PnaA, 1.5 mM L-PnAla, 10 mM oxaloacetate (OAA), and 100 μM pyridoxal 5′-phosphate (PLP) in 50 mM HEPES, 150 mM NaCl, pH7.5. Samples were where heat inactivated 1, 5, 10, 30, 60, 120, and 240 min after initiation and then analyzed by ³¹P NMR as described above.

Reactions examining the effect of keto-acid acceptor concentrations were the same as above, except they contained 1.5, 3, 5, or 10 mM of (OAA). Samples were heat inactivated after 120 min and then analyzed by ³¹P NMR as described above.

Kinetic analyses. All reactions (400 μL) were performed in 50 mM HEPES, 150 mM NaCl, pH 7.5, 30° C. using an Agilent Cary 300 spectrophotometer. The instrument was first blanked using the reaction containing the enzymes and the fixed substrate (without NADH). Initial rates were calculated from continual changes in absorbance (340 nm) before and after substrate addition. Data were analyzed using SigmaPlot 15.

Conversion of PnPy and L-Asp to L-PnAla and OAA by His₆-SUMO-PnaA. Each reaction contained 0.5 μM PnaA, 5 μM MDH, 200 uM NADH, 5 mM L-Asp, and were initiated by the addition of 0.0025, 0.00375, 0.005, 0.0065, 0.008, 0.01, or 0.015 mM PnPy. PnaA was pre-incubated with PLP (1:5) on ice for 10 min prior to use.

Conversion of PEP and L-Asp to L-PnAla and OAA by His₆-SUMO-PnaA and His₆-PnaD. Each reaction contained 1 μM PnaA, 5 μM PnaD, 5 μM MDH, 200 uM NADH, 2 mM MgCl2, 5 mM L-Asp, and were initiated by the addition of 0.1, 0.2, 0.35, 0.5, 0.65, 0.8, or 1 mM PEP. PnaA was pre-incubated with PLP (1:5) on ice for 10 min prior to use.

Conversion of L-PnAla and OAA to PnPy and L-Asp by His₆-SUMO-PnaA. Each reaction contained 1 μM PnaA, 5 μM VlpB, 200 uM NADH, 5 mM OAA, the 340 nm absorbance change was recorded for 1 min to measure the background assumption of OAA by VlpB. Then the reactions were initiated by the addition of 0.1, 0.2, 0.35, 0.5, 0.65, 0.8, or 1 mM PnAla. PnaA was pre-incubated with PLP (1:5) on ice for 10 min prior to use.

Conversion of 2-KG and L-Asp to L-Glu and OAA by His₆-SUMO-PnaA. Each reaction contained 1 μM PnaA, 5 μM MDH, 200 uM NADH, 5 mM L-Asp, and were initiated by the addition of 0.1, 0.2, 0.35, 0.5, 0.65, 0.80, 1, or 1.25 mM 2-KG. PnaA was pre-incubated with PLP (1:5) on ice for 10 min prior to use.

Conversion of L-Glu and OAA to 2-KG and L-Asp by His₆-SUMO-PnaA. Each reaction contained 1 μM PnaA, 5 μM SerA, 200 uM NADH, 5 mM OAA, the 340 nm absorbance change was recorded for 1 min to measure the background assumption of OAA by SerA, and were initiated by the addition of 0.5, 1, 1.5, 2, 3, 4, 6, 10, or 15 mM L-Glu. PnaA was pre-incubated with PLP (1:5) on ice for 10 min prior to use.

Conversion of 2-KG to 2-hydroxyglutaric acid (HGA) by Hisu-SerA. Each reaction contained 1 μM SerA, 200 uM NADH, and were initiated by the addition of 0.02, 0.035, 0.05, 0.075, 0.1, 0.15, 0.2, 0.35 mM 2-KG.

Conversion of OAA to malate by Hisu-SerA. Each reaction contained 1 μM SerA, 200 uM NADH, and were initiated by the addition of 0.05, 0.1, 0.2, 0.375, 0.5, 0.65, 0.8 or 1 mM OAA.

Biochemical assays of His6-PnaB. Typical reaction mixtures (500 L) contained 10 μM His₆-PnaB, 3 mM ATP, 1 mM L-PnAla, 3 mM chemically synthesized L-Ala-L-Val, L-Val-L-Val, L-Ala-L-Ile, or L-Thr-L-Val in 50 mM Tris-HCl, 100 mM NaCl, 2 mM MgCl₂ pH 9. Reactions were incubated at 30° C. for 2 hours, heat inactivated at 65° C. for 10 minutes, and then analyzed by NMR and LC-MS as described above.

Biochemical assays of His6-PnaC. Typical reaction mixtures (100 L) contained 10 μM His₆-PnaC, 3 mM ATP, 3 mM of each L-amino acid, and 5 mM ATP in 50 mM Tris-HCl, 100 mM NaCl, 2 mM MgCl₂ pH 9. Reactions were incubated at 30° C. for 16 hours, heat inactivated at 65° C. for 10 minutes, and then analyzed by LC-MS as described above.

Tripeptide synthesis assays using PnaB and PnaC. Reaction mixtures (200 L) contained 10 μM His₆-PnaB, 10 μM His₆-PnaC, 1 mM L-PnAla, 3 mM L-amino acid A, 3 mM L-amino acid B, and 5 mM ATP in 50 mM Tris-HCl, 100 mM NaCl, 2 mM MgCl₂ pH 9. Reactions were incubated at 30° C. for 16 hours, heat inactivated at 65° C. for 10 minutes, and then analyzed by NMR and LC-MS as described above.

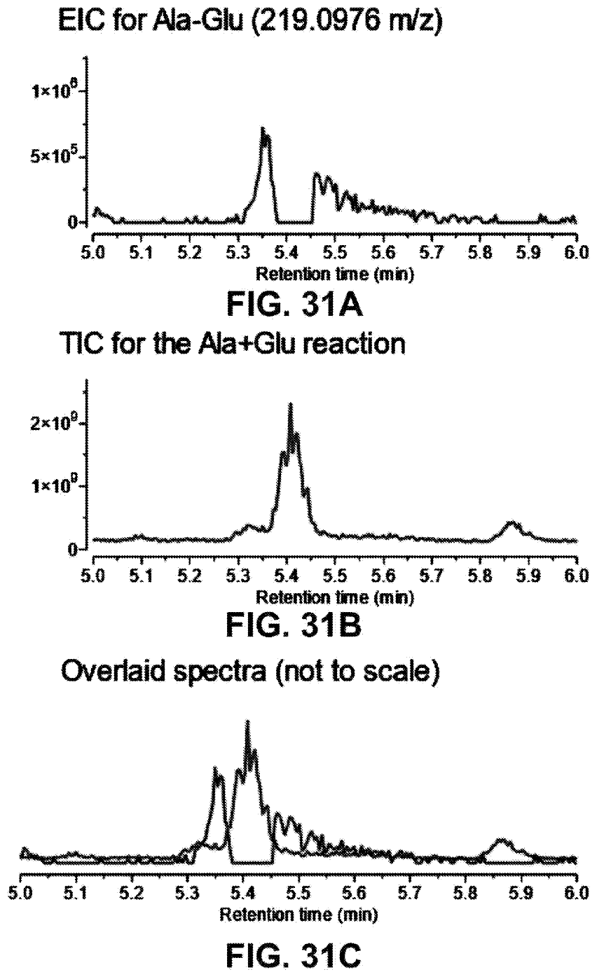

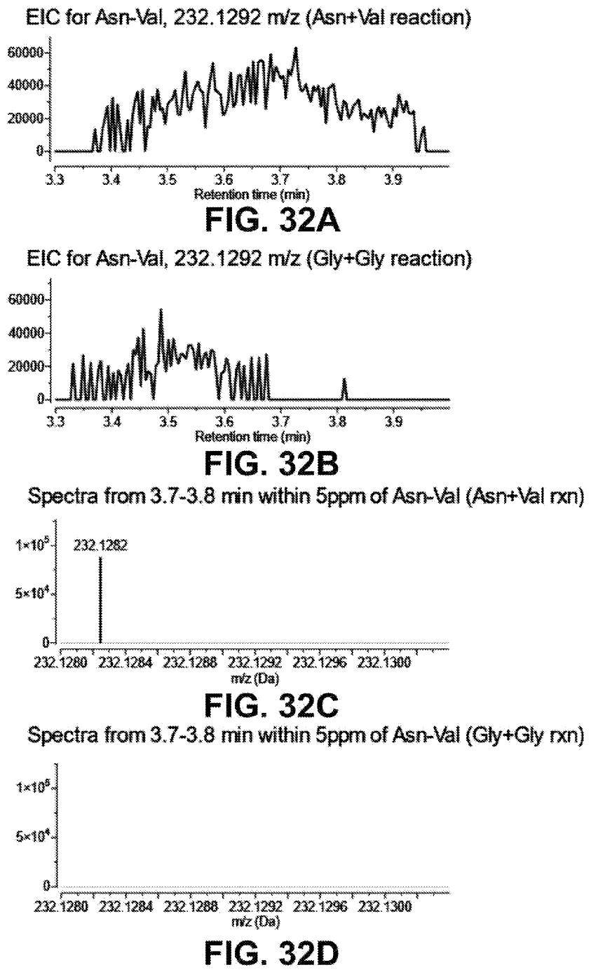

Analysis of PnaC and PnaB substrate specificity reactions. An extracted ion chromatogram (EIC) was obtained for each potential product m z. The shape and retention time of each EIC were used to evaluate the quality and plausibility of true signals, and samples with low-confidence EICs were re-analyzed individually, to address the challenges of ion suppression (FIG. 31A-FIG. 31C) and background noise (FIG. 32A-FIG. 32D).

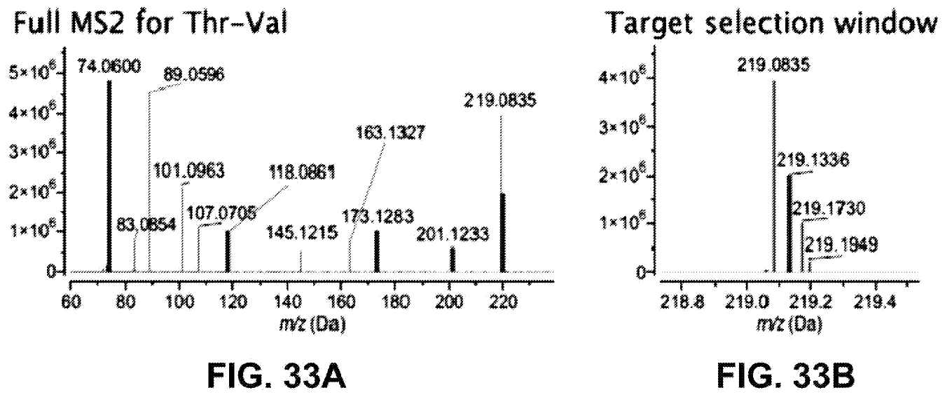

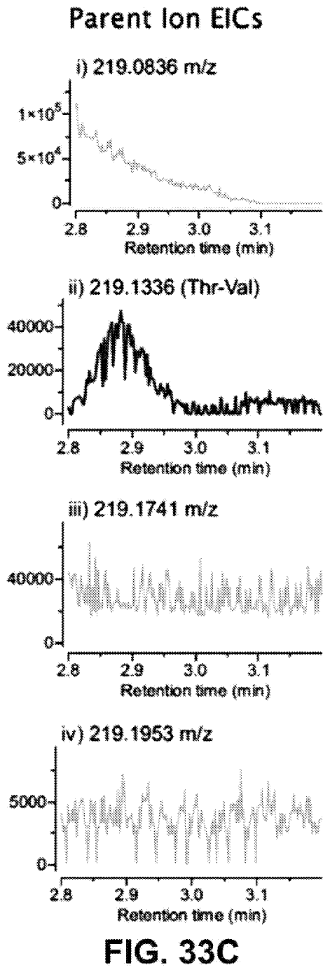

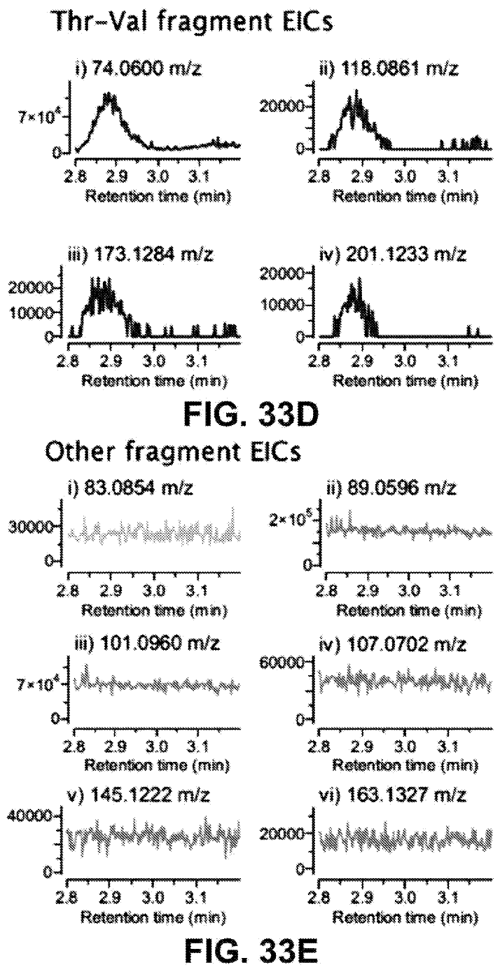

LC-HRMS/MS fragmentation analysis was performed to verify and determine the structure of each product. Published analyses of amino acid fragmentation were referenced in structural assignment of fragment ions [S14]. Fragment assignment was complicated by limitations in parent ion selection, as the Orbitrap instrument used has a minimum isolation window of 0.4 m/z. To determine which fragments resulted from the target parent ion, EICs were compared between each fragment and parent ion (FIG. 33A-FIG. 33E).

### REFERENCES

(B1) Metcalf W W et al. Biosynthesis of phosphonic and phosphinic acid natural products. Annu Rev Biochem 2009, 78, 65-94. DOI: 10.1146/annurev.biochem.78.091707.100215.
(B2) Ju K S et al. Genomics-enabled discovery of phosphonate natural products and their biosynthetic pathways. J Ind Microbiol Biotechnol 2014, 41 (2), 345-356. DOI: 10.1007/s10295-013-1375-2.
(B3) Silver L L. Fosfomycin: Mechanism and Resistance. Cold Spring Harb Perspect Med 2017, 7 (2). DOI: 10.1101/cshperspect.a025262.
(B4) Crumpacker C S. Mechanism of action of foscarnet against viral polymerases. Am J Med 1992, 92 (2A), 3S-7S. DOI: 10.1016/0002-9343(92)90329-a.
(B5) Blodgett J A et al. Conserved biosynthetic pathways for phosalacine, bialaphos and newly discovered phosphonic acid natural products. J Antibiot (Tokyo) 2016, 69 (1), 15-25. DOI: 10.1038/ja.2015.77.
(B6) Knak T et al. Over 40 Years of Fosmidomycin Drug Research: A Comprehensive Review and Future Opportunities. Pharmaceuticals (Basel) 2022, 15 (12). DOI: 10.3390/ph15121553.
(B7) Ju K S et al. Discovery of phosphonic acid natural products by mining the genomes of 10,000 actinomycetes. Proc Natl Acad Sci USA 2015, 112 (39), 12175-12180. DOI: 10.1073/pnas.1500873112.
(B8) Kayrouz C M et al. Genome Mining Reveals the Phosphonoalamide Natural Products and a New Route in Phosphonic Acid Biosynthesis. ACS Chem Biol 2020, 15 (7), 1921-1929. DOI: 10.1021/acschembio.0c00256.
(B9) Wilson J C et al. Discovery of Anti-Phytopathogenic Phosphonopeptide Natural Products from Bacillus velezensis by Genome Mining. [Manuscript submitted for publication] 2023.
(B10) Zhang Y et al. Valinophos Reveals a New Route in Microbial Phosphonate Biosynthesis That Is Broadly Conserved in Nature. J Am Chem Soc 2022, 144 (22), 9938-9948. DOI: 10.1021/jacs.2c02854.
(B11) Zhang Y et al Biosynthesis of Argolaphos Illuminates the Unusual Biochemical Origins of Aminomethylphosphonate and NF-Hydroxyarginine Containing Natural Products. J Am Chem Soc 2022, 144 (22), 9634-9644. DOI: 10.1021/jacs.2c00627.
(B12) Evans B S et al. Discovery of the antibiotic phosacetamycin via a new mass spectrometry-based method for phosphonic acid detection. ACS Chem Biol 2013, 8 (5), 908-913. DOI: 10.1021/cb400102t.
(B13) Polidore A L A et al. A Phosphonate Natural Product Made by Pantoea ananatis is Necessary and Sufficient for the Hallmark Lesions of Onion Center Rot. mBio 2021, 12 (1). DOI: 10.1128/mBio.03402-20.
(B14) Yu X et al. Diversity and abundance of phosphonate biosynthetic genes in nature. Proc Natl Acad Sci USA 2013, 110 (51), 20759-20764. DOI: 10.1073/pnas.1315107110.
(B15) Bowman E et al. Catalysis and thermodynamics of the phosphoenolpyruvate/phosphonopyruvate rearrangement. Entry into the phosphonate class of naturally occurring organophosphorus compounds. Journal of the American Chemical Society 1988, 110 (16), 5575-5576. DOI: 10.1021/ja00224a054.
(B16) Kittredge J S et al. The Occurrence of Alpha-Amino-Beta-Phosphonopropionic Acid in the Zoanthid, Zoanthus Sociatus, and the Ciliate, Tetrahymena Pyriformis. Biochemistry 1964, 3, 991-996. DOI: 10.1021/bi00895a026.
(B17) Hawkinson J E et al. The metabotropic glutamate receptor antagonist L-2-amino-3-phosphonopropionic acid inhibits phosphoserine phosphatase. Eur J Pharmacol 1996, 307 (2), 219-225. DOI: 10.1016/0014-2999(96)00253-1.
(B18) O'Connor J J et al. Long-lasting enhancement of NMDA receptor-mediated synaptic transmission by metabotropic glutamate receptor activation. Nature 1994, 367 (6463), 557-559. DOI: 10.1038/367557a0.
(B19) Schoepp D D et al. Stereoselectivity and mode of inhibition of phosphoinositide-coupled excitatory amino acid receptors by 2-amino-3-phosphonopropionic acid. Mol Pharmacol 1990, 38 (2), 222-228.
(B20) Tan S A T, L. G. Distribution of ciliatine (2-aminoethylphosphonic acid) and phosphonoalanine (2-amino-3-phosphonopropionic acid) in human tissues. Clin Physiol Biochem 1989, 7 (6), 303-309.
(B21) Kulakova A N et al. Expression of the phosphonoalanine-degradative gene cluster from Variovorax sp. Pal2 is induced by growth on phosphonoalanine and phosphonopyruvate. FEMS Microbiol Lett 2009, 292 (1), 100-106. DOI: 10.1111/j.1574-6968.2008.01477.x.
(B22) Ternan N G et al. Initial in vitro characterisation of phosphonopyruvate hydrolase, a novel phosphate starvation-independent, carbon-phosphorus bond cleavage enzyme in Burkholderia cepacia Pal6. Arch Microbiol 2000, 173 (1), 35-41. DOI: 10.1007/s002030050005.
(B23) Kulakova A N et al. The purification and characterization of phosphonopyruvate hydrolase, a novel carbon-phosphorus bond cleavage enzyme from Variovorax sp Pal2. J Biol Chem 2003, 278 (26), 23426-23431. DOI: 10.1074/jbc.M301871200 From NLM Medline.
(B24) Chin J P et al. Microbial transformations in phosphonate biosynthesis and catabolism, and their importance in nutrient cycling. Curr Opin Chem Biol 2016, 31, 50-57. DOI: 10.1016/j.cbpa.2016.01.010.
(B25) Metcalf W W et al. Synthesis of methylphosphonic acid by marine microbes: a source for methane in the aerobic ocean. Science 2012, 337 (6098), 1104-1107. DOI: 10.1126/science.1219875.
(B26) Toney M D. Aspartate aminotransferase: an old dog teaches new tricks. Arch Biochem Biophys 2014, 544, 119-127. DOI: 10.1016/j.abb.2013.10.002 From NLM Medline.
(B27) Zhang G et al. The phosphonopyruvate decarboxylase from Bacteroides fragilis. J Biol Chem 2003, 278 (42), 41302-41308. DOI: 10.1074/jbc.M305976200.
(B28) Nakashita H et al. Studies on the Biosynthesis of Bialaphos. Biochemical Mechanism of C—P Bond Formation: Discovery of Phosphonopyruvate Decarboxylase which Catalyzes the Formation of Phosphonoacetaldehyde from Phosphonopyruvate. The Journal of Antibiotics 1997, 50 (3), 212-219. DOI: 10.7164/antibiotics.50.212.
(B29) Eliot A C et al. Cloning, expression, and biochemical characterization of Streptomyces rubellomurinus genes required for biosynthesis of antimalarial compound FR900098. Chem Biol 2008, 15 (8), 765-770. DOI: 10.1016/j.chembiol.2008.07.010.
(B30) Kim J et al. Phosphoenolpyruvate mutase catalysis of phosphoryl transfer in phosphoenolpyruvate: kinetics and mechanism of phosphorus-carbon bond formation. Biochemistry 1996, 35 (14), 4628-4635. DOI: 10.1021/bi952944k.
(B31) Kuramitsu S et al. Pre-steady-state kinetics of Escherichia coli aspartate aminotransferase catalyzed reactions and thermodynamic aspects of its substrate specificity. Biochemistry 1990, 29 (23), 5469-5476. DOI: 10.1021/bi00475a010 From NLM Medline.
(B32) Fawaz M V et al. The ATP-grasp enzymes. Bioorg Chem 2011, 39 (5-6), 185-191. DOI: 10.1016/j.bioorg.2011.08.004 From NLM Medline.
(B33) Ogasawara Y et al. Biosynthesis of Oligopeptides Using ATP-Grasp Enzymes. Chemistry 2017, 23 (45), 10714-10724. DOI: 10.1002/chem.201700674 From NLM Medline.
(B34) Borisova S A et al. Biosynthesis of rhizocticins, antifungal phosphonate oligopeptides produced by Bacillus subtilis ATCC6633. Chem Biol 2010, 17 (1), 28-37. DOI: 10.1016/j.chembiol.2009.11.017.
(B35) Senoo A et al. Identification of novel L-amino acid alpha-ligases through Hidden Markov Model-based profile analysis. Biosci Biotechnol Biochem 2010, 74 (2), 415-418. DOI: 10.1271/bbb.90644.
(B36) Tabata K et al. ywfE in Bacillus subtilis codes for a novel enzyme, L-amino acid ligase. J Bacteriol 2005, 187 (15), 5195-5202. DOI: 10.1128/JB.187.15.5195-5202.2005.
(B37) Arai T et al. L-amino acid ligase from Pseudomonas syringae producing tabtoxin can be used for enzymatic synthesis of various functional peptides. Appl Environ Microbiol 2013, 79 (16), 5023-5029. DOI: 10.1128/AEM.01003-13.
(B38) Arai T et al. A novel L-amino acid ligase is encoded by a gene in the phaseolotoxin biosynthetic gene cluster from Pseudomonas syringae pv. phaseolicola 1448A. Biosci Biotechnol Biochem 2008, 72 (11), 3048-3050. DOI: 10.1271/bbb.80439.
(B39) Kino K et al. A novel L-amino acid ligase from Bacillus subtilis NBRC3134 catalyzed oligopeptide synthesis. Biosci Biotechnol Biochem 2010, 74 (1), 129-134. DOI: 10.1271/bbb.90649.
(B40) Kino K et al. A novel L-amino acid ligase from Bacillus subtilis NBRC3134, a microorganism producing peptide-antibiotic rhizocticin. Biosci Biotechnol Biochem 2009, 73 (4), 901-907. DOI: 10.1271/bbb.80842.
(B41) Kino K et al. Dipeptide synthesis by L-amino acid ligase from Ralstonia solanacearum. Biochem Biophys Res Commun 2008, 371 (3), 536-540. DOI: 10.1016/j.bbrc.2008.04.105.
(B42) Kino K et al. Identification and characterization of a novel L-amino acid ligase from Photorhabdus luminescens subsp. laumondii TTO1. J Biosci Bioeng 2010, 110 (1), 39-41. DOI: 10.1016/j.jbiosc.2009.12.004.
(B43) Kino K et al. A novel 1-amino acid ligase from Bacillus licheniformis. J Biosci Bioeng 2008, 106 (3), 313-315. DOI: 10.1263/jbb.106.313.
(B44) Wang T et al. 1-amino acid ligase: A promising alternative for the biosynthesis of 1-dipeptides. Enzyme Microb Technol 2020, 136, 109537. DOI: 10.1016/j.enzmictec.2020.109537.
(B45) Pederick J L et al. Discovery of an L-amino acid ligase implicated in Staphylococcal sulfur amino acid metabolism. J Biol Chem 2022, 102392. DOI: 10.1016/j.jbc.2022.102392.
(B46) Saio T et al. Ligand-driven conformational changes of MurD visualized by paramagnetic NMR. Sci Rep 2015, 5, 16685. DOI: 10.1038/srep16685.
(B47) Tabata K et al. Fermentative production of L-alanyl-L-glutamine by a metabolically engineered Escherichia coli strain expressing L-amino acid alpha-ligase. Appl Environ Microbiol 2007, 73 (20), 6378-6385. DOI: 10.1128/AEM.01249-07.
(B48) Wentzel A et al. Intracellular Metabolite Pool Changes in Response to Nutrient Depletion Induced Metabolic Switching in Streptomyces coelicolor. Metabolites 2012, 2 (1), 178-194. DOI: 10.3390/metabo2010178.
(B49) Bennett B D et al. Absolute metabolite concentrations and implied enzyme active site occupancy in Escherichia coli. Nat Chem Biol 2009, 5 (8), 593-599. DOI: 10.1038/nchembio.186.
(B50) Rahman M et al. Growth phase-dependent changes in the expression of global regulatory genes and associated metabolic pathways in Escherichia coli. Biotechnol Lett 2008, 30 (5), 853-860. DOI: 10.1007/s10529-007-9621-1.
(B51) Park B K et al. Structure of Plumbemycin A and B, Antagonists of l-Threonine from Streptomyces plumbeus. Agricultural and Biological Chemistry 1977, 41 (3), 573-579. DOI: 10.1080/00021369.1977.10862538.
(B52) Blodgett J A et al. Molecular cloning, sequence analysis, and heterologous expression of the phosphinothricin tripeptide biosynthetic gene cluster from Streptomyces viridochromogenes DSM 40736. Antimicrob Agents Chemother 2005, 49 (1), 230-240. DOI: 10.1128/AAC.49.1.230-240.2005.
(B53) Circello B T et al. Molecular cloning and heterologous expression of the dehydrophos biosynthetic gene cluster. Chem Biol 2010, 17 (4), 402-411. DOI: 10.1016/j.chembiol.2010.03.007.
(B54) Huang Z et al. New Insights into the Biosynthesis of Fosfazinomycin. Chem Sci 2016, 7 (8), 5219-5223. DOI: 10.1039/C6SC01389A.
(B55) Huang Z et al. Biosynthesis of fosfazinomycin is a convergent process. Chem Sci 2015, 6 (2), 1282-1287. DOI: 10.1039/C4SC03095H.
(B56) Circello B T et al. The antibiotic dehydrophos is converted to a toxic pyruvate analog by peptide bond cleavage in Salmonella enterica. Antimicrob Agents Chemother 2011, 55 (7), 3357-3362. DOI: 10.1128/AAC.01483-10.
(B57) Kugler M et al. Rhizocticin A, an antifungal phosphono-oligopeptide of Bacillus subtilis ATCC 6633: biological properties. Arch Microbiol 1990, 153 (3), 276-281.
(B58) Abouhamad W N et al. Peptide transport and chemotaxis in Escherichia coli and Salmonella typhimurium: characterization of the dipeptide permease (Dpp) and the dipeptide-binding protein. Mol Microbiol 1991, 5 (5), 1035-1047. DOI: 10.1111/j.1365-2958.1991.tb01876.x.
(B59) Wencewicz T A et al. Pseudomonas syringae self-protection from tabtoxinine-beta-lactam by ligase TblF and acetylase Ttr. Biochemistry 2012, 51 (39), 7712-7725. DOI: 10.1021/bi3011384.
(B60) Tsuda T et al. Single mutation alters the substrate specificity of L-amino acid ligase. Biochemistry 2014, 53 (16), 2650-2660. DOI: 10.1021/bi500292b.
S1 Gerhardt P M. Et al. Methods for General and Molecular Bacteriology. 2nd edn, (American Society for Microbiology, 1994).
S2 Stanier R Y et al. The aerobic pseudomonads: a taxonomic study. J Gen Microbiol 43, 159-271, doi:10.1099/00221287-43-2-159 (1966).
S3 Green J R S, J. Molecular Cloning. 4th edn, (Cold Spring Harbor Laboratory Press, 2012).
S4 Zhang Y et al. Biosynthesis of Argolaphos Illuminates the Unusual Biochemical Origins of Aminomethylphosphonate and Nε-Hydroxyarginine Containing Natural Products. J Am Chem Soc 144, 9634-9644, doi:10.1021/jacs.2c00627 (2022).
S5 Wick R R et al. Unicycler: Resolving bacterial genome assemblies from short and long sequencing reads. PLoS Comput Biol 13, e1005595, doi:10.1371/journal.pcbi.1005595 (2017).
S6 Tatusova T et al. NCBI prokaryotic genome annotation pipeline. Nucleic Acids Res 44, 6614-6624, doi:10.1093/nar/gkw569 (2016).
S7 Mistry J et al. Pfam: The protein families database in 2021. Nucleic Acids Res 49, D412-D419, doi:10.1093/nar/gkaa913 (2021).
S8 Wheeler D L et al. Database resources of the National Center for Biotechnology. Nucleic Acids Res 31, 28-33, doi:10.1093/nar/gkg033 (2003).
S9 Lu S et al. CDD/SPARCLE: the conserved domain database in 2020. Nucleic Acids Res 48, D265-D268, doi:10.1093/nar/gkz991 (2020).
S10 Sullivan M J et al. Easyfig: a genome comparison visualizer. Bioinformatics 27, 1009-1010, doi:10.1093/bioinformatics/btr039 (2011).
S11 Datsenko K A et al. One-step inactivation of chromosomal genes in Escherichia coli K-12 using PCR products. Proc Natl Acad Sci USA 97, 6640-6645, doi:10.1073/pnas.120163297 (2000).
S12 Circello B T et al. Molecular cloning and heterologous expression of the dehydrophos biosynthetic gene cluster. Chem Biol 17, 402-411, doi:10.1016/j.chembiol.2010.03.007 (2010).
S13 Zhang Y et al. Valinophos Reveals a New Route in Microbial Phosphonate Biosynthesis That Is Broadly Conserved in Nature. J Am Chem Soc 144, 9938-9948, doi:10.1021/jacs.2c02854 (2022).
S14 Zhang P et al. Revisiting Fragmentation Reactions of Protonated alpha-Amino Acids by High-Resolution Electrospray Ionization Tandem Mass Spectrometry with Collision-Induced Dissociation. Sci Rep 9, 6453, doi:10.1038/s41598-019-42777-8 (2019).
S15 Eliot A C et al. Cloning, expression, and biochemical characterization of Streptomyces rubellomurinus genes required for biosynthesis of antimalarial compound FR900098. Chem Biol 15, 765-770, doi:10.1016/j.chembiol.2008.07.010 (2008).
S16 Yu X et al. Purification and characterization of phosphonoglycans from Glycomyces sp. strain NRRL B-16210 and Stackebrandtia nassauensis NRRL B-16338. J Bacteriol 196, 1768-1779, doi:10.1128/JB.00036-14 (2014).

### Exemplary Aspects

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: A compound defined by Formula I:

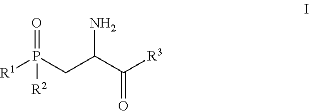

wherein
R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b};
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

Example 2: The compound of any examples herein, particularly example 1, wherein R¹ is OR^{a} and/or R² is OR^{b}.

Example 3: The compound of any examples herein, particularly example 1 or example 2, wherein the compound is defined by Formula II:

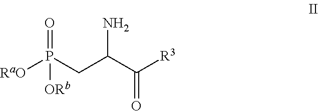

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

Example 4: The compound of any examples herein, particularly examples 1-3, wherein R^{a} and/or R^{b} is hydrogen.

Example 5: The compound of any examples herein, particularly examples 1-4, wherein the compound is defined by Formula III:

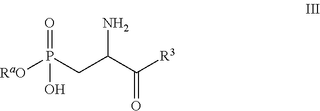

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids);
R^{a} is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

Example 6: The compound of any examples herein, particularly examples 1-5, wherein the compound is defined by Formula IV:

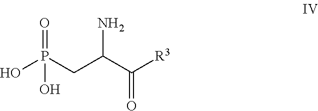

wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more amino acids (e.g., one or more canonical or non-canonical amino acids); and
R^{x} and R^{y} are independently selected from hydrogen, or substituted or unsubstituted C₁-C₅ alkyl, or substituted or unsubstituted C₁-C₅ acyl;
or a derivative or salt thereof,
with the proviso that the compound is not phosphonoalamide A, B, C, D, E, or F.

Example 7: The compound of any examples herein, particularly examples 1-6, wherein R³ is one or more amino acids (e.g., one or more canonical or non-canonical amino acids).

Example 8: The compound of any examples herein, particularly examples 1-7, wherein R³ is one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

Example 9: The compound of any examples herein, particularly examples 1-8, wherein the compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof.

Example 10: The compound of any examples herein, particularly examples 1-9, wherein the compound comprises PnAla-Ala-Val, PnAla-Val-Val, PnAla-Ala-Ile, PnAla-Thr-Val, a derivative or salt thereof, or a combination thereof.

Example 11: The compound of any examples herein, particularly examples 1-9, wherein the compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof.

Example 12: The compound of any examples herein, particularly examples 1-11, wherein the compound comprises PnAla-Ala-Ala, PnAla-Ala-Val, PnAla-Ala-Ile, PnAla-Ala-Leu, PnAla-Ala-Met, PnAla-Ala-Phe, PnAla-Ala-Tyr, PnAla-Ala-Trp, PnAla-Ala-Ser, PnAla-Ala-Thr, PnAla-Ala-aThr, PnAla-Ala-Asn, PnAla-Ala-Gln, PnAla-Ala-Arg, PnAla-Ala-His, PnAla-Ala-Lys, PnAla-Ala-Glu, PnAla-Ala-Gly, PnAla-Ala-Pro, PnAla-Val-Val, PnAla-Val-Ile, PnAla-Val-Leu, PnAla-Val-Met, PnAla-Val-Phe, PnAla-Val-Tyr, PnAla-Val-Trp, PnAla-Val-aThr, PnAla-Val-Gln, PnAla-Val-Pro, PnAla-Ile-Ile, PnAla-Ile-Phe, PnAla-Ile-aThr, PnAla-Leu-Leu, PnAla-Met-Val, PnAla-Met-Leu, PnAla-Met-Met, PnAla-Met-Trp, PnAla-Phe-Val, PnAla-Phe-Ile, PnAla-Phe-Leu, PnAla-Phe-Met, PnAla-Phe-Phe, PnAla-Phe-Tyr, PnAla-Phe-Trp, PnAla-Phe-Thr, PnAla-Phe-aThr, PnAla-Tyr-Val, PnAla-Tyr-Leu, PnAla-Tyr-Met, PnAla-Tyr-Phe, PnAla-Tyr-Tyr, PnAla-Tyr-Trp, PnAla-Tyr-Gly, PnAla-Trp-Val, PnAla-Trp-Ile, PnAla-Trp-Leu, PnAla-Trp-Met, PnAla-Trp-Phe, PnAla-Trp-Tyr, PnAla-Trp-Trp, PnAla-Trp-Thr, PnAla-Trp-Lys, PnAla-Trp-Gly, PnAla-Ser-Val, PnAla-Ser-Ile, PnAla-Ser-Leu, PnAla-Ser-Met, PnAla-Ser-Phe, PnAla-Ser-Tyr, PnAla-Ser-Ser, PnAla-Ser-Thr, PnAla-Ser-aThr, PnAla-Thr-Val, PnAla-Thr-Ile, PnAla-Thr-Leu, PnAla-Thr-Phe, PnAla-Thr-Trp, PnAla-aThr-Val, PnAla-aThr-Ile, PnAla-aThr-Leu, PnAla-aThr-Met, PnAla-aThr-Phe, PnAla-aThr-aThr, PnAla-Gln-Val, PnAla-Gln-Ile, PnAla-Gln-Leu, PnAla-His-Phe, PnAla-His-Trp, PnAla-Gly-Val, PnAla-Gly-Ile, PnAla-Gly-Leu, PnAla-Gly-Met, PnAla-Gly-Phe, PnAla-Gly-Tyr, PnAla-Gly-Trp, PnAla-Gly-aThr, PnAla-Gly-Gly, a derivative or salt thereof, or a combination thereof.

Example 13: The compound of any examples herein, particularly examples 1-12, wherein the compound is a salt.

Example 14: The compound of any examples herein, particularly examples 1-13, wherein the compound is a salt form of Formula I, Formula II, Formula III, Formula IV, or a combination thereof with a counterion.

Example 15: The compound of any examples herein, particularly examples 1-14, wherein the compound is a salt form of Formula II with a counterion.

Example 16: The compound of any examples herein, particularly examples 1-15, wherein the compound is a salt form of Formula II with a counterion and the salt form of the compound is selected from the group consisting of:

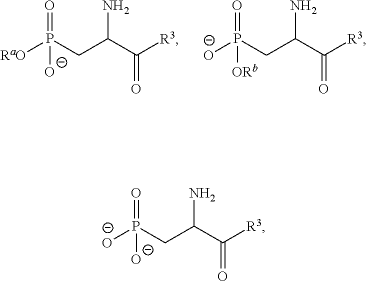

and combinations thereof.

Example 17: The compound of any examples herein, particularly examples 14-16, wherein the counterion is a monovalent or divalent counterion.

Example 18: The compound of any examples herein, particularly examples 14-17, wherein the counterion is selected from the group consisting of sodium, potassium, calcium, lithium, magnesium, manganese, ammonium, iron, and combinations thereof.

Example 19: The compound of any examples herein, particularly examples 1-18, wherein the compound is a potassium salt, sodium salt, calcium salt, iron salt, ammonium salt, or a combination thereof.

Example 20: The compound of any examples herein, particularly examples 1-19, wherein the compound comprises an agriculturally acceptable salt thereof and/or a pharmaceutically acceptable salt thereof.

Example 21: A compound comprising a head group and a tail group, the head group being bound to the tail group using one or more enzymes derived from Streptomyces, wherein the head group comprises a phosphonic acid, a phosphinic acid, or a derivative thereof, and the tail group comprises one or more (canonical or non-canonical) amino acids, with the proviso that the compound is not phosphonoalamide A, B, C, D, E, or F.

Example 22: The compound of any examples herein, particularly example 21, wherein the head group comprises phosphonoalanine (PnAla) or a derivative thereof.

Example 23: The compound of any examples herein, particularly example 21 or example 22, wherein the tail group comprises one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

Example 24: The compound of any examples herein, particularly examples 21-23, wherein the compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof.

Example 25: The compound of any examples herein, particularly examples 21-24, wherein the compound comprises PnAla-Ala-Val, PnAla-Val-Val, PnAla-Ala-Ile, PnAla-Thr-Val, or a combination thereof.

Example 26: The compound of any examples herein, particularly examples 21-25, wherein the compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof.

Example 27: The compound of any examples herein, particularly examples 21-26, wherein the compound comprises PnAla-Ala-Ala, PnAla-Ala-Val, PnAla-Ala-Ile, PnAla-Ala-Leu, PnAla-Ala-Met, PnAla-Ala-Phe, PnAla-Ala-Tyr, PnAla-Ala-Trp, PnAla-Ala-Ser, PnAla-Ala-Thr, PnAla-Ala-aThr, PnAla-Ala-Asn, PnAla-Ala-Gln, PnAla-Ala-Arg, PnAla-Ala-His, PnAla-Ala-Lys, PnAla-Ala-Glu, PnAla-Ala-Gly, PnAla-Ala-Pro, PnAla-Val-Val, PnAla-Val-Ile, PnAla-Val-Leu, PnAla-Val-Met, PnAla-Val-Phe, PnAla-Val-Tyr, PnAla-Val-Trp, PnAla-Val-aThr, PnAla-Val-Gln, PnAla-Val-Pro, PnAla-Ile-Ile, PnAla-Ile-Phe, PnAla-Ile-aThr, PnAla-Leu-Leu, PnAla-Met-Val, PnAla-Met-Leu, PnAla-Met-Met, PnAla-Met-Trp, PnAla-Phe-Val, PnAla-Phe-Ile, PnAla-Phe-Leu, PnAla-Phe-Met, PnAla-Phe-Phe, PnAla-Phe-Tyr, PnAla-Phe-Trp, PnAla-Phe-Thr, PnAla-Phe-aThr, PnAla-Tyr-Val, PnAla-Tyr-Leu, PnAla-Tyr-Met, PnAla-Tyr-Phe, PnAla-Tyr-Tyr, PnAla-Tyr-Trp, PnAla-Tyr-Gly, PnAla-Trp-Val, PnAla-Trp-Ile, PnAla-Trp-Leu, PnAla-Trp-Met, PnAla-Trp-Phe, PnAla-Trp-Tyr, PnAla-Trp-Trp, PnAla-Trp-Thr, PnAla-Trp-Lys, PnAla-Trp-Gly, PnAla-Ser-Val, PnAla-Ser-Ile, PnAla-Ser-Leu, PnAla-Ser-Met, PnAla-Ser-Phe, PnAla-Ser-Tyr, PnAla-Ser-Ser, PnAla-Ser-Thr, PnAla-Ser-aThr, PnAla-Thr-Val, PnAla-Thr-Ile, PnAla-Thr-Leu, PnAla-Thr-Phe, PnAla-Thr-Trp, PnAla-aThr-Val, PnAla-aThr-Ile, PnAla-aThr-Leu, PnAla-aThr-Met, PnAla-aThr-Phe, PnAla-aThr-aThr, PnAla-Gln-Val, PnAla-Gln-Ile, PnAla-Gln-Leu, PnAla-His-Phe, PnAla-His-Trp, PnAla-Gly-Val, PnAla-Gly-Ile, PnAla-Gly-Leu, PnAla-Gly-Met, PnAla-Gly-Phe, PnAla-Gly-Tyr, PnAla-Gly-Trp, PnAla-Gly-aThr, PnAla-Gly-Gly, or a combination thereof.

Example 28: The compound of any examples herein, particularly examples 21-27, wherein the head group is N-terminal.

Example 29: The compound of any examples herein, particularly examples 21-28, wherein the compound is a di-peptide or a tri-peptide.

Example 30: The compound of any examples herein, particularly examples 21-29, wherein the compound is a salt.

Example 31: The compound of any examples herein, particularly examples 21-30, wherein the compound is a potassium salt, sodium salt, calcium salt, iron salt, ammonium salt, or a combination thereof.

Example 32: The compound of any examples herein, particularly examples 21-31, wherein the compound comprises an agriculturally acceptable salt thereof and/or a pharmaceutically acceptable salt thereof.

Example 33: The compound of any examples herein, particularly examples 21-32, wherein the compound is of any examples herein, particularly examples 1-19.

Example 34: The compound of any examples herein, particularly examples 1-33, wherein the compound is a Streptomyces isolate or a derivative or salt thereof.

Example 35: A composition comprising the compound of any examples herein, particularly examples 1-34.

Example 36: The composition of any examples herein, particularly example 35, wherein the composition further comprises one or more agriculturally acceptable and/or pharmaceutically acceptable carriers.

Example 37: The composition of any examples herein, particularly example 35 or example 36, wherein the composition comprises a pharmaceutical composition, an agricultural composition, or a combination thereof.

Example 38: The composition of any examples herein, particularly examples 35-37, wherein the composition comprises a pesticide.

Example 39: The composition of any examples herein, particularly examples 35-38, wherein the composition comprises an herbicide.

Example 40: The composition of any examples herein, particularly examples 35-39, wherein the composition exhibits antimicrobial activity.

Example 41: The composition of any examples herein, particularly examples 35-40, wherein the composition results in at least 5 log reduction of a population of microbes.

Example 42: The composition of any examples herein, particularly examples 35-41, further comprising a solvent, a carrier, an excipient, or a combination thereof.

Example 43: The composition of any examples herein, particularly examples 35-42, further comprising an agriculturally acceptable adjuvant or carrier.

Example 44: The composition of any examples herein, particularly examples 35-43, wherein the composition is formulated for delivery to a plant or animal.

Example 45: The composition of any examples herein, particularly examples 35-44, wherein the composition is formulated for delivery to a plant.

Example 46: The composition of any examples herein, particularly example 45, wherein the plant comprises a crop.

Example 47: The composition of any examples herein, particularly examples 35-44, wherein the composition is formulated for delivery to an animal.

Example 48: The composition of any examples herein, particularly example 47, wherein the animal is a companion animal, livestock, research animal, insect, or human.

Example 49: A nucleic acid encoding the compound or composition of any examples herein, particularly examples 1-48.

Example 50: A vector encoding the nucleic acid of any examples herein, particularly example 49.

Example 51: A cell comprising the vector of any examples herein, particularly example 50.

Example 52: A cell comprising the compound or composition of any examples herein, particularly examples 1-48.

Example 53: The cell of any examples herein, particularly example 51 or example 52, wherein the cell comprises a Streptomyces cell.

Example 54: A method of making the compound of any examples herein, particularly examples 1-34.

Example 55: The method of any examples herein, particularly example 54, wherein the method is a biosynthetic method.

Example 56: The method of any examples herein, particularly example 54 or example 55, wherein the method uses one or more enzymes derived from Streptomyces.

Example 57: A method of making a compound comprising a head group and a tail group, the head group being bound to the tail group, wherein the head group comprises a phosphonic acid, a phosphinic acid, or a derivative thereof, and the tail group comprises one or more (canonical or non-canonical) amino acids, using one or more enzymes derived from Streptomyces, with the proviso that the compound is not phosphonoalamide A, B, C, D, E, or F.

Example 58: The method of any examples herein, particularly example 57, wherein the head group comprises PnAla or a derivative thereof.

Example 59: The method of any examples herein, particularly example 57 or example 58, wherein the tail group comprises one or more amino acids, each amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

Example 60: The method of any examples herein, particularly examples 57-59, wherein the compound is the compound of any examples herein, particularly examples 1-34.

Example 61: The method of any examples herein, particularly examples 56-60, wherein the one or more enzymes comprise one or more ATP-grasp enzymes.

Example 62: The method of any examples herein, particularly examples 56-61, wherein the one or more enzymes are encoded by a gene comprising at least 90% identity to pnaB, pnaC, or a combination thereof.

Example 63: The method of any examples herein, particularly examples 56-62, wherein the method proceeds via a convergent pathway.

Example 64: The method of any examples herein, particularly examples 54-63, wherein the method comprises contacting a first amino acid and a second amino acid with a first enzyme, to thereby form a first compound comprising the first amino acid bound to the second amino acid.

Example 65: The method of any examples herein, particularly example 64, wherein the first enzyme is encoded by a gene comprising at least 90% identity to pnaC.

Example 66: The method of any examples herein, particularly example 64 or example 65, wherein the first enzyme comprises PnaC.

Example 67: The method of any examples herein, particularly examples 64-66, wherein the first amino acid and/or the second amino acid each independently comprises Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), Valine (Val), a derivative thereof, or a combination thereof.

Example 58: The method of any examples herein, particularly examples 64-67, wherein the first compound comprises Ala-Gly, Ala-Ala, Ala-Val, Ala-Ser, Ala-Thr, Ala-Cys, Ala-Leu, Ala-Ile, Ala-Met, Ala-Asn, Ala-Gln, Ala-Lys, Ala-Arg, Ala-Phe, Ala-Trp, Ala-Tyr, Ala-His, Gly-Gly, Gly-Val, Gly-Leu, Gly-Ile, Ser-Gly, Ser-Val, Ser-Ser, Ser-Leu, Ser-Ile, Ser-Met, Val-Val, Met-Met, Ala-L-allo-Thr, Ser-L-allo-Thr, or a combination thereof.

Example 69: The method of any examples herein, particularly examples 54-68, wherein the method comprises contacting: a third amino acid or the first compound, and a carboxylate with a second enzyme, the first compound being a nucleophile, the carboxylate comprising a phosphonic acid, a phosphinic acid, or a derivative thereof, to thereby form a second compound comprising the third amino acid or the first compound bound to the carboxylate.

Example 70: The method of any examples herein, particularly example 69, wherein the second enzyme is encoded by a gene comprising at least 90% identity to pnaB.

Example 71: The method of any examples herein, particularly example 69 or example 70, wherein the second enzyme comprises PnaB.

Example 72: The method of any examples herein, particularly examples 69-71, wherein the carboxylate comprises phosphonoalanine (e.g., PnAla).

Example 73: The method of any examples herein, particularly examples 69-72, wherein the second compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof.

Example 74: The method of any examples herein, particularly examples 69-73, wherein the second compound comprises PnAla-Ala-Val, PnAla-Val-Val, PnAla-Ala-Ile, PnAla-Thr-Val, or a combination thereof.

Example 75: The method of any examples herein, particularly examples 69-73, wherein the second compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, or a combination thereof.

Example 76: The method of any examples herein, particularly examples 69-75, wherein the second compound comprises PnAla-Ala-Ala, PnAla-Ala-Val, PnAla-Ala-Ile, PnAla-Ala-Leu, PnAla-Ala-Met, PnAla-Ala-Phe, PnAla-Ala-Tyr, PnAla-Ala-Trp, PnAla-Ala-Ser, PnAla-Ala-Thr, PnAla-Ala-aThr, PnAla-Ala-Asn, PnAla-Ala-Gln, PnAla-Ala-Arg, PnAla-Ala-His, PnAla-Ala-Lys, PnAla-Ala-Glu, PnAla-Ala-Gly, PnAla-Ala-Pro, PnAla-Val-Val, PnAla-Val-Ile, PnAla-Val-Leu, PnAla-Val-Met, PnAla-Val-Phe, PnAla-Val-Tyr, PnAla-Val-Trp, PnAla-Val-aThr, PnAla-Val-Gln, PnAla-Val-Pro, PnAla-Ile-Ile, PnAla-Ile-Phe, PnAla-Ile-aThr, PnAla-Leu-Leu, PnAla-Met-Val, PnAla-Met-Leu, PnAla-Met-Met, PnAla-Met-Trp, PnAla-Phe-Val, PnAla-Phe-Ile, PnAla-Phe-Leu, PnAla-Phe-Met, PnAla-Phe-Phe, PnAla-Phe-Tyr, PnAla-Phe-Trp, PnAla-Phe-Thr, PnAla-Phe-aThr, PnAla-Tyr-Val, PnAla-Tyr-Leu, PnAla-Tyr-Met, PnAla-Tyr-Phe, PnAla-Tyr-Tyr, PnAla-Tyr-Trp, PnAla-Tyr-Gly, PnAla-Trp-Val, PnAla-Trp-Ile, PnAla-Trp-Leu, PnAla-Trp-Met, PnAla-Trp-Phe, PnAla-Trp-Tyr, PnAla-Trp-Trp, PnAla-Trp-Thr, PnAla-Trp-Lys, PnAla-Trp-Gly, PnAla-Ser-Val, PnAla-Ser-Ile, PnAla-Ser-Leu, PnAla-Ser-Met, PnAla-Ser-Phe, PnAla-Ser-Tyr, PnAla-Ser-Ser, PnAla-Ser-Thr, PnAla-Ser-aThr, PnAla-Thr-Val, PnAla-Thr-Ile, PnAla-Thr-Leu, PnAla-Thr-Phe, PnAla-Thr-Trp, PnAla-aThr-Val, PnAla-aThr-Ile, PnAla-aThr-Leu, PnAla-aThr-Met, PnAla-aThr-Phe, PnAla-aThr-aThr, PnAla-Gln-Val, PnAla-Gln-Ile, PnAla-Gln-Leu, PnAla-His-Phe, PnAla-His-Trp, PnAla-Gly-Val, PnAla-Gly-Ile, PnAla-Gly-Leu, PnAla-Gly-Met, PnAla-Gly-Phe, PnAla-Gly-Tyr, PnAla-Gly-Trp, PnAla-Gly-aThr, PnAla-Gly-Gly, or a combination thereof.

Example 77: The method of any examples herein, particularly examples 61-76, wherein the method further performed in the presence of ATP.

Example 78: A method of use of the compound, composition, nucleic acid, vector, or cell of any examples herein, particularly examples 1-53.

Example 79: The method of any examples herein, particularly example 78, wherein the method comprises using the compound, composition, nucleic acid, vector, or cell as an antimicrobial, herbicide, pesticide, or combination thereof to control an undesirable population.

Example 80: The method of any examples herein, particularly example 79, wherein the method comprises using the compound, composition, nucleic acid, vector, or cell as a pesticide.

Example 81: The method of any examples herein, particularly example 80, wherein the method comprises using the compound, composition, nucleic acid, vector, or cell to control an undesirable population in plants.

Example 82: The method of any examples herein, particularly example 81, wherein the method comprises contacting the plants or the locus thereof with or applying to the soil or water the compound, composition, nucleic acid, vector, or cell.

Example 83: The method of any examples herein, particularly examples 80-82, further comprising applying an additional pesticide.

Example 84: The method of any examples herein, particularly examples 79-83, wherein the undesirable population is an herbicide resistant or tolerant population, a pesticide resistant or tolerant population, an antimicrobial resistant or tolerant population, or a combination thereof.

Example 85: The method of any examples herein, particularly examples 79-84, wherein the undesirable population comprises bacteria.

Example 86: A method of reducing the activity of bacteria, the method comprising exposing the bacteria to an effective amount of the compound, composition, nucleic acid, vector, or cell of any examples herein, particularly examples 1-53.

Example 87: A method of reducing bacterial population, the method comprising exposing the bacteria to an effective amount of the compound, composition, nucleic acid, vector, or cell of any examples herein, particularly examples 1-53.

Example 88: A method of killing bacteria, the method comprising exposing the bacteria to an effective amount of the compound, composition, nucleic acid, vector, or cell of any examples herein, particularly examples 1-53.

Example 89: A method for treating, preventing, inhibiting, and/or ameliorating a disease or disorder in a plant or a subject in need thereof, the method comprising administering to the subject a therapeutically effective amount of the compound, composition, nucleic acid, vector, or cell of any examples herein, particularly examples 1-53.

Example 90: The method of any examples herein, particularly example 89, wherein the disease or disorder comprises an infection, such as a microbial infection.

Example 91: A method for treating, preventing, inhibiting, and/or ameliorating a microbial infection in a plant or a subject, comprising administering to the plant or subject an effective amount of the compound, composition, nucleic acid, vector, or cell of any examples herein, particularly examples 1-53.

Example 82: The method of any examples herein, particularly examples 89-91, wherein the plant is a crop.

Example 93: The method of any examples herein, particularly examples 89-91, wherein the subject is an animal.

Example 94: The method of any examples herein, particularly example 93, wherein the animal is a companion animal, livestock, research animal, insect, or human.

Example 95: The method of any examples herein, particularly examples 85-94, wherein the compound, composition, nucleic acid, or vector is delivered via cultured Streptomyces.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The compositions and methods of the appended claims are not limited in scope by the specific compositions methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A compound defined by Formula I: 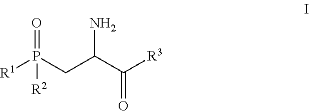
wherein
R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl, substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl, substituted or unsubstituted C₄-C₂₁ alkylaryl, NR^{x}R^{y}, or OR^{b};
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more canonical or non-canonical amino acids;
R^{a} and R^{b} are each independently hydrogen, substituted or unsubstituted C₁-C₁₀ alkyl, substituted or unsubstituted C₃-C₁₀ aryl, or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof;
with the proviso that the compound is not phosphonoalamide A, B, C, D, E, or F.

2. The compound of claim 1, wherein R¹ is OR^{a} and/or R² is OR^{b}.

3. The compound of claim 1, wherein the compound is defined by Formula II: 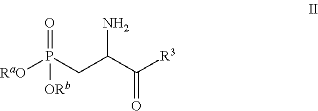
wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more canonical or non-canonical amino acids;
R^{a} and R^{b} are each independently hydrogen, substituted or unsubstituted C₁-C₁₀ alkyl, substituted or unsubstituted C₃-C₁₀ aryl, or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof;
with the proviso that the compound is not phosphonoalamide A, B, C, D, E, or F.

4. The compound of claim 1, wherein R^{a} and/or R^{b} is hydrogen.

5. The compound of claim 1, wherein the compound is defined by Formula III: 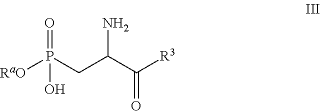
wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more canonical or non-canonical amino acids;
R^{a} is hydrogen, substituted or unsubstituted C₁-C₁₀ alkyl, substituted or unsubstituted C₃-C₁₀ aryl, or substituted or unsubstituted C₄-C₁₁ alkylaryl; and
R^{x} and R^{y} are independently selected from hydrogen, or substituted or unsubstituted C₁-C₅ alkyl, or substituted or unsubstituted C₁-C₅ acyl;
or a derivative or salt thereof;
with the proviso that the compound is not phosphonoalamide A, B, C, D, E, or F.

6. The compound of claim 1, wherein the compound is defined by Formula IV: 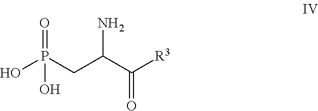
wherein
R³ is hydrogen, hydroxyl, halide, substituted or unsubstituted C₁-C₂₀ alkyl, substituted or unsubstituted C₁-C₂₀ acyl, substituted or unsubstituted C₁-C₂₀ alkoxy, substituted or unsubstituted C₁-C₂₀ amide, NR^{x}R^{y}, or one or more canonical or non-canonical amino acids; and
R^{x} and R^{y} are independently selected from hydrogen, or substituted or unsubstituted C₁-C₅ alkyl, or substituted or unsubstituted C₁-C₅ acyl;
or a derivative or salt thereof;
with the proviso that the compound is not phosphonoalamide A, B, C, D, E, or F.

7. The compound of claim 1, wherein R³ is one or more canonical or non-canonical amino acids.

8. The compound of claim 1, wherein R³ is one or more canonical or non-canonical amino acids, each canonical or non-canonical amino acid independently being selected from the group consisting of Alanine (Ala), Arginine (Arg), Asparagine (Asn), Cysteine (Cys), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), allothreonine (allo-Thr), Tryptophan (Trp), Tyrosine (Tyr), and Valine (Val).

9. The compound of claim 1, wherein the compound comprises PnAla-Val-Val, PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof.

10. The compound of claim 1, wherein the compound comprises PnAla-Ala-Val, PnAla-Val-Val, PnAla-Ala-Ile, PnAla-Thr-Val, a derivative or salt thereof, or a combination thereof.

11. The compound of claim 1, wherein the compound comprises PnAla-Ala-Val, PnAla-Ala-Thr, PnAla-Ala-Ser, PnAla-Ala-Leu, PnAla-Ala-Ile, PnAla-Ala-Met, PnAla-Ala-Arg, PnAla-Ala-Lys, PnAla-Ala-Gln, PnAla-Ala-His, PnAla-Ala-Ala, PnAla-Ala-Phe, PnAla-Ala-Trp, PnAla-Ala-Tyr, PnAla-Gly-Val, PnAla-Gly-Leu, PnAla-Gly-Ile, PnAla-Val-Ile, PnAla-Ser-Val, PnAla-Ser-Leu, PnAla-Ser-Ile, PnAla-Ser-Met, PnAla-Thr-Val, a derivative or salt thereof, or a combination thereof.

12. The compound of claim 1, wherein the compound comprises PnAla-Ala-Ala, PnAla-Ala-Val, PnAla-Ala-Ile, PnAla-Ala-Leu, PnAla-Ala-Met, PnAla-Ala-Phe, PnAla-Ala-Tyr, PnAla-Ala-Trp, PnAla-Ala-Ser, PnAla-Ala-Thr, PnAla-Ala-aThr, PnAla-Ala-Asn, PnAla-Ala-Gln, PnAla-Ala-Arg, PnAla-Ala-His, PnAla-Ala-Lys, PnAla-Ala-Glu, PnAla-Ala-Gly, PnAla-Ala-Pro, PnAla-Val-Val, PnAla-Val-Ile, PnAla-Val-Leu, PnAla-Val-Met, PnAla-Val-Phe, PnAla-Val-Tyr, PnAla-Val-Trp, PnAla-Val-aThr, PnAla-Val-Gln, PnAla-Val-Pro, PnAla-Ile-Ile, PnAla-Ile-Phe, PnAla-Ile-aThr, PnAla-Leu-Leu, PnAla-Met-Val, PnAla-Met-Leu, PnAla-Met-Met, PnAla-Met-Trp, PnAla-Phe-Val, PnAla-Phe-Ile, PnAla-Phe-Leu, PnAla-Phe-Met, PnAla-Phe-Phe, PnAla-Phe-Tyr, PnAla-Phe-Trp, PnAla-Phe-Thr, PnAla-Phe-aThr, PnAla-Tyr-Val, PnAla-Tyr-Leu, PnAla-Tyr-Met, PnAla-Tyr-Phe, PnAla-Tyr-Tyr, PnAla-Tyr-Trp, PnAla-Tyr-Gly, PnAla-Trp-Val, PnAla-Trp-Ile, PnAla-Trp-Leu, PnAla-Trp-Met, PnAla-Trp-Phe, PnAla-Trp-Tyr, PnAla-Trp-Trp, PnAla-Trp-Thr, PnAla-Trp-Lys, PnAla-Trp-Gly, PnAla-Ser-Val, PnAla-Ser-Ile, PnAla-Ser-Leu, PnAla-Ser-Met, PnAla-Ser-Phe, PnAla-Ser-Tyr, PnAla-Ser-Ser, PnAla-Ser-Thr, PnAla-Ser-aThr, PnAla-Thr-Val, PnAla-Thr-Ile, PnAla-Thr-Leu, PnAla-Thr-Phe, PnAla-Thr-Trp, PnAla-aThr-Val, PnAla-aThr-Ile, PnAla-aThr-Leu, PnAla-aThr-Met, PnAla-aThr-Phe, PnAla-aThr-aThr, PnAla-Gln-Val, PnAla-Gln-Ile, PnAla-Gln-Leu, PnAla-His-Phe, PnAla-His-Trp, PnAla-Gly-Val, PnAla-Gly-Ile, PnAla-Gly-Leu, PnAla-Gly-Met, PnAla-Gly-Phe, PnAla-Gly-Tyr, PnAla-Gly-Trp, PnAla-Gly-aThr, PnAla-Gly-Gly, a derivative or salt thereof, or a combination thereof.

13. The compound of claim 1, wherein the compound is a salt.

14.-18. (canceled)

19. The compound of claim 1, wherein the compound is a potassium salt, sodium salt, calcium salt, iron salt, ammonium salt, or a combination thereof.

20. The compound of claim 1, wherein the compound comprises an agriculturally acceptable salt thereof and/or a pharmaceutically acceptable salt thereof.

21. A compound comprising a head group and a tail group, the head group being bound to the tail group using one or more enzymes derived from Streptomyces, wherein the head group comprises a phosphonic acid, a phosphinic acid, or a derivative thereof, and the tail group comprises one or more (canonical or non-canonical) amino acids, with the proviso that the compound is not phosphonoalamide A, B, C, D, E, or F.

22.-34. (canceled)

35. A composition comprising the compound of claim 1, wherein the composition exhibits antimicrobial activity and/or wherein the composition is formulated for delivery to a plant or animal.

36.-51. (canceled)

52. A cell comprising the compound of claim 1, wherein the cell comprises a Streptomyces cell.

53.-56. (canceled)

57. A method of making a compound comprising a head group and a tail group, the head group being bound to the tail group, wherein the head group comprises a phosphonic acid, a phosphinic acid, or a derivative thereof, and the tail group comprises one or more (canonical or non-canonical) amino acids, using one or more enzymes derived from Streptomyces, with the proviso that the compound is not phosphonoalamide A, B, C, D, E, or F.

58.-77. (canceled)

78. A method of use of the compound of claim 1, wherein the method comprises using the compound, composition, nucleic acid, vector, or cell as an antimicrobial, herbicide, pesticide, or combination thereof to control an undesirable population.

79.-95. (canceled)


SEQUENCE LISTING (21 sequences)

SEQ ID NO: 1 - DNA, 21 bp, synthetic construct
  source 1..21: mol_type = other DNA
  misc_feature 1..21: note = Description of sequence: Primer, S515-pepM-screen-F
gatgaacagcttcgtcgccac

SEQ ID NO: 2 - DNA, 19 bp, synthetic construct
  source 1..19: mol_type = other DNA
  misc_feature 1..19: note = Description of sequence: Primer, S515-pepM-screen-R
ccctggttggcgtagatga

SEQ ID NO: 3 - DNA, 65 bp, synthetic construct
  source 1..65: mol_type = other DNA
  misc_feature 1..65: note = Description of sequence: Primer, orf1-left-Redswap-F
tccgccaccgcacggggcggcgcgggcggtggggcctcacctaggtcgaggttgacatcttttgc

SEQ ID NO: 4 - DNA, 67 bp, synthetic construct
  source 1..67: mol_type = other DNA
  misc_feature 1..67: note = Description of sequence: Primer, orf1-left-Redswap-R
tacattatacgaagttatcaagaagcttgagctcggatccctaggtcagaagaactcgtcaagaagg

SEQ ID NO: 5 - DNA, 67 bp, synthetic construct
  source 1..67: mol_type = other DNA
  misc_feature 1..67: note = Description of sequence: Primer, pnaA-left-Redswap-R
tggtgagggtggtgaggacggaacgggtacggcgggccgcctaggtcagaagaactcgtcaagaagg

SEQ ID NO: 6 - DNA, 26 bp, synthetic construct
  source 1..26: mol_type = other DNA
  misc_feature 1..26: note = Description of sequence: Primer, pET28b-NdeI-R
catatggctgccgcgcggcaccaggc

SEQ ID NO: 7 - DNA, 26 bp, synthetic construct
  source 1..26: mol_type = other DNA
  misc_feature 1..26: note = Description of sequence: Primer, pET28b-XhoI-F
ctcgagcaccaccaccaccaccactg

SEQ ID NO: 8 - DNA, 21 bp, synthetic construct
  source 1..21: mol_type = other DNA
  misc_feature 1..21: note = Description of sequence: Primer, 2ST-F
tgcattggattggaagtacag

SEQ ID NO: 9 - DNA, 21 bp, synthetic construct
  source 1..21: mol_type = other DNA
  misc_feature 1..21: note = Description of sequence: Primer, 2ST-R
attggaagtggataacggatc

SEQ ID NO: 10 - DNA, 41 bp, synthetic construct
  source 1..41: mol_type = other DNA
  misc_feature 1..41: note = Description of sequence: Primer, pET28b-NHis-PnaB-F
tgccgcgcggcagccatatgtcaggtgacctccggccgctc

SEQ ID NO: 11 - DNA, 44 bp, synthetic construct
  source 1..44: mol_type = other DNA
  misc_feature 1..44: note = Description of sequence: Primer, pET28b-NHis-PnaB-R
tggtggtggtggtgctcgagtcatcccgtcgccccgaagtcctc

SEQ ID NO: 12 - DNA, 41 bp, synthetic construct
  source 1..41: mol_type = other DNA
  misc_feature 1..41: note = Description of sequence: Primer, pET28b-NHis-PnaC-F
tgccgcgcggcagccatatgacccgtcacatcgccttcctg

SEQ ID NO: 13 - DNA, 44 bp, synthetic construct
  source 1..44: mol_type = other DNA
  misc_feature 1..44: note = Description of sequence: Primer, pET28b-NHis-PnaC-R
tggtggtggtggtgctcgagtcagtgcgagtcggcggtccggac

SEQ ID NO: 14 - DNA, 36 bp, synthetic construct
  source 1..36: mol_type = other DNA
  misc_feature 1..36: note = Description of sequence: Primer, 2ST-PnaA-F
ttatccacttccaattcacctgacatcgtccatcac

SEQ ID NO: 15 - DNA, 36 bp, synthetic construct
  source 1..36: mol_type = other DNA
  misc_feature 1..36: note = Description of sequence: Primer, 2ST-PnaA-R
ttccaatccaatgcaatgcccgaccagcaggtcgac

SEQ ID NO: 16 - DNA, 44 bp, synthetic construct
  source 1..44: mol_type = other DNA
  misc_feature 1..44: note = Description of sequence: Primer, S515-pepM-F
gcctggtgccgcgcggcagccatatgttcaagcccgtttcgtac

SEQ ID NO: 17 - DNA, 46 bp, synthetic construct
  source 1..46: mol_type = other DNA
  misc_feature 1..46: note = Description of sequence: Primer, S515-pepM-R
agtggtggtggtggtggtgctcgagtcacgcggactcgagctcctg

SEQ ID NO: 18 - DNA, 40 bp, synthetic construct
  source 1..40: mol_type = other DNA
  misc_feature 1..40: note = Description of sequence: Primer, pET28B-NHis-SCO4927-Fd
tgccgcgcggcagccatatgactcgcactcccgtgaacgt

SEQ ID NO: 19 - DNA, 40 bp, synthetic construct
  source 1..40: mol_type = other DNA
  misc_feature 1..40: note = Description of sequence: Primer, pET28B-NHis-SCO4927-Rv
tggtggtggtggtgctcgagtcagatgaggccgagaccgc

SEQ ID NO: 20 - DNA, 38 bp, synthetic construct
  source 1..38: mol_type = other DNA
  misc_feature 1..38: note = Description of sequence: Primer, pET28B-NHis-SerA-F
tgccgcgcggcagccatatggcaaaggtatcgctggag

SEQ ID NO: 21 - DNA, 41 bp, synthetic construct
  source 1..41: mol_type = other DNA
  misc_feature 1..41: note = Description of sequence: Primer, pET28B-NHis-SerA-R
tggtggtggtggtgctcgagttagtacagcagacgggcgcg